(19) United States
(12) Patent Application Publication
(10) Pub. No.: US 2010/0204277 A1
(43) Pub. Date: Aug. 12, 2010

(54) Compositions and Methods for Controlling Infestation

(75) Inventors: Vernon Morrison Bowles, Glen Iris (AU); Paul MacLeman, Parkville (AU)

Correspondence Address: MCANDREWS HELD & MALLOY, LTD, 500 WEST MADISON STREET, SUITE 3400, CHICAGO, IL, 60661, US

(73) Assignee: HATCHTECH PTY LTD., Parkville (AU)

(21) Appl. No.: 12/594,688

(22) PCT Filed: Apr. 5, 2007
(86) PCT No.: PCT/IB07/03226

§ 371 Date: Feb. 8, 2010

(51) Int. Cl. A01N 43/40; G01N 33/00

(52) U.S. Cl. 514/332; 436/96

(57) ABSTRACT

The present invention is directed to methods of treating pest infestation by inhibiting metabolic processes of the pest such as for example, processes involved in invertebrate remodelling.

### FIELD OF THE INVENTION

The present invention relates to compositions and methods for inhibiting the activity of an enzyme or enzymes that are directly or indirectly involved in invertebrate remodelling events. In particular, the invention relates to compositions and methods for controlling invertebrate multicellular organisms having cross-linked protein structures that include but are not limited to eggs, sheaths, carapaces, exoskeletons, cysts, cocoons or ootheca. The invention also provides methods of inhibiting processes such as apolysis, ecdysis, egg hatching, excystment, exsheathment and metamorphosis. The invention also provides methods and compositions for preventing, treating or controlling infestations of an invertebrate pest that undergoes remodelling events.

### BACKGROUND OF THE INVENTION

Pests that undergo remodelling events such as egg hatching, moulting and/or metamorphosis from pupae to adult, cause significant problems in a wide variety of situations. For example, pests that undergo such remodelling events may externally infest humans or animals and annoy, bite and/or cause infections, particularly of humans and domesticated animals. These pests may also internally infest humans and animals causing infection, gastrointestinal problems, swelling, and/or lymphatic problems and blood loss. Pests that undergo remodelling events may also infest plants and their larvae or other life cycle stages can eat leaves, flowers, roots and fruit causing significant damage to commercially important crops. Other pests that undergo these remodelling events infest the environment and cause illness to humans or animals or property damage. For example, termites cause significant property damage and the presence of dust or house mites can cause asthma in humans.

A large number of pesticides are known for controlling or eliminating plant, human, animal and environmental pests. These pesticides may be used in the form of aerosols, space sprays, liquids, soaps, shampoos, wettable powders, granules, baits, dusts, tablets and the like.

Conventional control methods for pests rely on the use of chemical pesticides such as chlorinated hydrocarbons (DDT, endosulfan, etc.), synthetic and natural pyrethrins (pyrethrin, permethrin, cypermethrin, deltamethrin), insect growth regulators that are known to interfere with chitin synthesis, insecticidal bacterial toxins (Bacillus thuringiensis (Bt) toxins) and nematicides including both fumigant and non-fumigant (ie formulated granules or liquids). However, significant problems are associated with the use of pesticides including commonality in target organs and modes of action leading to the development of resistance by the target pest, the need for increased pesticide use, the persistence of chemicals in the environment and in plant and animal tissues, harmful effects on host and non-target organisms and lack of ovicidal activity.

The modern approach to the control of pest species relies on a combination of factors including the use of appropriate management strategies that aim to minimise the use of pesticides but still afford effective control. This change in the approach to control has been necessary due to the overuse and over reliance on chemicals leading to major problems of resistance to many of the commonly used chemistries. Furthermore due to their often quite specific modes of action, a number of the chemicals used in the field often only target specific stages of the lifecycle when the appropriate target is being expressed. For example a pesticide may control pests by killing larvae only after they emerge from eggs, or killing the pest during its pupal or adult life stages. However, any eggs present at application of the pesticide are often unaffected and upon maturing and hatching result in re-infestation of the plant, human, animal or environment. This results in repeated application of pesticide or prolonged exposure to the pesticide being required for continued control of the pest. This is not only inconvenient and costly but also increases the risks to the environment, plant, human or animal.

Accordingly, there remains a need for providing alternative methods and compositions that are effective in preventing or controlling remodelling events associated with development of pests throughout all of the different developmental stages to provide more efficient and effective control.

### SUMMARY OF THE INVENTION

The present invention is directed to methods of treating pest infestation by inhibiting metabolic processes of the pest such as for example, processes involved in invertebrate remodelling. In specific embodiments, the methods of the invention comprise decreasing exsheathment of an invertebrate by externally contacting a pest with a compound of formula (I):

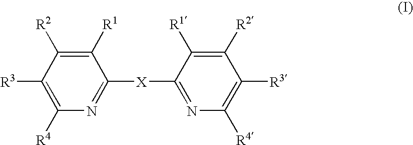

wherein X is selected from a covalent bond, —C(R⁵)₂—, —Z— or —C(R⁵)₂—Z—C(R⁵)₂—;

R¹ and R^{1′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R¹⁹ taken together are —C(R⁵)₂—, —C(R⁵)₂—C(R⁵)₂—, —CR⁵═CR⁵—, C(O), C(S) or NH;

R², R^{2′}, R³, R^{3′}, R⁴ and R^{4′} are independently selected from hydrogen, C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or

R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;

each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;

each R⁶ is independently selected from hydrogen and halogen; and

Z is selected from a covalent bond, —NH—, —O—, —S—, —C(O)— and —C(S)—;

a pharmaceutically, veterinary or agriculturally acceptable salt thereof in an amount effective to inhibit or decrease the rate of exsheathment of said invertebrate.

Other embodiments of the invention comprise methods of treating pest infestation comprising decreasing excystment of an invertebrate by externally contacting a pest with a compound of formula:

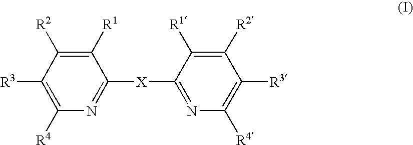

wherein X is selected from a covalent bond, —C(R⁵)₂—, —Z—or —C(R⁵)₂—Z—C(R⁵)₂—;

R¹ and R^{1′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R¹⁹ taken together are —C(R⁵)₂—, —C(R⁵)₂—C(R⁵)₂—, —CR⁵═CR⁵—, C(O), C(S) or NH;

R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₉alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or

R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;

each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;

each R⁶ is independently selected from hydrogen and halogen; and

Z is selected from a covalent bond, —NH—, —O—, —S—, —C(O)— and —C(S)—;

a pharmaceutically, veterinary or agriculturally acceptable salt thereof in an amount effective to inhibit or otherwise decrease the rate of excystment of said invertebrate.

The invention further contemplates methods of treating pest infestation comprising decreasing apolysis of an invertebrate by externally contacting a pest with a compound of formula:

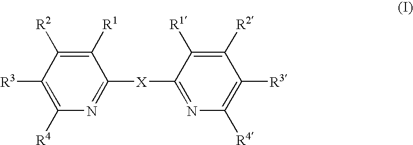

wherein X is selected from a covalent bond, —C(R⁵)₂—, —Z— or —C(R⁵)₂—Z—C(R⁵)₂—;

R¹ and R^{1′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R¹⁹ taken together are —C(R⁵)₂—, —C(R⁵)₂—C(R⁵)₂—, —CR⁵═CR⁵—, C(O), C(S) or NH;

R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or

R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;

each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;

each R⁶ is independently selected from hydrogen and halogen; and

Z is selected from a covalent bond, —NH—, —O—, —S—, —C(O)— and —C(S)—;

a pharmaceutically, veterinary or agriculturally acceptable salt thereof in an amount effective to inhibit or otherwise decrease the rate of apolysis of said invertebrate.

In still further embodiments, the methods of the invention involve treating pest infestation comprising inhibiting metamorphosis of an invertebrate by externally contacting said pest with a compound of formula:

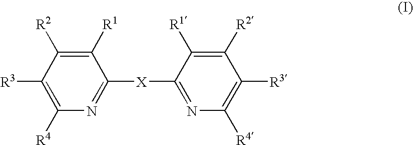

wherein X is selected from a covalent bond, —C(R⁵)₂—, —Z— or —C(R⁵)₂—Z—C(R⁵)₂—;

R¹ and R^{1′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R¹⁹ taken together are —C(R⁵)₂—, —C(R⁵)₂—C(R⁵)₂—, —CR⁵═CR⁵—, C(O), C(S) or NH;

R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or

R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;

each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;

each R⁶ is independently selected from hydrogen and halogen; and

Z is selected from a covalent bond, —NH—, —O—, —S—, —C(O)— and —C(S)—;

or a pharmaceutically, veterinary or agriculturally acceptable salt thereof in an amount effective to inhibit or otherwise decrease the rate of metamorphosis of said invertebrate.

In the methods of the invention, it is preferred that the compound is a metal chelating agent, wherein the metal chelating agent has at least two polar atoms capable of simultaneously coordinating with a metal ion, has a clogP value of /1 and ≦4; and/or and a molar refractivity in the range of 40 to 90 cm³/mole. In specific embodiments, the metal chelating agent is not 1,10-phenanthroline. In other embodiments, the metal chelating agent is not a dipyridyl compound.

The methods of the invention contemplate the use of multiple pesticides and interventions for treating infestations. In specific embodiments, the methods further comprises contacting said pest with a second pesticide.

Other aspects of the invention comprise methods of treating pest infestation comprising decreasing exsheathment of an invertebrate by externally contacting a pest with a compound of formula (II):

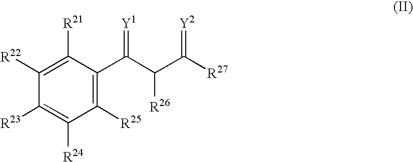

wherein Y¹ and Y² are independently selected form O, NR₂₈, or S;
R²¹, R²², R²³ and R²⁴ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl, N(C₁₋₆alkyl)₂ or a carbocyclic or heterocyclic ring; or
R²¹ and R²² or R²² and R²³ and R²⁴ taken together with the carbon atoms to which they are attached form a 5 or 6 membered carbocyclic or heterocyclic ring;
R²⁵ and R²⁶ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl or N(C₁₋₆alkyl)₂; or
R²⁵ and R²⁶ together with the carbon atoms to which they are attached form a 5 or 6 membered carbocyclic or heterocyclic ring;

R²⁷ is C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, C(R²⁹)₂, N(R³⁰)₂, or a 5 or 6 membered carbocyclic ring or heterocyclic ring;
R²⁸ is hydrogen, C₁₋₆alkyl, a branched-chain C₁₋₆alkyl;
R²⁹ is hydrogen or halogen; and
each R³⁰ is independently selected from hydrogen, C₁₋₆alkyl, a branched-chain C₁₋₆alkyl, C₂₋₆alkenyl, C₂₋₆alkynyl, a 5 or 6 membered carbocyclic ring or heterocyclic ring;

or a pharmaceutically, veterinary or agriculturally acceptable salt thereof in an amount effective to inhibit or decrease the rate of exsheathment of said invertebrate.

Still another embodiment of the invention describes a method of treating pest infestation comprising decreasing excystment of an invertebrate by externally contacting a pest with a compound of formula (II):

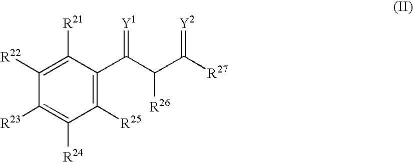

wherein Y¹ and Y² are independently selected form O, NR₂₈, or S;
R²¹, R²², R²³ and R²⁴ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl, N(C₁₋₆alkyl)₂ or a carbocyclic or heterocyclic ring; or
R²¹ and R²² or R²² and R²³ and R²⁴ taken together with the carbon atoms to which they are attached form a 5 or 6 membered carbocyclic or heterocyclic ring;
R²⁵ and R²⁶ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl or N(C₁₋₆alkyl)₂; or
R²⁵ and R²⁶ together with the carbon atoms to which they are attached form a 5 or 6 membered carbocyclic or heterocyclic ring;
R²⁷ is C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, C(R²⁹)₂, N(R³⁰)₂, or a 5 or 6 membered carbocyclic ring or heterocyclic ring;
R²⁸ is hydrogen, a C₁₋₆alkyl, or a branched-chain C₁₋₆alkyl;
R²⁹ is hydrogen or halogen; and
each R³⁰ is independently selected from hydrogen, C₁₋₆alkyl, a branched-chain C₁₋₆alkyl, C₂₋₆alkenyl, C₂₋₆alkynyl, a 5 or 6 membered carbocyclic ring or heterocyclic ring;

or a pharmaceutically, veterinary or agriculturally acceptable salt thereof in an amount effective to inhibit or otherwise decrease the rate of excystment of said invertebrate.

The invention also contemplates methods of treating pest infestation comprising decreasing apolysis of an invertebrate by externally contacting a pest with a compound of formula (II):

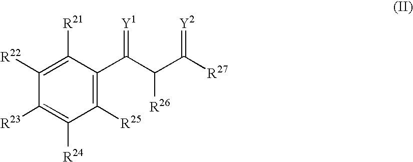

wherein Y¹ and Y² are independently selected form O, NR₂₈, or S;
R²¹, R²², R²³ and R²⁴ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl, N(C₁₋₆alkyl)₂ or a carbocyclic or heterocyclic ring; or
R²¹ and R²² or R²² and R²³ and R²⁴ taken together with the carbon atoms to which they are attached form a 5 or 6 membered carbocyclic or heterocyclic ring;
R²⁵ and R²⁶ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl or N(C₁₋₆alkyl)₂; or
R²⁵ and R²⁶ together with the carbon atoms to which they are attached form a 5 or 6 membered carbocyclic or heterocyclic ring;
R²⁷ is C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, C(R²⁹)₂, N(R³⁰)₂, or a 5 or 6 membered carbocyclic ring or heterocyclic ring;
R²⁸ is hydrogen, a C₁₋₆alkyl, or a branched-chain C₁₋₆alkyl;
R²⁹ is hydrogen or halogen; and
each R³⁰ is independently selected from hydrogen, C₁₋₆alkyl, a branched-chain C₁₋₆alkyl, C₂₋₆alkenyl, C₂₋₆alkynyl, a 5 or 6 membered carbocyclic ring or heterocyclic ring;

or a pharmaceutically, veterinary or agriculturally acceptable salt thereof in an amount effective to inhibit or decrease the rate of apolysis of said invertebrate.

Yet another alternative method of treating pest infestation comprises inhibiting metamorphosis of an invertebrate by externally contacting said pest with a compound of formula:

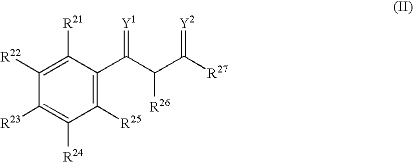

wherein Y¹ and Y² are independently selected form O, NR₂₈, or S;
R²¹, R²², R²³ and R²⁴ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl, N(C₁₋₆alkyl)₂ or a carbocyclic or heterocyclic ring; or
R²¹ and R²² or R²² and R²³ and R²⁴ taken together with the carbon atoms to which they are attached form a 5 or 6 membered carbocyclic or heterocyclic ring;
R²⁵ and R²⁶ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl or N(C₁₋₆alkyl)₂; or
R²⁵ and R²⁶ together with the carbon atoms to which they are attached form a 5 or 6 membered carbocyclic or heterocyclic ring;
R²⁷ is C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, C(R²⁹)₂, N(R³⁰)₂, or a 5 or 6 membered carbocyclic ring or heterocyclic ring;
R²⁸ is hydrogen, a C₁₋₆alkyl, or a branched-chain C₁₋₆alkyl;
R²⁹ is hydrogen or halogen; and
each R³⁰ is independently selected from hydrogen, C₁₋₆alkyl, a branched-chain C₁₋₆alkyl, C₂₋₆alkenyl, C₂₋₆alkynyl, a 5 or 6 membered carbocyclic ring or heterocyclic ring;

or a pharmaceutically, veterinary or agriculturally acceptable salt thereof in an amount effective to inhibit or otherwise decrease the rate of metamorphosis of said invertebrate.

Again, in each of the foregoing methods, the compound is preferably a metal chelating agent, wherein the metal chelating agent has at least two polar atoms capable of simultaneously coordinating with a metal ion, has a clogP value of /1 and ≦4; and/or and a molar refractivity in the range of 40 to 90 cm³/mole. However, it is contemplated that the metal chelating agent is not 1,10-phenanthroline. The foregoing methods may further comprise contacting the pest with a second, third, fourth or more pesticides. Further, the pest may be treated multiple times with the various pesticides described herein.

In preferred embodiments of the invention, the methods described herein produce a greater decrease in the rate of exsheathment, excystment, apolysis or metamorphosis than is observed with the administration of 1,10 phenanthroline.

In particularly preferred embodiments of the invention, the methods are employed for killing an invertebrate pest, said method comprising externally contacting said pest with a compound of formula (I):

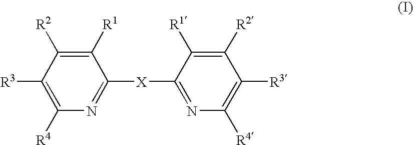

wherein X is selected from a covalent bond, —C(R⁵)₂—, —Z— or —C(R⁵)₂—Z—C(R⁵)₂—;

R¹ and R^{1′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R¹⁹ taken together are —C(R⁵)₂—, —C(R⁵)₂—C(R⁵)₂—, —CR⁵═CR⁵—, C(O), C(S) or NH;

R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or

R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;

each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;

each R⁶ is independently selected from hydrogen and halogen; and

Z is selected from a covalent bond, —NH—, —O—, —S—, —C(O)— and —C(S)—;

or a pharmaceutically, veterinary or agriculturally acceptable salt thereof in an amount effective to kill said invertebrate.

Other preferred embodiments are directed to methods of killing an invertebrate pest, said method comprising externally contacting said pest with a compound of formula (II):

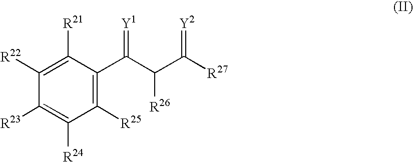

wherein Y¹ and Y² are independently selected form O, NR₂₈, or S;
R²¹, R²², R²³ and R²⁴ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl, N(C₁₋₆alkyl)₂ or a carbocyclic or heterocyclic ring; or
R²¹ and R²² or R²² and R²³ and R²⁴ taken together with the carbon atoms to which they are attached form a 5 or 6 membered carbocyclic or heterocyclic ring;
R²⁵ and R²⁶ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl or N(C₁₋₆alkyl)₂; or
R²⁵ and R²⁶ together with the carbon atoms to which they are attached form a 5 or 6 membered carbocyclic or heterocyclic ring;
R²⁷ is C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, C(R²⁹)₂, N(R³⁰)₂, or a 5 or 6 membered carbocyclic ring or heterocyclic ring;
R²⁸ is hydrogen, a C₁₋₆alkyl, or a branched-chain C₁₋₆alkyl;
R²⁹ is hydrogen or halogen; and
each R³⁰ is independently selected from hydrogen, C₁₋₆alkyl, a branched-chain C₁₋₆alkyl, C₂₋₆alkenyl, C₂₋₆alkynyl, a 5 or 6 membered carbocyclic ring or heterocyclic ring;

or a pharmaceutically, veterinary or agriculturally acceptable salt thereof in an amount effective to kill said invertebrate.

In the killing methods of the invention, the invertebrate pest is selected from the group consisting of nematodes, trematodes, cestodes, lice, fleas, mites and scabies, moths, beetles, caterpillars butterflies, termites, arachnids, cockroaches, centipedes, fleas and mites.

It is preferred that the methods are such that they are used to kill at least some of the invertebrate pests that are infesting a host. In preferred embodiments, the methods produce results in which at least 25% of the pests in a given infestation are killed. In other embodiments, at least 30%, of the pest population is killed. In still other embodiments, at least 50% of the invertebrate population in a given infestation is killed. In still other preferred embodiments, at least 75% of the invertebrate pest population in a given infestation is killed.

Also contemplated are methods of inhibiting a remodelling event in an invertebrate population comprising contacting said invertebrate population with a compound of formula (I):

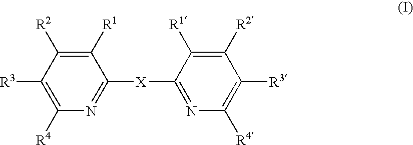

wherein X is selected from a covalent bond, —C(R⁵)₂—, —Z— or —C(R⁵)₂—Z—C(R⁵)₂—;

R¹ and R^{1′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R¹⁹ taken together are —C(R⁵)₂—, —C(R⁵)₂—C(R⁵)₂—, —CR⁵═CR⁵—, C(O), C(S) or NH;

R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or

R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;

each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;

each R⁶ is independently selected from hydrogen and halogen; and

Z is selected from a covalent bond, —NH—, —O—, —S—, —C(O)— and —C(S)—;

or a pharmaceutically, veterinary or agriculturally acceptable salt thereof in an amount effective to inhibit said invertebrate remodelling event, wherein said invertebrate remodelling event is not egg hatching and said invertebrate is not an ectoparasitic insect.

Another method of the invention is for inhibiting a remodelling event in an invertebrate population comprising internally contacting said invertebrate population with a compound of formula (II):

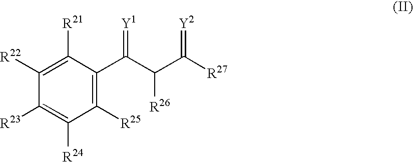

wherein Y¹ and Y² are independently selected form O, NR₂₈, or S;
R²¹, R²², R²³ and R²⁴ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl, N(C₁₋₆alkyl)₂ or a carbocyclic or heterocyclic ring; or
R²¹ and R²² or R²² and R²³ and R²⁴ taken together with the carbon atoms to which they are attached form a 5 or 6 membered carbocyclic or heterocyclic ring;
R²⁵ and R²⁶ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl or N(C₁₋₆alkyl)₂; or
R²⁵ and R²⁶ together with the carbon atoms to which they are attached form a 5 or 6 membered carbocyclic or heterocyclic ring;
R²⁷ is C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, C(R²⁹)₂, N(R³⁰)₂, or a 5 or 6 membered carbocyclic ring or heterocyclic ring;
R²⁸ is hydrogen, C₁₋₆alkyl, or a branched-chain C₁₋₆alkyl;
R²⁹ is hydrogen or halogen; and
each R³⁰ is independently selected from hydrogen, C₁₋₆alkyl, a branched-chain C₁₋₆alkyl, C₂₋₆alkenyl, C₂₋₆alkynyl, a 5 or 6 membered carbocyclic ring or heterocyclic ring;

or a pharmaceutically, veterinary or agriculturally acceptable salt thereof in an amount effective to inhibit said invertebrate remodelling event, wherein said invertebrate remodelling event is not egg hatching and said invertebrate is not an ectoparasitic insect.

The invention also provides methods of inhibiting egg hatching in a non-ectoparasitic invertebrate an invertebrate population comprising contacting said invertebrate with a compound of formula (I):

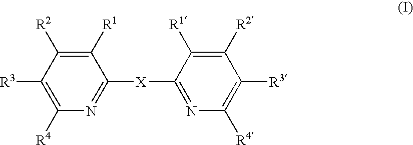

wherein X is selected from a covalent bond, —C(R⁵)₂—, —Z— or —C(R⁵)₂—Z—C(R⁵)₂—;

R¹ and R^{1′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R¹⁹ taken together are —C(R⁵)₂—, —C(R⁵)₂—C(R⁵)₂—, —CR⁵═CR⁵—, C(O), C(S) or NH;

R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or

R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;

each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl,

SO₃H, SO₃C₁₋₆alkyl, NH₂, NHC₁₋₆alkyl or N(C₁₋₆alkyl)₂;

each R⁶ is independently selected from hydrogen and halogen; and

Z is selected from a covalent bond, —NH—, —O—, —S—, —C(O)— and —C(S)—;

or a pharmaceutically, veterinary or agriculturally acceptable salt thereof in an amount effective to inhibit said egg hatching.

Also provided is a method of inhibiting egg hatching in a non-ectoparasitic invertebrate an invertebrate population comprising contacting said invertebrate with a compound of formula (II):

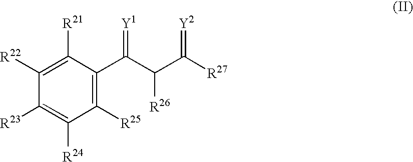

wherein Y¹ and Y² are independently selected form O, NR₂₈, or S;
R²¹, R²², R²³ and R²⁴ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl, N(C₁₋₆alkyl)₂ or a carbocyclic or heterocyclic ring; or
R²¹ and R²² or R²² and R²³ and R²⁴ taken together with the carbon atoms to which they are attached form a 5 or 6 membered carbocyclic or heterocyclic ring;
R²⁵ and R²⁶ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl or N(C₁₋₆alkyl)₂; or
R²⁵ and R²⁶ together with the carbon atoms to which they are attached form a 5 or 6 membered carbocyclic or heterocyclic ring;
R²⁷ is C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, C(R²⁹)₂, N(R³⁰)₂, or a 5 or 6 membered carbocyclic ring or heterocyclic ring;
R²⁸ is hydrogen, C₁₋₆alkyl, or a branched-chain C₁₋₆alkyl;
R²⁹ is hydrogen or halogen; and
each R³⁰ is independently selected from hydrogen, C₁₋₆alkyl, a branched-chain C₁₋₆alkyl, C₂₋₆alkenyl, C₂₋₆alkynyl, a 5 or 6 membered carbocyclic ring or heterocyclic ring;

or a pharmaceutically, veterinary or agriculturally acceptable salt thereof in an amount effective to inhibit said egg hatching.

Preferably, the non-ectoparasitic invertebrate is selected from the group consisting of nematodes, trematodes and cestodes. In more preferred embodiments, the invertebrate is a nematode. Preferably, the nematode is in inhibited in its larval stage.

The present invention provides additional methods for identifying and selecting a chelating agent as a candidate inhibitor of invertebrate remodelling events from a collection of metal chelating agents;

said method comprising selecting a metal chelating agent that has at least two polar atoms capable of simultaneously coordinating with a metal ion and
i) a clogP value of /1 and ≦4; and/or
ii) a molar refractivity in the range of 40 to 90 cm³/mole.

In this manner, the methods of the invention may be used for screening combinatorial libraries for rational drug design of agents that can be used as inhibitors of invertebrate remodelling and/or as pesticides in general.

Yet another aspect of the invention involves screening assays in which agents are identified and/or selected. Such methods involve identification or selection of a chelating agent as a candidate inhibitor of invertebrate remodelling events from a collection of metal chelating agents of formula (II):

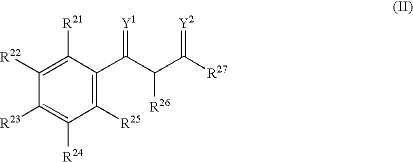

wherein Y¹ and Y² are independently selected form O, NR²⁸, or S;

R²¹, R²², R²³ and R²⁴ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R²⁹)₃, CO₂H, CO₂C₁₋₆alkyl, SO₃H, SO₃C₁₋₆alkyl, NH₂, NHC₁₋₆alkyl, _{N(C1-6alkyl)2} or a carbocyclic or heterocyclic ring; or

R₂₁ and R₂₂ or R₂₂ and R₂₃ and R₂₄ taken together with the carbon atoms to which they are attached form a 5 or 6 membered carbocyclic or heterocyclic ring;

R₂₅ and R₂₆ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R²⁹)₃, CO₂H, CO₂C₁₋₆alkyl, SO₃H, SO₃C₁₋₆alkyl NH₂, NHC₁₋₆₋alkyl or N(C₁₋₆alkyl)₂; or

R²⁵ and R²⁶ together with the carbon atoms to which they are attached form a 5 or 6 membered carbocyclic or heterocyclic ring;

R₂₇ is C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, C(R₂₉)₂, N(R₃₀)₂, or a 5 or 6 membered carbocyclic ring or heterocyclic ring;

R₂₈ is hydrogen, C₁₋₆alkyl, or a branched-chain C₁₋₆alkyl;

R₂₉ is hydrogen or halogen; and

each R₃₀ is independently selected from hydrogen, C₁₋₆alkyl, a branched-chain C₁₋₆alkyl, C₂₋₆alkenyl, C₂₋₆alkynyl, a 5 or 6 membered carbocyclic ring or heterocyclic ring;

or a pharmaceutically, veterinary or agriculturally acceptable salt thereof;

said method comprising selecting a metal chelating agent of formula (II) that has at least two polar atoms capable of simultaneously coordinating with a metal ion and
i) a clogP value of /1 and ≦4; and/or
ii) a molar refractivity in the range of 40 to 90 cm3/mole.

In still further embodiments, the methods of the invention involve screening methods of identifying or selecting a chelating agent as a candidate inhibitor of invertebrate remodelling events from a collection of metal chelating agents of formula I:

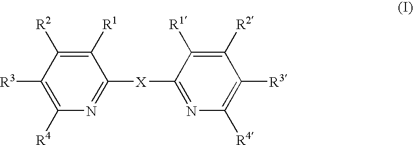

wherein X is selected from a covalent bond, —C(R⁵)₂—, —Z— or —C(R⁵)₂—Z—C(R⁵)₂—;

R¹ and R^{1′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R¹⁹ taken together are —C(R⁵)₂—, —C(R⁵)₂—C(R⁵)₂—, —CR⁵═CR⁵—, C(O), C(S) or NH;

R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or

R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;

each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;

each R⁶ is independently selected from hydrogen and halogen; and

Z is selected from a covalent bond, —NH—, —O—, —S—, —C(O)— and —C(S)—;

said method comprising selecting a metal chelating agent of formula (I) that has at least two polar atoms capable of simultaneously coordinating with a metal ion and
i) a clogP value of /1 and ≦4; and/or
ii) a molar refractivity in the range of 40 to 90 cm3/mole.

The screening assays of the invention may be combined with conventional biological assays employed to determine the efficacy of a given agent as a pesticide. As such, the screening assays above can be combined with assays designed to determine the effect on egg hatching, moulting, metamorphosis and the like as well as in vitro enzyme assays that determine the activity of one or more of the enzymes involved in one or more of the remodelling events. In specific embodiments, the screening assays may be combined with assays that determine the efficacy of the compounds as inhibitors of proteases and the like.

### BRIEF DESCRIPTION OF THE FIGURES

FIG. 1: shows a gelatine substrate SDS-PAGE analysis of protease activity of washings obtained from various samples of hair and lice eggs (egg shell washings ESW) following staining of the gel with Coomassie blue and destaining. Lane 1 shows protease activity detected in the washings obtained from unhatched lice eggs within 12 hours of hatching (sample 1) in the higher molecular weight region of the SDS gel, above 50 kDa (FIG. 1A, lane 1). A similar pattern of protease activity was detected in the washings taken from human hair samples following the removal of the louse eggs (sample 2) (FIG. 1A, lane 2). However, treatment of the hair with 1% sodium hypochlorite prior to collecting the washings (sample 3) completely removed the protease activity (FIG. 1A, lane 3). Hypochlorite treatment was also able to remove the extraneous proteases from unhatched louse eggs (sample 4) (FIG. 1A, lane 4). Hypochlorite was used to treat unhatched eggs prior to the collection of ESWs for all subsequent protease analyses.

Several distinct proteases were observed in the ESWs from hypochlorite treated eggs collected up to 2 hours post egg-hatching (sample 5) (FIG. 1B). Bands of protease activity were detected around 25-30 kDa, 50 kDa and there were a number of fainter bands detected above 50 kDa. These proteases were specifically associated with the lice eggs at the time of egg hatching and were termed egg shell washings (ESW).

FIG. 2: The proteases present in the louse ESWs were further characterised by their mechanistic class. Incubation with the metal chelating agents EDTA and 1,10-phenanthroline, to inhibit metalloproteases, resulted in a reduction in protease activity compared to the untreated controls (FIGS. 2A and 2B, respectively). In contrast, there was no apparent reduction in protease activity when the ESW were incubated with the serine/cysteine protease inhibitor PMSF (FIG. 2B), the cysteine protease inhibitor E-64 (FIG. 2B) or the aspartic protease inhibitor pepstatin (data not shown).

FIG. 3: shows a two-dimensional gelatin SDS-PAGE that was used to more accurately assess the number of protease species present in the louse ESWs. Each of the three main regions of protease activity in the one-dimensional gelatin SDS-PAGE (FIG. 1B) resolved to a number of distinct proteases present in the louse ESWs with activity in the 25-30 kDa molecular weight range resolved to at least seven distinct proteases with isoelectric points in the neutral to alkaline pH range, whereas the band of protease activity around 50 kDa resolved to at least eleven distinct protease regions with iso-electric points in the acidic to neutral pH region. At least five proteases with molecular weights above 75 kDa were also observed.

In order to further investigate the effect of 1,10-phenanthroline on the protease activity of ESWs the proteases were separated by two dimensional gel electrophoresis and the gel incubated in the presence of 10 mM 1,10-phenanthroline. The results from these studies confirmed the inhibitory effect of this metalloprotease inhibitor on the activity of the louse egg proteases. There was a general reduction in protease activity in the 25-30 kDa region and a clear reduction in the proteases present around the 50 kDa region and above 75 kDa (FIG. 3B).

FIG. 4: shows the effect of 1,10-phenanthroline on egg hatching in lice. Eggs were treated 5 days post laying and then hatching observed over time.

FIG. 5: shows the effect of Lannate®, containing methomyl, on egg hatching in Helicoverpa eggs. The ovicidal efficacy was assessed at 5 mM, 2.5 mM, 2.25 mM and 0.125 mM of methomyl.

FIG. 6: shows the effect of 2-acetyl-1-tetralone on egg hatching in Helicoverpa armigera eggs. The ovicidal efficacy was assessed at 2 mM and 1 mM 2-acetyl-1-tetralone.

FIG. 7: shows the effect of 2-acetyl-1-tetralone on egg hatching in Plutella eggs. The ovicidal activity was assessed at 2 mM, 1 mM, 0.5 mM and 0.1 mM.

FIG. 8: shows the effect of 5,5′-dimethyl-2,2′-dipyridyl on egg hatching in H. contortus eggs. The ovicidal efficacy was assessed at 180 μg/mL, 18 μg/mL, 1.8 μg/mL and 0.18 μg/mL.

FIG. 9: shows the effect of ivermectin on egg hatching in H. contortus eggs. The ovicidal efficacy was assessed at 200 μg/mL, 100 μg/mL, 50 μg/mL, 25 μg/mL and 12.5 μg/mL.

FIG. 10: shows the effect of 2-acetyl-1-tetralone on egg hatching in H. contortus eggs. The ovicidal efficacy was assessed at 110 μg/mL and 22 μg/mL.

### DESCRIPTION OF THE INVENTION

In one aspect of the invention there is provided a method of inhibiting a remodelling process in an inverstebrate by externally applying a pesticide composition substantially as described herein below. The remodelling process to be inhibited may be any process that is involved in the life-cycle of an invertebrate pest. As such, the invention contemplates inhibiting processes such as egg hatching, excystment, exsheathment, apolysis, ecdysis or metamorphosis. Without being limited to a given theory or mechanism of action, the invention may but need not necessarily involve the inhibition of a protease enzyme involved in such a remodelling process. It is known, for example, that protease enzymes are involved in hydrolysing proteins in eggs, sheaths, carapaces, exoskeletons, cysts, cocoons or ootheca, weakening the structure and at least partially allowing the invertebrate to free themselves from the structure. In some alternative methods of the invention it is contemplated that the remodelling process indirectly involves a protease enzyme, for example, a given protein or peptide may be required for the remodelling process such as a hormone that signals that the remodelling process should occur and the compositions of the invention are able to inhibit the production or processing of such a hormone. Preferably the protease enzyme is a metalloprotease enzyme.

The term “remodelling event” refers to an event in the life cycle of an invertebrate that alters the invertebrates' immediate environment or alters the invertebrates' physical form and facilitates progression of the organism from one life stage in the life cycle to the next life stage. Examples of remodelling events include egg hatching, excystment, apolysis of a cuticle or exoskeleton, ecdysis of a cuticle or exoskeleton and metamorphosis.

As used herein, “egg hatching” refers to the hatching of an invertebrate from a thin membrane egg where hatching is assisted by protease enzymes. Thin membrane eggs include those eggs that possess shells or cuticles comprising predominantly a protein matrix, with or without tanned proteins, are permeable to gas but essentially water impermeable, are generally non-mineralised are less than 20 mm in length and are not amenable to hatching solely by mechanical means, for example, by chewing or unassisted bursting.

As used herein, “excystment” refers to the emerging of an embryonic larval, or quiescent protozoa, taeniid, trematode or other species from an enclosed membranous sac or tissue cavity at some stage of its life cycle. These sacs may be formed in part or whole from proteinaceous material that must be remodelled in order for the invertebrate to emerge. The cysts may be formed inside the body of a host such as a human or an aquatic snail or directly be deposited into the environment. Excystment must occur at the right time and manner in order for the invertebrate to continue with subsequent stages of its life cycle.

As used herein, “exsheathment”, refers to a moulting process in worms such as nematodes. After hatching, a nematode goes through four larval stages before emerging as an immature adult. The larval stages are encased in a cuticle or sheath that has a protective role. The process involves two steps, the synthesis of a new cuticle and the exsheathment or shedding of the old cuticle. Exsheathment may also be essential for allowing infection of a new host. For example, exsheathment of the third larval stage of H. contortus in the rumen of a host results in infection of the host. Environmental conditions in the rumen activate secretory cells in the nematode to release hormones. The hormones act on excretory cells and stimulate the uptake of water, which in turn, activates enzymes which are released into the space between the new and old cuticle. The enzymes weaken the old cuticle which then breaks allowing the worm to free itself from the old cuticle.

As used herein, “apolysis” refers to the separation of the cuticle from the epidermis of an invertebrate. This separation allows the formation of a new cuticle without exposure to the environment. During this process, enzymes are secreted from the invertebrate that digest the inner layers of the cuticle.

As used herein, the term “ecdysis” refers to the shedding of an old cuticle. Ecdysis occurs after apolysis. After apolysis, moulting fluid containing inactive enzymes are secreted into the space between the epidermis and the old cuticle. The new cuticle is then formed. The enzymes in the moulting fluid are then activated and the lower regions of the old cuticle, the endocuticle and mesocuticle, are digested. The exocuticle and epicuticle of the old cuticle, which are not digested, are then shed.

As used herein, “metamorphosis” refers to the biological process in which some invertebrates, after hatching, undergo a conspicuous change in form or structure through cell growth and differentiation which is often accompanied by a change in habitat and/or behaviour. Metamorphosis usually proceeds in distinct stages, usually starting with a larvae or nymph, optionally passing through a pupa, and results in an adult. Metamorphosis of a nymph, generally having the form of an adult, may be marked by the development of wings. In contrast, other invertebrates may have larvae that differ substantially from the adult and pass through an inactive stage called a pupa, from which an adult emerges. Growth and metamorphosis are controlled by hormones produced by the invertebrate. A combination of hormones may be used, for example, secretion of ecdysone (a steroid) and juvenile hormone allows moulting and growth of a nymph or larva without maturation by metamorphosis to an adult. When juvenile hormone ceases to be produced metamorphosis proceeds.

The term “exposing an invertebrate” as used herein refers to exposing the invertebrate at any part of its life cycle including, but not limited to, an invertebrate egg, ootheca, a cyst, an invertebrate nymph, an invertebrate larva, an invertebrate instar, an invertebrate pupa, and any juvenile stage or adult stage of an invertebrate. The term “contacting” as used herein may refer to an external contacting of the pest with the composition of the invention, Alternatively, the pest can be contacted with the composition of the invention because the pest has ingested the composition. In yet another alternative, the pest is contacted with the invention because the host of the pest has ingested or been in contact with the composition and by being in physical contact with the host, the pest either ingests or is externally contacted with the compositions of the invention. It is note that the compositions and methods of the invention are employed to kill, inhibit or otherwise disrupt the life cycle stage that is exposed to the compositions of the invention. For example, where the composition is used to inhibit egg hatching, the composition is exposed directly to the invertebrate egg rather than being exposed to a different stage in the life cycle of the invertebrate. As such, in the methods of the invention, it is not necessary that the inhibitory compositions are ingested by the host or even the pest in order to have their inhibitory effects as is required, for example, in the methods described in U.S. Pat. Nos. 5,766,609 and 6,150,125. Rather the compositions and methods of the present invention are such that the compositions are simply contacted to the outside surface or environment of the invertebrate and act either by killing the invertebrate directly or act to retard, inhibit or otherwise prevent the invertebrate from progessing through to the next stage in its life cycle. Thus, in certain embodiments, it is contemplated that the methods of the invention are used to treat a pest infestation by killing, inhibiting or otherwise disrupting such an infestation by arresting and removing the infestion in the invertebrate life cycle stage at which the composition is applied. Therefore, in the present invention, there is provided a method of treating a flea infestation in a manner such that the flea does not necessarily ingest the compositions of the invention, whereas for nematodes and caterpillars the composition may be ingested. U.S. Pat. No. 5,766,609 on the otherhand requires that a flea must ingest certain compounds in order to inhibit proteases that form significant components of the flea midgut and thereby reduce the fecundity of the fleas through such ingestion of protease inhibitors. Thus, a key difference between the use of the compositions of the present invention and the methods taught by U.S. Pat. No. 5,766,609 is that the compositions of the present invention act directly on the animal contacted/fed/otherwise exposed the compositions, rather than the compositions having an effect on subsequent generations (for example, where flea eggs are exposed, there is a decrease in egg hatching while the fleas themselves may well remain unaffected).

The term “metal chelating agent” as used herein refers to a molecule having at least two polar atoms, such as nitrogen, oxygen, sulfur and phosphorus, that are situated in the molecule such that they are capable of simultaneously coordinating to a metal ion. The metal chelating agent also has a clogP value of /1 and ≦4 and/or a molar refractivity in the range of 40 to 90 cm³/mole. In some embodiments, the chelating agent has a clogP value of /1 and ≦3 and a molar refractivity in the range of 40 to 70 cm³/mole. In some embodiments the pLD₅₀ of the chelating agent is /2, preferably /3, more preferably /4. In some embodiments the association constant or LogKb of the metal chelating agents for zinc is >5.00.

The metal ions that are capable of being coordinated by the metal chelating agent are any metal ions that occur in metalloproteases, particularly metalloproteases that are involved in breaking down structures containing cross-linked proteins associated with eggs, sheaths, carapaces, cuticles, exoskeletons, cysts or ootheca and/or that facilitate the progression of the organism from one life stage to the next life stage. Such metal ions include divalent and trivalent metal ions, particularly divalent alkaline earth metal ions and divalent or trivalent transition metal ions. In some embodiments the metal ions that are capable of being coordinated are selected from Ca⁺⁺, Mg⁺⁺, Cu⁺⁺, Fe⁺⁺, Zn⁺⁺ and Fe⁺⁺⁺, especially Cu⁺⁺, Fe⁺⁺ and Zn⁺⁺, more especially Zn⁺⁺.

ClogP is a calculated prediction of a compound's logP value. The logP value of a compound, which is the logarithm of its partition coefficient between n-octanol and water [(log(C_{octanol}/C_{water})], is a well established measure of the compound's hydrophilicity. The clogP calculation is based on atom type and includes information relating to various atomic properties such as atomic number, ring membership, bond types with immediate neighbours and aromaticity state. ClogP may be calculated using a clogP program (Biobyte).

Molar refractivity is a measure of the volume occupied by an atom or group and depends on temperature, the index of refraction, pressure. Molar refractivity provides an indication of size of the molecule and the polarizability of the molecule. Molar refractivity was calculated using the CMR module (Calculated Molar Refractivity) from the ClogP software program (Biobyte).

Log LD₅₀ is obtained from the observed percentage ovicidal activity by conversion using a modified logit transformation. The observed percentage ovicidal activity values were transformed using the equation:

BA=log((5+%)/(105−%))

The additional 5% was used to allow a number to be calculated when 0% and 100% activity was observed. The BA is a crude correction to the log of the concentration at which the compounds were tested. Correlation between BA values and LD₅₀ allows the calculation of Log LD₅₀. A pLD₅₀ value less than 2 is considered inactive (Class 0), a pLD₅₀ value between 2 and 3 is considered weakly active (Class 1), a pLD₅₀ value between 3 and 4 is considered moderately active (Class 2) and a pLD₅₀ greater than 4 is considered strongly active (Class 3). In preferred embodiments, the pLD₅₀ value is greater than 2, especially greater than 3 and more especially greater than 4.

Without wishing to be bound by theory, it is believed that the metal chelating agents bind to and remove the metal ions required for metalloprotease activity rendering the protease inactive. This theory is supported by the addition of metal ions reversing the inhibitory effect of the metal chelating agent.

In some embodiments, the metal chelating agent is a compound of formula (I):

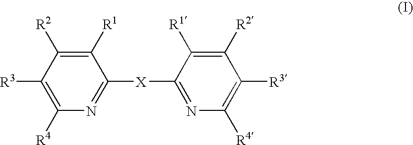

wherein X is selected from a covalent bond, —C(R⁵)₂—, —Z— or —C(R⁵)₂—Z—C(R⁵)₂—;

R¹ and R^{1′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R¹⁹ taken together are —C(R⁵)₂—, —C(R⁵)₂—C(R⁵)₂—, —CR⁵═CR⁵—, C(O), C(S) or NH;

R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or

R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;

each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, CO₂H, CO₂C₁₋₆alkyl, SO₃H, SO₃C₁₋₆alkyl, NH₂, NHC₁₋₆alkyl or N(C₁₋₆alkyl)₂;

each R⁶ is independently selected from hydrogen and halogen; and

Z is selected from a covalent bond, —NH—, —O—, —S—, —C(O)— and —C(S)—;

or a pharmaceutically, veterinary or agriculturally acceptable salt thereof.

Preferred compounds of formula (I) have at least one of the following features:

R¹ and R^{1′} are independently selected from C₁₋₆alkyl, a branched-chain C₁₋₆alkyl, C₂₋₆alkenyl, C₂₋₆alkynyl, hydroxy, C₁₋₆alkoxy, thiol, CO₂H, CO₂C₁₋₆alkyl, SO₃H, SO₃C₁₋₆alkyl, NH₂, NHC₁₋₆alkyl or N(C₁₋₆alkyl)₂, more preferably hydrogen or C₁-C₃alkyl, even more preferably hydrogen or methyl;

R² and R²; are independently hydrogen or C₁₋₃alkyl, more preferably hydrogen;

R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, C₁₋₆alkoxy, C₁₋₆alkylthiol or CO₂C₁₋₆alkyl, preferably hydrogen or C₁₋₃alkyl, more preferably hydrogen or methyl; or R³ and R⁴ and/or R^{3′} and R^{4′} taken together with the carbon atoms to which they are attached form a 5 or 6 membered carbocyclic or heterocyclic ring, preferably an aromatic ring;

each R⁵ is independently selected from hydrogen, C₁₋₆alkyl, a branched-chain C₁₋₆alkyl, C₂₋₆alkenyl, C₂₋₆alkynyl, C₁₋₆alkoxy, C₁₋₆alkylthiol or CO₂C₁₋₆alkyl, preferably hydrogen or C₁₋₃alkyl, more preferably hydrogen or methyl;

each R⁶ is independently hydrogen or fluorine, especially where each R⁶ is fluorine;

X is a covalent bond, —CH₂—Z—CH₂— or Z, preferably a covalent bond; and

Z is —NH—, —O— or —S—, preferably —NH—.

In some embodiments, the substituents R¹, R², R³, R⁴, R^{1′}, R^{2′}, R^{3′} and R^{4′} are electron-donating, or do not affect the electron density of the pyridyl ring.

Preferred compounds are biaryl compounds of formula (I):

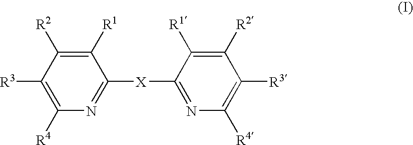

wherein X is selected from a covalent bond, —C(R⁵)₂—, —Z— or —C(R⁵)₂—Z—C(R⁵)₂—;

R¹ and R¹⁹ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂;

R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or

R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;

each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;

each R⁶ is independently selected from hydrogen and halogen; and

Z is selected from a covalent bond, —NH—, —O—, —S—, —C(O)— and —C(S)—;

or a pharmaceutically, veterinary or agriculturally acceptable salt thereof.

Preferred compounds of formula (I) include
2,2′-dipyridyl,
6,6′-dimethyl-2,2′-dipyridyl,
5,5′-dimethyl-2,2′-dipyridyl,
5,5′-diethyl-2,2′-dipyridyl,
4,4′-dimethyl-2,2′-dipyridyl,
2-(2-pyridinyl)quinoline,
2,2-dipyridylamine,
2,2′,6,2″-terpyridine

or a pharmaceutically, veterinary or agriculturally acceptable salt thereof.

In some embodiments, the preferred metal chelating agent is a compound of formula II:

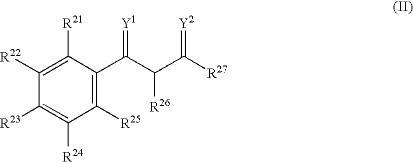

wherein Y¹ and Y² are independently selected form O, NR₂₈, or S;
R²¹, R²², R²³ and R²⁴ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl, N(C₁₋₆alkyl)₂ or a carbocyclic or heterocyclic ring; or
R²¹ and R²² or R²² and R²³ and R²⁴ taken together with the carbon atoms to which they are attached form a 5 or 6 membered carbocyclic or heterocyclic ring;
R²⁵ and R²⁶ are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, CN, C(R₂₉)₃, CO₂H, CO₂C₁₋₆alkyl, SO₃H, SO₃C₁₋₆alkyl, NH₂, NHC₁₋₆alkyl or N(C₁₋₆alkyl)₂; or
R²⁵ and R²⁶ together with the carbon atoms to which they are attached form a 5 or 6 membered carbocyclic or heterocyclic ring;
R²⁷ is C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, C(R²⁹)₂, N(R³⁰)₂, or a 5 or 6 membered carbocyclic ring or heterocyclic ring;
R²⁸ is hydrogen, C₁₋₆alkyl, or a branched-chain C₁₋₆alkyl;
R²⁹ is hydrogen or halogen; and
each R³⁰ is independently selected from hydrogen, C₁₋₆alkyl, a branched-chain C₁₋₆alkyl, C₂₋₆alkenyl, C₂₋₆alkynyl, a 5 or 6 membered carbocyclic ring or heterocyclic ring;
or a pharmaceutically, veterinary or agriculturally acceptable salt thereof.

Preferred compounds of formula (II) have at least one of the following features:
R²¹, R²², R²³ and R²⁴ are independently selected from hydrogen, C₁₋₆alkyl, a branched-chain C₁₋₆alkyl, C₂₋₆alkenyl, C₂₋₆alkynyl, halogen, NH₂, NHC₁₋₆alkyl, N(C₁₋₆alkyl)₂ or CN, preferably hydrogen or C₁₋₆alkyl, a branched-chain C₁₋₆alkyl, more preferably hydrogen or C₁₋₃alkyl, especially hydrogen or methyl;
R²⁵ and R²⁶ are independently selected from hydrogen, C₁₋₆alkyl, a branched-chain C₁₋₆alkyl, C₂₋₆alkenyl, C₂₋₆alkynyl, halogen, NH₂, NH(C₁₋₆alkyl), N(C₁₋₆alkyl)₂ or CN, preferably hydrogen or C₁₋₆alkyl, a branched-chain C₁₋₆alkyl, more preferably hydrogen or C₁₋₃alkyl, especially hydrogen or methyl; or
R²⁵ and R²⁶ taken together with the carbon atoms to which they are attached form a 6 membered carbocyclic or heterocyclic ring, especially a carbocyclic ring, more especially a 6 membered unsaturated carbocyclic ring;
R²⁷ is C₁₋₆alkyl, a branched-chain C₁₋₆alkyl, C₁₋₆alkenyl, C₁₋₆alkynyl, C(R²⁹)₃ or a 5 or 6 membered carbocyclic or heterocyclic ring; especially C₁₋₆alkyl, a branched-chain C₁₋₆alkyl or a 5 or 6 membered carbocyclic ring, preferably a C₁₋₃alkyl or a 6 membered carbocyclic ring; especially methyl or phenyl;
or a pharmaceutically, veterinary or agriculturally acceptable salt thereof.

Preferred compounds of formula (II) include:
dibenzoylmethane,
benzoylacetone, and
2-acetyl-1-tetralone,

or a pharmaceutically, veterinary or agriculturally acceptable salt thereof.

As used herein, the term “alkyl” refers to a straight-chain or branched saturated hydrocarbon group and may have a specified number of carbon atoms. For example, C₁-C₆ as in “C₁-C₆alkyl” includes groups having 1, 2, 3, 4, 5 or 6 carbons in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl and 3-ethylbutyl. C₁₋₆alkyl as used herein also includes branched chain C₁₋₆alkyl.

As used herein, the term “alkenyl” refers to a straight-chain or branched hydrocarbon group having one or more double bonds between carbon atoms and may have a specified number of carbon atoms. For example, C₂-C₆ as in “C₂-C₆alkenyl” includes groups having 2, 3, 4, 5 or 6 carbon atoms in a linear or branched arrangement. Examples of suitable alkenyl groups include, but are not limited to, ethenyl, propenyl, isopropenyl, butenyl, pentenyl and hexenyl.

As used herein, the term “alkynyl” refers to a straight-chain or branched hydrocarbon group having one or more triple bonds between carbon atoms, and may have a specified number of carbon atoms. For example, C₂-C₆ as in “C₂-C₆alkynyl” includes groups having 2, 3, 4, 5 or 6 carbon atoms in a linear or branched arrangement. Examples of suitable alkynyl groups include, but are not limited to, ethynyl, propynyl, butynyl, pentynyl and hexynyl.

As used herein the term “halo” or “halogen” refers to fluorine (fluoro), chlorine (chloro), bromine (bromo) and iodine (iodo).

The term “alkyloxy” or “alkoxy” as used herein represents an alkyl group as defined above attached through an oxygen bridge. Examples of suitable alkyloxy groups include, but are not limited to, methoxy, ethoxy, n-propyloxy, i-propyloxy, n-butyloxy, i-butyloxy, t-butyloxy, n-pentyloxy and n-hexyloxy.

The term “alkylthio” as used herein represents an alkyl group as defined above attached through a sulfur bridge. Examples of suitable alkylthio groups include, but are not limited to, methylthio, ethylthio, propylthio, i-propylthio, butylthio, i-butylthio, t-butylthio, pentylthio, hexylthio.

The term “carbocyclic ring” as used herein refers to a 3 to 10 membered ring or fused ring system, in which all of the atoms that form the ring are carbon atoms. The C₃₋₁₀carbocyclic ring may be saturated, unsaturated or aromatic. Examples of suitable carbocyclic rings include, but are not limited to, cyclopropyl, cyclobutyl, cyclopentyl, cyclopentenyl, cyclohexyl, cyclohexenyl, phenyl, naphthyl and tetrahydronaphthyl.

The term “heterocyclic ring” as used herein refers to a 3 to 10 membered ring or fused ring system in which at least one of the atoms that form the ring is a heteroatom. Preferably the heteroatom is selected from nitrogen, oxygen, sulfur and phosphorus. The C₃₋₁₀heterocyclic ring may be saturated, unsaturated or aromatic. Examples of suitable heterocyclic rings include, but are not limited to, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazoyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, aziridinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

As used herein, the term “aryl” is intended to mean any stable, monocyclic or bicyclic carbon ring of up to 6 atoms in each ring, wherein at least one ring is aromatic. Examples of such aryl groups include, but are not limited to, phenyl, naphthyl and tetrahydronaphthyl.

The term “heteroaryl” as used herein, represents a stable monocyclic or bicyclic ring of up to 6 atoms in each ring, wherein at least one ring is aromatic and at least one ring contains from 1 to 4 heteroatoms selected from the group consisting of O, N and S. Heteroaryl groups within the scope of this definition include, but are not limited to, acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline.

The compounds of the invention may be in the form of pharmaceutically, veterinary or agriculturally acceptable salts. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will also be recognised that many compounds of the invention possess asymmetric centres and are therefore capable of existing in more than one stereoisomeric form. The invention thus also relates to compounds in substantially pure isomeric form at one or more asymmetric centres e.g., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution.

A number of metal chelating agents and metalloprotease inhibitors useful in the present invention can be obtained commercially from specialty chemical companies. Those not commercially available can be synthesised from commercially available starting materials using reactions known to those skilled in the art.

For example, substituted 2,2′-bipyridyls and 1,10-phenanthrolines may be obtained from suitable halogenated 2,2′-bipyridyls or 1,10-phenanthrolines. For example, 2,2′-bipyridin-6,6′-dicarboxylic acid may be obtained from 6,6′-dibromo-2,2′-dipyridyl by halogen-metal exchange with butyl lithium, treatment with dry ice and acidification [Buhleier et. al., Chem. Ber., 1978, 111: 200-204]. Monosubstitution of a bipyridyl, for example with CH₂CHNH₂(CO₂H) at the 6 position, can be obtained by treatment of 6-methyl-2,2′-bipyridyl with N-bromosuccinimide followed by alkylation with N-protected-glycine ester. The protecting groups can then be removed by acid hydrolysis, (Imperiali B. and Fisher S. L., J. Org. Chem., 1992, 57: 757-759).

2,2′-Dipyridyls can undergo nucleophilic substitution at the C6 and C4 positions to introduce substituents. This reaction is more favorable when a halogenated dipyridyl is used as the starting material. For example an amine may be introduced at C6 and/or C6′ by using 6-mono or di-halogenated 2,2′-dipyridyl and reacting this starting material with ammonia.

Bipyridyl-sulfonic acids can be prepared from 2,2′-bipyridyl by heating with either oleum (a solution of sulfur trioxide in concentrated sulfuric acid) or mercury (II) sulfate/concentrated sulfuric acid at 300° C.

Unsymmetrically substituted bipyridyls can be obtained from symmetrical bipyridyls, for example, 6′-methyl-2,2′-bipyridyl-6-carboxylic acid can be prepared from 6,6′-dimethyl-2,2′-bipyridyl by oxidation with selenium dioxide followed by treatment with silver nitrate (Al-Saya et. al., European J. Org. Chem., 2004, 173-182).

Compounds of formula (II) may be prepared by reacting an appropriately substituted benzaldehyde and a ketone such as acetophenone or acetone using an aldol reaction then converting the resulting hydroxyketone to a 1,3-diketone as shown in Scheme 1:

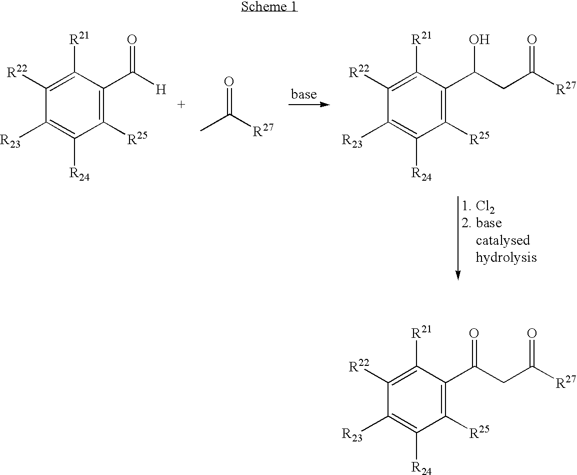

Compounds of formula (II) in the form of tetralones may be prepared using the Haworth reaction followed by α-substitution as shown in Scheme 2:

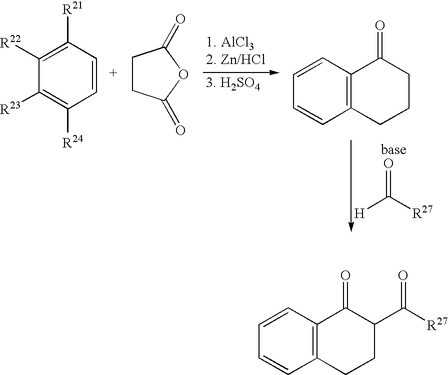

The invertebrates that are inhibited from undergoing remodelling events in the present invention are pests that internally or externally infest humans or animals, infest plants or infest property or a particular environment. For example, pests that internally infest humans or animals include, but are not limited to, nematodes, trematodes and cestodes, pests that externally infest humans or animals include, but are not limited to, lice, fleas, mites and scabies, pests that infest plants include, but are not limited to, moths, beetles, caterpillars butterflies and nematodes, pests that damage property include, but are not limited to, termites and pests that infest an environment include, but are not limited to, arachnids, cockroaches, centipedes, fleas and mites.

In another embodiment there is provided a method of treating or preventing a pest infestation of a host or environment comprising applying or administering to the host or environment an effective amount of at least one metal chelating agent, wherein the metal chelating agent has at least two polar atoms capable of simultaneously coordinating with a metal ion and
i) a clogP value of /1 and ≦4; and/or
ii) a molar refractivity in the range of 40 to 90 cm³/mole;

or a pharmaceutically, veterinary or agriculturally acceptable salt thereof.

The host treated by the methods of the invention may be selected from, but is not limited to, the group consisting of humans, sheep, cattle, horses, pigs, poultry, dogs and cats. The methods of treatment or prevention of the present invention may be applicable to plants and or other breeding, feeding or habitation sites of pests. Plants treated by the methods of the invention are preferably selected from, but are not limited to, the group consisting of cotton, oil seed or cereal grain crops such as canola, forestry crops such as trees, specimen plants such as trees, ornamental plants such as shrubs, flowers such as chrysanthemum, michaelmas daisy, geraniums and pinks, fruit trees such as apples, pears, plums, kiwifruit and citrus varieties for example, lemons, oranges, limes and grapefruit, cereal crops such as maize and sweet corn, vine crops such as grapes, root crops, pasture plants such as red and white clover, lucerne and lupins, and vegetables such as brassica crops, for example, broccoli and cauliflower, cabbage, tomatoes, zucchini, leeks, lettuce and beans as well as pulses such as navy beans, soybeans, mungbeans, pigeon pease and chickpeas and vine crops such as grapes.

The environment to be treated by the methods of the present invention includes the surroundings of an animal, human or plant that is or may become infested with a pest and includes but is not limited to soils surrounding plants or houses, gardens, lawns, kennels, barns or animal enclosures, carpets, clothing, bed linen and beds and the breeding sites of pests. The environment also includes property that may be damaged by a pest, for example buildings, furniture and wooden products that may be damaged or destroyed by termites.

Preferred pests that undergo remodelling events and may be controlled by the methods described include but are not limited to a species from a class, subclass or an order selected from the phylum Platheminthes such as the classes Cestoda and Trematoda, from the phylum Nematoda such as the classes Adenophoria or Secernentia, from the phylum Arthropoda such as the classes Crustacea, Arachnida, Insecta and Acarina.

From the class cestoda there are two orders namely Cestodaria and Eucestodia of which the cyclophyllideans are of the most importance to humans because they infect people and livestock. Two important tapeworms are the pork tapeworm, Taenia solium, and the beef tapeworms, T. saginata.

From the class trematoda the subclass, Digenea which includes the flukes. The flukes can be classified into two groups, on the basis of the system which they infect. Tissue flukes, are species which infect the bile ducts, lungs, or other biological tissues which includes the lung fluke, Paragonimus westermani, and the liver flukes, Clonorchis sinensis, Fasciola hepatica and Fasciola gigantica. The other group are known as blood flukes, and inhabit the blood in some stages of their life cycle. Blood flukes include various species of the genus Schistosoma.

Nematodes commonly parasitic on humans include whipworms, hookworms, pinworms, ascarids, and filarids. Within the nematode phylum is the class Adenophoria, and the subclass Enoplia that include the roundworms. Most nematodes in this subclass are free-living, but the group also includes the order Trichiurida, which includes the parasitic whipworms and trichina worms.

Also within the nematodes are the Secernentea, subclass Rhabditia that is mostly comprised of parasitic nematodes, though there are some free-living species as well. An important order is the Ascaridida, which includes worms that infect many land mammals and marine mammals. Important families within this order include Ascarididae, which includes the giant intestinal roundworm and related species and Toxocaridae, which includes parasites of canids, felids, and raccoons, but which can aberrantly parasitize humans and cause visceral larval migrans. Another important order is the Strongylida which includes the genus Metastrongylus a nematode of the family Metastrongylidae, usually found as lungworms in pigs and sometimes causing verminous bronchitis. The subfamily Strongylinae (large strongyles) and Cyathostominae, (small strongyles), are important nematodes of horses, while the family Trichostrongylidae contains a number of economically important intestinal parasites of sheep and cattle including Haemonchus contortus, H. placei, Teladorsagia circumcincta, Trichostrongylus colubriformis, and Cooperia spp.

In addition to the nematode parasites of mammalian hosts there are several groups of plant parasitic nematodes that can cause severe crop losses. The most common genera are Aphelenchoides (foliar nematodes), Meloidogyne (root-knot nematodes), Heterodera, Globodera (cyst nematodes), such as the potato nematode, Nacobhus, Pratylenchus (lesion nemtodes), Ditylenchus Xiphinema, Longidorus, Trichodorus. Several phytoparasitic nematode species cause histological damage to roots, including the formation of visible galls (Meloidgyne) which are useful characters for their diagnostic in the field. Some nematode species transmit plant viruses through their feeding activity in roots. One of these nematodes is Xiphinema index, vector of GFLV (Grapevine Fanleaf Virus), an important disease of grapes. Other nematodes attach bark and forest trees. The most important representative of this group is Bursaphelenchus xylophilus, the pine wood nematode, present in Asia and America and recently discovered in Europe.

From the crustacea, class Maxillopoda, subclass Maxillopoda that includes the fish lice.

From the Insecta, orders include: Lepidoptera, Hemiptera, Orthoptera, Psocoptera, Hymenoptera, Isoptera, Coleoptera, Dictyoptera, Thysanoptera, Homoptera , Diptera, Siphonaptera and Phthiraptera that comprises the Anoplura and Mallophaga.

Suitable pests that may be controlled using the methods of the present invention include:
(a) from the order of the lepidopterans (Lepidoptera), for example, Adoxophyes orana, Agrotis ypsilon, Agrotis segetum, Alabama argillacea, Anticarsia gemmatalis, Argyresthia conjugella, Autographa gamma, Cacoecia murinana, Capua reticulana, Choristoneura fumiferana, Chilo partellus, Choristoneura occidentalis, Chrysodexis Spp., Cirphis unipuncta, Cnaphalocrocis medinalis, Crocidolomia binotalis, Crocidolomia pavonana, Cydia pomonella, Dendrolimus pini, Diaphania nitidalis, Diatraea grandiosella, Earias insulana, Elasmopalpus lignosellus, Epiphyas postvittana (Walker), Eupoecilia ambiguella, Feltia subterranea, Grapholitha funebrana, Grapholitha molesta, Helicoverpa spp. such as Helicoverpa armigera, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera scitella, Lithocolletis blancardella, Lobesia botrana, Loxostege sticticalis, Lymantria dispar, Lymantria monacha, Lyonetia clerkella, Manduca sexta, Malacosoma neustria, Mamestra brassicae, Mocis repanda, Operophthera brumata, Orgyia pseudotsugata, Ostrinia nubilalis, Pandemis heparana, Panolis flammea, Pectinophora gossypiella, Phthorimaea operculella, Phyllocnistis citrella, Pieris brassicae, Pieris rapae, Plathypena scabra, Platynota stultana, Plutella xylostella, Prays cirri, Prays oleae, Prodenia sunia, Prodenia ornithogalli, Pseudoplusia includens, Rhyacionia frustrana, Scrobipalpula absoluta, Sesamia inferens, Sparganothis pilleriana, Spodoptera frugiperda, Spodoptera littoralis, Spodoptera litura, Syllepta derogata, Synanthedon myopaeformis, Thaumatopoea pityocampa, Tortrix viridana, Trichoplusia ni, Tryporyza incertulas, Zeiraphera canadensis; especially Heliothis spp., Helicoverpa Spp., Crocidolomia pavonana, Pieris rapae, Phthorimaea operculella, Chrysodexis Spp., and Plutella xylostella;
(b) from the order of the hemipterans (Hemiptera), for example, Aphis, Bemisia, Phorodon, Aeneolamia, Empoasca, Parkinsiella, Pyrilla, Aonidiella, Coccus, Pseudococcus, Helopeltis, Lygus, Dysdercus, Oxycarenus, Nezara, Aleyrodes, Triatoma, Psylla, Myzus, Megoura, Phylloxera, Adelges, Nilaparvata, Nephotettix or Cimwx spp.;
(c) from the order of the orthopterans (Orthoptera), for example, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Schistocerca americana, Schistocerca peregrina, Stauronotus maroccanus, Schistocerca gregaria;
(d) from the order of the psocopterans (Psocoptera), for example, Peripsocus spp.;
(e) from the order of the hymenopterans (Hymenoptera), for example, Athalia rosae, Atta cephalotes, Atta sexdens, Atta texana, Hoplocampa minuta, Hoplocampa testudinea, Iridomyrmes humilis, Iridomyrmex purpureus, Monomorium pharaonis, Solenopotes capillatus, Solenopsis geminata, Solenopsis invicta, Solenopsis richteri, Technomyrmex albipes;
(f) from the order of the termites (Isoptera), for example, Calotermes flavicollis, Coptotermes spp, Leucotermes flavipes, Macrotermes subhyalinus, Nasutitermes spp such as Nasutitermes walkeri, Odontotermes formosanus, Reticulitermes lucifugus, Termes natalensis;
(g) from the order of the beetles (Coleoptera), for example, Anthonomus grandis, Anthonomus pomorum, Apion vorax, Atomaria linearis, Blastophagus piniperda, Cassida nebulosa, Cerotoma trifurcata, Ceuthorhynchus assimilis, Ceuthorhynchus napi, Chaetocnema tibialis, Conoderus vespertinus, Crioceris asparagi, Dendroctonus refipennis, Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typo graphus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllopertha horticola, Phyllophaga sp., Phyllotreta chrysocephala, Phyllotreta nemorum, Phyllotreta striolata, Popillia japonica, Psylliodes napi, Scolytus intricatus, Sitona lineatus, Sitophilus granarius;
(h) from the order Dictyoptera, for example, from the families Polyphagidae, Bladberidae, Blattidae, Epilampridae, Chaetecsidae, Metallycidae, Mantoididae, Amorphoscelidae, Eremiaphilidae, Hymenopodidae, Mantidae and Empusidae;
(i) from the order of the thrips (Thysanoptera), for example, Frankliniella fusca, Frankliniella occidentalis, Frankliniella tritici, Haplothrips tritici, Heliothrips haemorrhoidalis, Scirtothrips citri, Thrips oryzae, Thrips palmi, Thrips tabaci;
(j) from the order of the homopterans (Homoptera), for example, Acyrthosiphon onobrychis, Acyrthosiphon pisum, Adelges laricis, Aonidiella aurantii, Aphidula nasturtii, Aphis fabae, Aphis gossypii, Aphis pomi, Aulacorthum solani, Bemisia tabaci, Brachycaudus cardui, Brevicoryne brassicae, Dalbulus maidis, Dreyfusia nordmannianae, Dreyfusia piceae, Dysaphis radicola, Empoasca fabae, Eriosoma lanigerum, Laodelphax striatella, Macrosiphum avenae, Macrosiphum euphorbiae, Macrosiphon rosae, Megoura viciae, Metopolophium dirhodum, Myzus persicae, Myzus cerasi, Nephotettix cincticeps, Nilaparvata lugens, Perkinsiella saccharicida, Phorodon humuli, Psylla mali, Psylla pini, Psylla pyricola, Rhopalosiphum maidis, Schizaphis graminum, Sitobion avenae, Sogatella furcifera, Toxoptera citricida, Trialeurodes abutilonea, Trialeurodes vaporariorum, Viteus vitifolii;
(k) from the order of the dipterans (Diptera), for example, Anastrepha ludens, Ceratitis capitata, Contarinia sorghicola, Dacus cucurbitae, Dacus oleae, Dasineura brassicae, Delia coarctata, Delia radicum, Hydrellia griseola, Hylemyia platura, Liriomyza sativae, Liriomyza trifolii, Lucilia Sp., Mayetiola destructor, Musca sp., Orseolia oryzae, Oscinella frit, Pegomya hyoscyami, Phorbia antiqua, Phorbia brassicae, Phorbia coarctata, Rhagoletis cerasi, Rhagoletis pomonella;
(l) from the order of Phthiraptera (Anaplura), for example, Pthirus pubis, Pediculus humanus capitus, Pediculus humanus humanus, the long nosed sucking louse, linognathus vituli, the short nosed sucking louse, Haematopinus eurystemus, the little blue louse, Solenopotes capillatus, the buffalo louse, Haematopinus tuberculatus, and the tail switch louse, Haematopinus quadripertusis, and from the Mallophaga, for example, from the genera Bovicola, such as Bovicola ovis or Bovicola bovis, Damalania, Trichodectus and Menopon; especially Bovicola ovis or Bovicola bovis;
(m) from the order of the siphonapterans (Siphonaptera), for example, Ctenocephalides or Pulex spp.
(n) from the order Blattodea, including Periplaneta Americana, Blattella germanica and Blattela asahinai;
(o) from the Dermaptera which are the earwigs;
(p) from the order Arachnida, for example, Ixodes holocyclus, Boophilus microplus, Rhipicephalus sanguineus, Sarcoptes scabiei var. humani, Sarcoptes scabiei canis, Sarcoptes scabiei suis, Sarcoptes scabiei bovis, Psoroptes ovis and Dermatophagoides spp., especially Sarcoptes scabiei var. humani, Sarcoptes scabiei canis, Sarcoptes scabiei suis, Sarcoptes scabiei bovis, Psoroptes ovis and Dermatophagoides spp.

Especially preferred pests that infest plants include Helicoverpa spp. such as Helicoverpa armigera, Helicoverpa Zea and Helicoverpa punctigera (Budworms), Crocidolomia pavonana (Cabbage cluster caterpillar), Pieris rapae (Cabbage white butterfly), Phthorimaea operculella (Potatoe moth), Chrsyodexis spp. (Tobacco loopers), Plutella xylostella (Diamondback moth) and Epiphyas postvittana (Walker) (Light brown apple moth), Cydia pomonella (Codling moth), Weevil spp, Aphelenchoides (foliar nematodes), Meloidogyne (root-knot nematodes), Heterodera, Globodera (cyst nematodes) such as the potato root nematode, Nacobbus, Pratylenchus (lesion nematodes), Ditylenchus, Xiphinema, Longidorus, Trichodorus Meloidogyne and Xiphinema index.

Especially preferred pests that infest domestic animals include Bovicola ovis (Sheep louse), Bovicola bovis, Haematopinus eurysternus (short-nosed cattle louse), Linognathus vituli (long nosed cattle louse), Solenopotes capillatus (tubercule-bearing louse), Sarcoptes scabiei canis (mange), Sarcoptes scabiei suis, Sarcoptes scabiei bovis, Psoroptes ovis, Haemonchus contortus, H. placei, Teladorsagia circumcincta, Trichostrongylus colubriformis, and Cooperia spp.

Especially preferred pests that infest humans include Pthirus pubis, Pediculus humanus capitus, Pediculus humanus humanus, Sarcoptes scabiei var. humani and Dermatophgoides spp.

In one embodiment, the pest which is prevented from undergoing a remodelling event by the present invention is selected from the group consisting of louse, flea, tick, fly, mite and other biting or blood-sucking pest eggs. In one embodiment, the pest egg is a louse egg, more preferably head louse egg. Lice are a parasite that feed on animal skin and blood and they deposit their digestive juices and faecal material into the skin. These materials, as well as the puncture wound itself, cause skin irritation and lesions from the resulting scratching, and can cause a serious infection with ganglionic inflammation. Lice are also vectors of certain diseases, such as exanthematic or epidemic typhus and recurrent fever. The adult female louse has a life span of about one month and can lay up to ten eggs a day. Lice that infect humans may include the species of crab louse (Pthirus pubis) and the separate species Pediculus humanus which is composed of two subspecies, Pediculus humanus capitis or head lice and Pediculus humanus humanus or clothing lice (Busvine, Antenna, 1993, 17: 196-201). The above subspecies of lice are closely related and are known to successfully interbreed in the laboratory situation (Busvine, Cutaneous Infestations and Insect Bites, 1985, 163-174).

The head louse Pediculus humanus var. capitis, is a host-specific ectoparasite that lives exclusively on human heads and feeds via sucking blood from the scalp. Following a blood meal, mature adult female lice will lay up to 10 eggs close to the scalp over a 24 hour period. The eggs are attached firmly to the hair shaft via a glue. Seven to ten days post laying depending on temperature and humidity, the eggs will hatch and the newly emerged nymphs begin to feed. The nymphs progress through three moults (1^{st} instar, 2^{nd} instar, 3^{rd} instar) with each moult taking between 3-5 days to complete. Following the final moult the adult male or female emerges with mating taking place as early as two days later. Within hours of feeding, eggs will be produced and the cycle continues. The entire life cycle from egg to egg takes approximately 20-30 days to complete depending on conditions of warmth and humidity. Following egg hatching the egg shell remains attached to the hair shaft and will gradually move away from the scalp as the hair lengthens. Hatched eggs (nits) are relatively easily detected due to their refractive nature appearing white under artificial light, in contrast unhatched eggs are a light pale brown in color enabling them to blend in to most hair colors and therefore making them more difficult to detect.

In another embodiment, the pest which is prevented from undergoing a remodelling event by the present invention is one that infests a plant host including the plant's roots. In a preferred embodiment, the pest is a budworm egg, a caterpillar egg, a butterfly, a moth or a root nematode. Caterpillars, butterflies, moths, soil nematodes and their larvae feed on valuable crop plants such as cotton, oil seed crops such as canola, ornamental plants, flowers, fruit trees, cereal crops, vine crops, root crops, pasture crops, tobacco, pulses and vegetables, especially Brassica crops such as cauliflower and broccoli, cotton, maize, sweetcorn, tomatoes, tobacco and pulses such as soybeans, navy beans, mungbeans, pigeon peas and chickpeas.

The diamondback moth (Plutella xylostella) larvae feed on all plants in the Brassica/cruciferae family, including canola and mustard, vegetable crops such as broccoli, cauliflower and cabbage and also on several greenhouse plants. Normally the diamondback moth takes about 32 days to develop from egg to adult. However, depending on food and weather conditions, a generation may take from 21 to 51 days to complete. Adult female moths lay an average of 160 eggs over a lifespan of about 16 days. A female will lay eggs at night and will lay the largest number of eggs the first night after emergence from the pupa. The eggs are small, spherical or oval and yellowish-white and are glued to the upper or lower surfaces of a leaf either singly or in groups of two or three. The eggs are usually laid along the veins of the leaf where the leaf surface is uneven. The eggs hatch in about five to six days. After hatching, the larvae burrow into the leaf and begin eating the leaf tissue internally. After about a week, the larvae exit from the leaf and feed externally. The larvae moult three times over 10 to 21 days and at maturity are about 12 mm long. The larvae pupate in delicate, open-mesh cocoons attached to the leaves and the pupal stage lasts from 5 to 15 days.

Budworms such as corn ear worm, tomato grub, tobacco budworm and cotton Bollworm are serious pests in a number of crops such as sunflowers, zucchini, beans, peppers, alfalfa, potatoes, leeks, cotton, maize, plums, citrus plants, tomatoes, tobacco and lettuce, and flowers such as geraniums and pinks. Budworms occur in many regions of the world and in temperate climates may have 2-3 generations per season with pupae overwintering in the soil. In tropical regions, the budworms may continue to be active year round. Eggs are small (˜0.5 mm in diameter) and dome shaped with a slightly flattened bottom. Eggs are usually laid singularly near buds or flowering parts or on leaves. An adult may lay 500-3000 eggs. The eggs hatch after only three days at 25° C. or longer at cooler temperatures, for example, 9 days at 17° C. The larval feeding period is about 19 to 26 days under favourable temperature and feeding conditions and when fully developed the larvae move to the soil to pupate. The pupal period generally lasts from 8 to 21 days although diapausing pupae can overwinter in soil in temperate regions.

In another embodiment, the pest that is prevented from undergoing a remodelling event by the present invention is one that internally infests a human or animal. In a preferred embodiment, the pest is a nematode, a trematode or a cestode. Nematodes (roundworms), trematodes and cestodes are flat worms and may cause significant damage to humans or agriculturally important animals such as sheep, cows, pigs and goats.

Haemonchus contortus, an intestinal parasite that infests sheep and goats, and adult male and female worms live in the abomasum or the true stomach of ruminant animals. The female worms deposit 5,000 to 10,000 eggs per day which are passed out of the host with the faeces. After hatching the first and second stage juveniles feed on bacteria. The third stage juveniles retain a cuticle as a sheath and the third stage juvenile is ingested by the host while grazing. The young sheathed worms pass into the host and exsheath before entering the abomasum. In the abomasum the exsheathed young worms burrow into the mucosa and feed on blood. Once adulthood is reached, mating occurs and further eggs are laid. The entire life cycle of this parasite takes approximately 21 days. Infested sheep can suffer ill thrift resulting in weight loss and in heavy infestations, anaemia can result, which left untreated may cause the death of the animal.

In one embodiment of the present invention, the methods and compositions are to treat or prevent external infestation of a human or animal by a pest or parasite that undergoes remodelling events, such as lice, fleas, mites or ticks, by inhibiting these remodelling events. The inhibition of remodelling events has the advantage of interrupting the life cycles and/or breeding cycles of the pest or parasite thereby controlling infestation.

In another embodiment, the methods and compositions are to treat or prevent internal infestation of a human or animal by a pest or parasite that undergoes remodelling events, such as nematodes and trematodes, by inhibiting transition from one stage of the life cycle of the pest or parasite to the next. The inhibition of remodelling events has the advantage of interrupting the life cycles and breeding cycles of the pest or parasite at a number of different points thereby controlling infestation.

In yet another embodiment, the methods and compositions are to treat or prevent infestation of an environment with a pest or parasite by inhibiting remodelling events of the pest or parasite.

For example, the eggs of pests or parasites may be laid in soil around a plant, in carpet or curtains in a house (eg: flea eggs), linen or mattresses of bedding (eg: dust mite eggs or bed bug eggs) or on or in the vicinity of wooden structures such as buildings or other wooden products (eg: termite eggs). The hatching of the eggs allows reinfestation of humans, animals or plants in the environment or damage to products in the environment. The inhibition of remodelling events has the advantage of interrupting the breeding cycles of the pest or parasite thereby controlling infestation. Furthermore, the prevention of reinfestation results in a reduction of the number of applications of pesticides required to control an infestation.

In yet another embodiment of the present invention, the methods and compositions of the invention are to treat or prevent larval infestation of plants by inhibiting remodelling events. The present applicants have identified metal chelating agents as effective agents for inhibiting remodelling events that affect both eggs and larvae that feed on commercially valuable plants. The use of metal chelating agents for inhibiting remodelling events has the advantage of inhibiting breeding cycles of organisms that produce larvae that feed on commercially valuable plants thereby controlling pest infestation of the commercially valuable plants.

The term “metalloprotease” as used herein is taken to refer to a protease involved in invertebrate remodelling events during one or more stages of a pest species development, wherein the protease has an active metal ion that acts as a catalyst. Preferably, the metalloprotease contains a zinc ion that participates in catalysis by polarizing a water molecule to attack a substrate-peptide bond. More preferably, the metalloprotease is sensitive to metal chelating agents that are capable of either directly or indirectly blocking their activity. The metalloprotease may be involved in inducing egg hatching by acting on the operculum of an egg to facilitate egg hatching or may reduce the strength of the egg shell allowing the nymph or larvae to break out of the shell during hatching. The metalloprotease may also be involved in facilitating the change from one larval or immature stage to a subsequent stage and also to the adult or mature form. The metalloprotease may be directly or indirectly involved in the remodelling events. Suitable metalloproteases involved in remodelling events can include endoproteases (enzymes that cleave within the peptide chain) and exoproteases (enzymes that cleave amino acid(s) from the termini of peptides). Exoproteases can further be categorised as carboxyproteases (which cleave amino acid(s) from the C terminus) or aminopeptidase (which cleave amino acids from the N terminus). Metallo-carboxyproteases require a bivalent cation (usually Zn²⁺) for activity, while aminopeptidases are generally classified according to their dependence on metal ions (Zn²⁺ or Mg²⁺). They exist in both free and membrane-bound forms and favour activity at high (8-10) pH. One method of detecting metalloproteases associated with egg hatching can involve collecting either the fluid surrounding the developing embryo at the time of egg hatching or by washing the empty egg shells shortly after egg hatching and analyzing the sample for the presence of proteases using gelatine substrate SDS-PAGE analysis. Having shown the presence of proteolytic activity from the sample it is then possible to incubate the sample in the presence of a metalloprotease inhibitor that has been identified as having the required arrangement of polar atoms and clogP values and/or molar refractivity and in some embodiments, a preference for chelating with zinc ions, and then reanalyze the treated sample to determine if the activity of the proteases extracted from the egg have been inhibited. Having shown inhibition of the activity of the metalloprotease(s) obtained from the hatched egg, it is then possible to expose unhatched eggs, for example, to the same inhibitor and assess whether inhibition of egg hatching occurs. Similar approaches can be made to determine metal chelating agents suitable for inhibiting other remodelling events such as apolysis, ecdysis exsheathment or metamorphosis. For example, fluid may be obtained from invertebrates undergoing apolysis, ecdysis or metamorphosis and the presence of proteases detected as described above. Suitable metal chelating agents may then be determined. Metalloproteases involved in egg hatching may also be identified by identification of a gene encoding a metalloprotease, silencing that gene and showing that the egg is unable to hatch by methods known to those skilled in the art.

The phrase “inhibiting remodelling events” as used herein is taken to mean the inhibition of protease enzymes involved in remodelling events that involve encasements of invertebrate multi-cellular organisms, for example, eggs, sheaths, carapaces, exoskeletons, cysts, cocoons or ootheca. In the present invention a particular life cycle stage of invertebrate pest is exposed to a metal chelating agent that is capable of preventing a remodelling event when compared to the same life cycle stage that is untreated. In the case of egg hatching this remodelling event may be characterised by the hatchflap or operculum of an egg opening and shortly thereafter the emergence of a larvae or nymph. In the case of lice, the head appears first followed by the thorax to which the legs are attached. Finally, the abdomen emerges and the nymph moves free from the egg. In the case of a moth or butterfly egg, the eggshell is weakened by the action of protease enzymes and the emerging larva breaks through the eggshell. Egg hatching is taken to exclude damage or accidental breakage of an eggshell.

Preferably, the metal chelating agent is a compound capable of inhibiting remodelling events when it is applied to a stage of the pests life cycle at any time between laying and throughout adults life.

The remodelling event preferably takes place in a pest present on, but not limited to, a host organism, such as on the skin, hair, coat or fleece of an animal or skin or hair such as head hair of a human. In alternative embodiments of the invention the remodelling event takes place in a pest present on host plants or in the roots of plants including cereal crops, fruit trees, cotton, oil seed crops, ornamental plants, flowers, vine crops, root crops, pasture plants and vegetables. In yet other embodiments, the remodelling event takes place in a pest that is present in an environment or breeding site, such as, but not limited to, houses and buildings, enclosures for domestic and farming animals, carpets, bedding such as sheets and blankets, curtains and furniture. In yet other embodiments, the remodelling event may take place in a pest that is inside a host, such as, but not limited to, humans, domestic and farming animals.

According to the present invention, the pest may be exposed to a metal chelating agent by any suitable means. A person skilled in the art will appreciate that these means may vary widely, depending upon whether the chelating agent is to be applied to a host, such as a plant or applied or administered to an animal including a human, or applied to various environments of other breeding sites, and depending on the nature and type of pest targeted. Suitable means for exposing the pest present on animals to metal chelating agents, include, but are not limited to, direct topical application, such as by dipping or spraying, implants, delayed release formulations or devices, or orally. Where the invention is applied to humans, formulations suitable for topical application include but are not limited to sprays, aerosols, shampoos, mousses, creams and lotions, and formulations suitable for internal application include but are not limited to tablets, capsules or liquid formulations. In some situations parenteral administration by injection may be the most suitable means of treatment for humans or animals. Where the metal chelating agent is to be applied to plants, suitable means include but are not limited to sprays, dusts including wettable powders, wettable granules and suspension concentrates, pellets, liquids including micro-encapsulations and aerosols. The method of the invention also encompasses the concurrent or successive use of two or more metal chelating agents or the use of one or more metal chelating agents in conjunction concurrently or successively with other known agents that control pests.

In yet another aspect of the invention, the methods and compositions may include other pesticides that control hatching, larvae, nymphs or adult pests. For example, suitable pesticides which may be used in conjunction, either simultaneously, separately or sequentially, with the metal chelating agents of the present invention include macrocyclic lactones such as spinosad, botanical insecticides, carbamate insecticides, dessicant insecticides, dintrophenol insecticides, fluorine insecticides, formamidine insecticides such as armitraz, fumigant insecticides, inorganic insecticides, insect growth regulators, (including chitin synthesis inhibitors, juvenile hormone mimics, juvenile hormones, moulting hormone agonists, moulting hormone antagonists, moulting hormones, moulting inhibitors), nicotinoid insecticides, organochlorine insecticides, organophosphorus insecticides, heterocyclic organothiophosphate insecticides, phenyl organothiophosphate insecticides, phosphonate insecticides, phosphonothioate insecticides, phosphoramidate insecticides, phosphoramidothiate insecticides, phosphorodiamide insecticides, oxadiazine insecticides, phthalimide insecticides, pyrazole insecticides, pyrethroid insecticides, pyrimidinamine insecticides, pyrrol insecticides, tetronic acid insecticides, thiourea insecticides and urea insecticides including agents described in EP 0191236, U.S. Pat. No. 5,288,483 and U.S. Pat. No. 6,727,228. Other useful insecticides include dimethicone copolyols, such as those described in U.S. Pat. No. 6,663,876 and U.S. Pat. No. 6,607,716, which have low toxicity. Useful nemiticides that may be used include Oxfendazole, Abendazole, Mebendazole/closantel, Fenbendazole and triclabendazole. In terms of nematicides, oxamyl and fenamiphos are two compounds that are used to control these organisms in the soil. For treating trematode infections compounds such as Oxfendazole, Albendazole, Mebendazole/closantel, Fenbendazole, and triclabendazole. Trematode and cestode infections can also be treated with Praziquantel.

The metal chelating agent may be applied to the hair or skin of a host when the host is a human or animal, preferably in a region that is infested with a pest. The infestation may be due to pests selected from the group consisting of lice, fleas, ticks, flies, mites and other biting or blood-sucking pests, and combinations thereof. The metal chelating agent may be applied topically in the form of ointments, aqueous compositions including solutions and suspensions, creams, lotions, aerosol sprays or dusting powders. When the pest internally infests the human or animal, the metal chelating agent may be applied or administered internally, for example, in the form of a tablet, capsule or ingestable liquid formulation. When the host is a plant, the pest infestation is preferably due to pests selected from, caterpillars, butterflies, moths or nematodes. The metal chelating agent may be applied topically, for example, in the form of a spray or dust. When the infestation is in the environment, such as a termite infestation, the metal chelating agent may be applied in a formulation such as a spray, fumigant or dust.

The term “effective amount” means a concentration of at least one metal chelating agent sufficient to provide treatment or prevention of a pest infestation in a host or in an environment. The effective amount of a metal chelating agent used in the methods of the present invention may vary depending on the host and the type and level of infestation. In one embodiment, the metal chelating agent is applied to the scalp of a person suffering from head lice infestation and are left on the treated person for a period of time to prevent hatching of the louse eggs. Preferably the period of time is between 5 and 15 minutes. The metal chelating agent is preferably used at a concentration of between about 0.0001 mM to 1M, preferably 0.01 mM and 100 mM, more preferably in the range of 0.1 mM and 100 mM. The effective amount depends on the metal chelating agent used. However, some dipyridyl compounds may suitably be applied in the range of 5 mM to 100 mM, especially at a level of about 50 mM. Suitable amounts of compounds of formula (II), such as tetralone compounds, may be applied at a level in the range of 0.5 mM to 100 mM, especially 1 mM to 50 mM. Since a significant number of mammalian proteases require zinc for their activity and may be affected by metal chelating agents, it would be necessary to ensure that the metal chelating agent was used in a safe and effective amount and is preferably specifically targeted to a specific remodelling event, such as egg hatching, apolysis, ecdysis, exsheathment or metamorphosis.

In another embodiment, the metal chelating agent is applied to a commercially valuable plant to prevent remodelling events occurring in a pest that are involved in, for example, egg hatching or moulting. The metal chelating agent may be applied directly or indirectly to pests which are present in the ground or on the leaves, buds, stems, flowers or fruit of a plant by spray application, brushing on or dusting. Suitable compositions include emulsifiable concentrates, directly sprayable or dilutable solutions, dilute emulsions, wettable powders, soluble powders, dusts or granules. The metal chelating agent is preferably used at a concentration of between about 0.0001 mM to 1M, preferably 0.01 mM and 100 mM, more preferably in the range of 0.1 mM and 30 mM. The effective amount depends on the metal chelating agent used. However, some dipyridyl compounds may suitably be applied in the range of 5 mM to 15 mM, especially at a level of about 10 mM. Suitable amounts of compounds of formula (II) include, but are not limited to, the range of 0.1 mM to 20 mM, especially 1.0 mM to 15 mM.

The host treated by the methods of the invention may be selected from, but is not limited to, the group consisting of humans, sheep, cattle, horses, pigs, poultry, dogs and cats. The methods of treatment or prevention of the present invention may be applicable to plants and or other breeding sites of pests. Plants or their roots treated by the methods of the invention are preferably selected from the group consisting of cotton, oil seed crops such as canola, ornamental plants such as shrubs, flowers such as chrysanthemum, michaelmas daisy, geraniums and pinks, fruit trees such as apples, pears, plums, kiwifruit, currants and citrus varieties for example, lemons, oranges, limes and grapefruit, cereal crops such as maize and sweetcorn, vine crops such as grapes, root crops, pasture plants such as red and white clover, lucerne and lupins, and vegetables such as brassica crops, for example, broccoli and cauliflower, cabbage, tomatoes, zucchini, leeks, lettuce and beans as well as pulses such as navy beans, soybeans, mungbeans, pigeon peas and chickpeas.

The compositions of the present invention may be formulated as solutions and emulsions. Suitable excipients, such as emulsifiers, surfactants, stabilizers, dyes, penetration enhancers and anti-oxidants may also be present in the compositions. Suitable carriers that may be added in the compositions can include, water, salt solutions, alcohols, polyethylene glycols, gelatine, lactose, magnesium sterate and silicic acid. The compositions may include sterile and non-sterile aqueous solutions. In one embodiment, the compositions are in a soluble form and the metal chelating agent is diluted in a soluble sterile buffered saline or water solution. The compositions can also be formulated as suspensions in aqueous, non-aqueous or mixed media. Aqueous suspensions may further contain substances that increase the viscosity of the suspension and may also contain stabilizers. The solutions may also contain buffers, diluents and other suitable additives. The compositions can include other adjunct components that are compatible with the activity of the metal chelating agent. The compositions of the present invention may be formulated and used as foams, emulsions, microemulsions, shampoos, mousses, creams and jellies. The formulations of the above compositions described would be known to those skilled in the field of pesticides.

The active ingredients according to the invention can be used for inhibiting remodelling events that occur in pests on plants or in their roots, mainly on crops of useful plants and ornamentals in agriculture, in horticulture and in silviculture, or on parts of such plants, such as fruits, flowers, foliage, stalks, tubers or roots, and in some cases even parts of plants which are formed at a later point in time are afforded protection against these pests. In these compositions, the active ingredient is employed together with at least one of the auxiliaries conventionally used in the art of formulation, such as extenders, eg solvents or solid carriers, or such as surface-active compounds (surfactants).

Examples of suitable solvents are: non-hydrogenated or partially hydrogenated aromatic hydrocarbons, preferably the fractions C₈-C₁₂ of alkylbenzenes, such as xylene mixtures, alkylated naphthalenes or tetrahydronaphthalene, aliphatic or cycloaliphatic hydrocarbons such as paraffins or cyclohexane, alcohols such as methanol, ethanol, propanol or butanol, glycols and their ethers and esters such as propylene glycol, dipropylene glycol ether, hexylene glycol, ethylene glycol, diethoxy glycol, ethylene glycol monomethyl ether or ethylene glycol monoethyl ether, ketones such as cyclohexanone, isophorone or diacetone alcohol, strongly polar solvents such as N-methylpyrrolid-2-one, N-methyl-pyrrolidine, dimethyl sulfoxide or N,N-dimethylformamide, water, free or epoxidized rapeseed, castor, coconut or soya oil, and silicone oils.

Solid carriers which are used for example for dusts and dispersible powders are, as a rule, ground natural minerals, such as calcite, talc, kaolin, montmorillonite or attapulgite. To improve the physical properties, it is also possible to add highly-disperse silicas or highly-disperse absorptive polymers. Suitable particulate adsorptive carriers for granules are porous types, such as pumice, brick grit, sepiolite or bentonite, and suitable non-sorptive carrier materials are calcite or sand. Moreover, a large number of granulated materials of inorganic or organic nature can be used, in particular dolomite or comminuted plant residues.

Suitable surface-active compounds are, depending on the nature of the active ingredient to be formulated, non-ionic, cationic and/or anionic surfactants or surfactant mixtures which have good emulsifying, dispersing and wetting properties. The surfactants listed below are only to be considered as examples; many more surfactants conventionally used in the art of formulation and suitable in accordance with the invention are described in the relevant literature.

Suitable non-ionic surfactants are primarily polyglycol ether derivatives of aliphatic or cycloaliphatic alcohols, of saturated or unsaturated fatty acids and alkylphenols which can contain 3 to 30 glycol ether groups and 8 to 20 carbon atoms in the (aliphatic) hydrocarbon radical and 6 to 18 carbon atoms in the alkyl radical of the alkylphenols. Also suitable are water-soluble polyethylene oxide adducts with polypropylene glycol, ethylenediaminopolypropylene glycol and alkylpolypropylene glycol having 1 to 10 carbons in the alkyl chain and 20 to 250 ethylene glycol ether and 10 to 100 propylene glycol ether groups. The above-mentioned compounds normally contain 1 to 5 ethylene glycol units per propylene glycol unit. Examples which may be mentioned are nonylphenylpolyethoxyethanols, castor oil polyglycol ethers, polypropylene/polyethylene oxide adducts, tributylphenoxypolyethoxyethanol, polyethylene glycol and octylphenoxypolyethoxyethanol. Also suitable are fatty acid esters of polyoxyethylene sorbitan, such as polyoxyethylene sorbitan trioleate.

The cationic surfactants are mainly quaternary ammonium salts which have, as substituents, at least one alkyl radical of 8 to 22 carbon atoms and, as further substituents, lower alkyl, benzyl or lower hydroxyalkyl radicals which may be halogenated. The salts are preferably in the form of halides, methylsulfates or ethylsulfates. Examples are stearyltrimethylammonium chloride and benzyldi(2-chloroethyl)ethylammonium bromide.

Suitable anionic surfactants can be both water-soluble soaps and water-soluble synthetic surface-active compounds. Soaps which are suitable are the alkali metal salts, alkaline earth metal salts and unsubstituted or substituted ammonium salts of higher fatty acids (C₁₀-C₂₂), such as the sodium or potassium salts of oleic or stearic acid, or of natural fatty acid mixtures which can be obtained, for example, from coconut or tall oil; or fatty acid methyltaurinates. However, synthetic surfactants, in particular fatty sulfonates, fatty sulfates, sulfonated benzimidazole derivatives or alkylarylsulfonates, are used more frequently. As a rule, the fatty sulfonates and fatty sulfates exist as alkali metal salts, alkaline earth metal salts or unsubstituted or substituted ammonium salts and generally have an alkyl radical of 8 to 22 carbon atoms, alkyl also including the alkyl moiety of acyl radicals. Examples of fatty sulfonates and fatty sulfates include the sodium or calcium salt of lignosulfonic acid, of the dodecylsulfuric ester or of a fatty alcohol sulfate mixture prepared with natural fatty acids. This group also includes the salts of the sulfuric esters and sulfonic acids of fatty alcohol/ethylene oxide adducts. The sulfonated benzimidazole derivatives preferably contain 2 sulfo groups and one fatty acid radical having approximately 8 to 22 carbon atoms. Examples of alkylarylsulfonates are the sodium, calcium or triethanolammonium salts of dodecylbenzenesulfonic acid, of dibutylnaphthalenesulfonic acid or of a naphthalenesulfonic acid/formaldehyde condensate. Also suitable are corresponding phosphates, such as salts of the phosphoric ester of a p-nonylphenol(4-14)ethylene oxide adduct, or phospholipids.

In a preferred embodiment, the composition comprises a metal chelating agent at a concentration of about 0.0001 mM to 1M, preferably between 0.1 mM to 100 mM, more preferably in the range of 0.1 mM to 50 mM. Compositions containing some metal chelating agents, for example, the compounds of formula I, may preferably contain between 5 and 50 mM of compound, especially at a level of about 30 mM. Compositions containing compounds of formula (II) may preferably contain between 0.1 mM to 100 mM, especially 1.0 mM to 50 mM.

A compound which inhibits egg hatching remodelling events in a pest, may be identified using a method comprising assessing the clogP value and/or molar refractivity of the compound and/or the ability of the compound to bind zinc and/or inhibit a metalloprotease involved in the remodelling event.

In a further aspect of the invention, there is provided a method of selecting a chelating agent as a candaidate inhibitor of invertebrate remodelling events from a collection of metal chelating agents said method comprising selecting metal chelating agents that have at least two polar atoms capable of simultaneously coordinating with a metal ion and
(i) a clogP value of /1 and ≦4; and/or
(ii) a molar refractivity in the range of 40 to 90 cm³/mole;

The clogP of a metal chelating agent may be calculated from its logP value using a clogP program, for example, the program provided by Biobythe. LogP values may be obtained from the literature or may be calculated from a measured partition co-efficient between n-octanol and water.

The molar refractivity of a metal chelating agent may be calculated using the CMR (calculated molar refractivity) software program from Biobythe.

In some embodiments, the metal chelating agent is selected to inhibit a zinc-metalloprotease enzyme involved in a invertebrate remodelling event. In such cases the chelating agent is further accessed for its ability to bind zinc ions. The ability of a metal chelating agent to bind zinc ions may be determined by determining the association constant (logKb) of the metal chelating agents for zinc. The association constant may be determined from the literature using methods know to those skilled in the art. In preferred embodiments in which the metalloprotease enzyme is a zinc-metalloprotease enzyme, metal chelating agents having an association constant for zinc of greater than 5.0 are selected.

A similar procedure may be followed if the metalloprotease to be inhibited includes a metal ion other than zinc, for example, Mg⁺⁺, Cu⁺⁺ or Fe⁺⁺. The association constant of the metal chelating agent for that metal ion may be assessed and metal chelating agents having the greatest association constants selected.

Identification of suitable metal chelating agents may further comprise testing the compound in a biological assay. A suitable biological assay preferably comprises exposing a control sample of pests in which the remodelling event may occur to a control buffer solution or control formulation whilst at the same time exposing a test sample of pests in which the remodelling even may occur to a solution or formulation comprising a test compound.

A compound that is effective in inhibiting a remodelling event in a pest is identified when the remodelling event is observed in the pests of the control sample or formulation and the remodelling event is not observed in the test sample of pests. In the biological assay of the present invention, the remodelling event may occur in a pest selected from the group consisting of louse, flea, tick, fly, mite and other biting or blood-sucking pests and further includes pests that live inside a mammalian host such as trematodes, nematodes and cestodes. In the biological assay of the present invention the remodelling event may occur in pests which infest plants such as caterpillars, moths, butterflies and soil nematodes. Alternatively, the remodelling event occurs in a pest that infests an environment, such as a termite egg or house dust mite egg.

The control buffer solution may include, but is not limited to, sterile phosphate buffered saline or water or an organic solvent. The compound tested is preferably a metal chelating agent. In an example of a biological egg hatching assay egg hatching is observed when the hatchflap or operculum of the egg opens and shortly thereafter the larvae or nymph begins to emerge. In the case of lice, the head appears first followed by the thorax to which the legs are attached. Finally, the abdomen comes out and the nymph moves free from the egg. In the case of head lice, the eggshell then remains cemented to the hair shaft. A metal chelating agent test compound may be identified as suitable for use in the invention if the eggs exposed to the control buffer display a high level, for example 70-100%, of hatching whereas the egg exposed to a test metal chelating agent display a low level, for example 0-30%, egg hatching, especially where 100% inhibition of egg hatching occurs.

In preferred embodiments, the pLD₅₀ of the selected metal chelating agent is greater than 2, preferably greater than 3, and more especially greater than 4.

Other similar biological assays may be used to assess the activity of selected metal chelating agents in inhibiting other remodelling events, such as excystment, exsheathment, apolysis, ecdysis or metamorphosis. For example, an invertebrate at a particular life stage, for example, a cyst, a larvae, a cocoon, a pupa, a nymph or an adult may be exposed to a test metal chelating agent in a carrier and the occurrence of a remodelling event such as excystment, exsheathment, apolysis, ecdysis or metamorphosis observed and compared to a control group of invertebrates exposed to carrier in the absence of test metal chelating agent.

In another aspect of the invention there is provided a use of at least one metal chelating agent in the manufacture of a composition for inhibiting a protease enzyme involved in invertebrate remodelling events or for treating or preventing pest infestation, wherein the at least one chelating agent has at least two polar atoms capable of simultaneously coordinating with a metal ion and
(i) a clogP value of /1 and ≦4; and/or
(ii) a molar refractivity in the range of 40 to 90 cm³/mole;
or a pharmaceutically, veterinary or agriculturally acceptable salt thereof.

In one embodiment, the pest is one infesting a plant host. In another embodiment, the pest is one infesting a domesticated animal. In yet another embodiment, the pest is one infesting a human. In a further embodiment, the pest is one infesting an environment.

Also encompassed by the present invention are agents comprising at least one metal chelating agent as described herein, for inhibiting a protease enzyme involved in invertebrate remodelling events or for treating or preventing pest infestation.

In some aspects of the invention it will be possible to identify additional pesticide agents for inhibiting invertebrate infestation of a host. As noted above compounds of Formula I and II have been found to be particularly useful in inhibiting various processes in invertebrate remodelling and/or invertebrate metabolic processes. The strategy of rational drug design can be used to identify specific such inhibitors. It is now established in the present invention that compounds of Formula I and II have useful properties in inhibiting invertebrate proteases. The drug design strategies may be created in which each of the R groups in the core structure of Formula I and Formula II is separately and individually fixed and the efficacy of the resulting agent in a given assay is determined. The structure of Formula I and Formula II that specifically interacts with the for example, the protease can be modeled using computational tools. These tools can allow a drug molecule to be constructed within the biomolecule using knowledge of its structure and the nature of its active site.

The compounds tested for efficacy as pesticides may be part of a set or library of compounds, which may be a diverse set or library or a focused set or library, as will be clear to the skilled person. The libraries that may be used for such screening can be prepared using combinatorial chemical processes known in the art or conventional means for chemical synthesis. Collections of compounds of the formula (I) and/or formula (II) which can be synthesized manually or in a semiautomated or fully automated manner. In this case, it is possible, for example, to automate the procedure for the production of such compounds, work-up or purification of the products or of the intermediates generally as described in, for example, by S. H. DeWitt in “Annual Reports in Combinatorial Chemistry and Molecular Diversity: Automated Synthesis”, Volume 1, Verlag Escom 1997, pages 69 to 77. In addition, compounds of the formula (I) and/or formula (II) may be prepared in part or fully by solid-phase-supported methods. For this purpose, individual intermediate steps or all intermediate steps of the synthesis or of a synthesis adapted to suit the procedure in question are bound to a synthetic resin. Solid-phase-supported synthesis methods are described extensively in the specialist literature, for example Barry A. Bunin in “The Combinatorial Index”, Academic Press, 1998.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In some specific embodiment, it is noted that the compounds used in the invention are not bestatin. The compound used in the invention is not 1,10-phenanthroline. In other embodiments, the compound used in the invention is not 2,2′-bipyridine.

The invention will hereinafter be described by way of the following non-limiting Figures and Examples.

### EXAMPLES

### Example 1

### Assessment of the Mechanism of the Remodelling Event Associated with Lice Egg Hatching

The mechanism of lice egg hatching was assessed under a dissecting microscope. Female clothing lice were fed for half an hour on a rabbit before being transferred to a petri dish containing human hair. The petri dish was then placed in an incubator at 32° C.; 32% relative humidity. Within 5 hours of feeding the female lice begin to lay their eggs. Each female lays up to 10 eggs at a sitting. The eggs develop over the next 7-9 days. Within the last 12 hrs prior to hatching the following changes were observed. The eyes of the developing embryo could be clearly detected inside the egg with the developing embryo orientated so that it has its head is adjacent to the hatch flap or operculum. The embryo can be observed moving within the egg. Hatching takes place when the operculum opens and shortly thereafter the embryo begins to emerge. The head appears first followed by the thorax to which the legs are attached. Finally, the abdomen comes out and the nymph moves free from the egg that remains cemented to the hair. There are no obvious structures associated with the head of the newly emerged nymph visible under light microscopy, that would facilitate hatching (ie no egg tooth is present). This observation suggests that while physical movement of the nymph within the egg probably contributes to egg hatching, other specific biochemical events are involved.

### Example 2

### Detection of Protease Activity in Lice Egg Extracts

Within 12 hours of hatching 50 body lice eggs (Pediculus humanus humanus) were removed from the hair and placed in a 1 mL eppendorf tube. 20 μL of distilled water was added to the unhatched eggs and the preparation incubated for 30 minutes at 32° C. The 20 μL was recovered, freeze dried and stored at −70° C. This sample was referred to as sample 1. A number of other samples were also collected as described. Sample 2 was collected by removing the unhatched louse eggs from four hairs that were approximately 3 cm long, cutting the hair into 0.5 cm lengths, and placing them into a microfuge tube containing 20 μl of distilled water and incubating at 32° C. for 30 minutes. Sample 3 was collected as for sample 2, but the hair was placed in a tube containing 10 mL of 1% sodium hypochlorite for 1 minute followed by five 1 minute washes in 25 mL of distilled water to remove the sodium hypochlorite before being incubated in a microfuge tube containing 20 μl of distilled water and incubated as for Sample 2. Sample 4 was collected from unhatched eggs which were removed from the hair and washed with 1% sodium hypochlorite and incubated in 20 μl of distilled water in the same manner as the hair in Sample 3. Finally, Sample 5 was collected from eggs that were within 24 hrs of hatching which were washed with 1% sodium hypochlorite, then returned to the incubator at 32° C. until they hatched, the empty egg shells collected 0-2 hrs after egg-hatching, placed in a 1 mL microfuge tube containing 20 μl of distilled water and incubated as for Samples 1-4. For all samples 1-5 the 20 μl of fluid was recovered, freeze-dried and stored at −70° C. The washings recovered from these freshly hatched egg shells are referred to as egg-shell-washings (ESWs). In order to look for the presence of proteases present in these different samples, the freeze-dried samples were resuspended in 15 μL of non-reducing SDS sample buffer, centrifuged at 10,000 g for 2 minutes and the entire 15 μL loaded on to 10% gelatine substrate SDS-PAGE gels. Gels were run at 4° C. for 10 minutes at 10 mA followed by a further 25 minutes at 15 mA per gel. They were then incubated for 2×20 minutes in a 2.5% Triton-X 100 solution followed by a three hour incubation in 0.1M Tris/HCl containing 1 mM CaCl₂ pH 8.0. Activity was detected as clear areas on the gel the result of protease activity degrading the gelatine within the gel.

Protease activity in the ESWs from louse eggs was also examined using two-dimensional gel electrophoresis. It was necessary to collect large numbers of freshly hatched egg shells. Following egglaying onto pieces of cloth, adult female lice were removed and the cloth with the eggs washed with 1% sodium hypochlorite as for collection of ESWs. The eggs were then returned to the incubator and permitted to hatch. Typically 100 to 500 hatched egg shells were collected (0-2 hours post hatching), placed in a microfuge tube containing 200 μl of distilled water, incubated and sample treated as described above. For analysis, ESWs were resuspended in rehydration buffer (8 M Urea, 2% 3-[(3-cholamidopropyl) dimethylammonio]-1-propanesulfonate 2% Immobilised pH gradient buffer 3-10 (Amersham Phamacia Biotech, Uppsala, Sweden)) and used to rehydrate 7 cm Immobiline Dry strips pH 3-10 (Amersham Bioscience) overnight. Strips were transferred to the Multiphor II (Pharmacia, Sweden) apparatus, electrophoresed in the first dimension at 200 V for 1 min, increasing to 3,500 V over the next 90 min followed by 65 min at 3,500 V, equilibrated (6 M urea, 30% glycerol, 50 mM Tris pH 8.8 and 2% SDS) and then run on a 10% SDS-PAGE gel containing 0.1% gelatin for the second dimension. The gel was then run and developed as previously described.

Gelatin SDS-PAGE was used to analyse the protease activity in ESWs from louse eggs both before and after hatch, and from human hair samples. An attempt was made to determine protein levels in each of the different ESW samples however this proved unsuccessful due to the very low protein levels present. Therefore, for comparative purposes, samples have been described in terms of the number of louse eggs from which the washings were obtained.

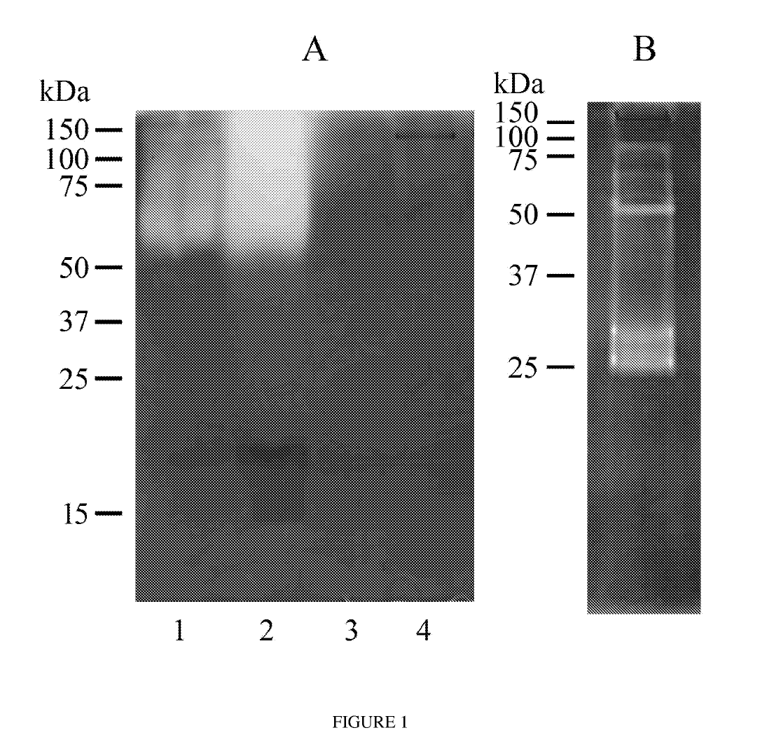

Protease activity was detected in washings from unhatched eggs within 12 hours of hatch (Sample 1) in the higher molecular weight region of the gel, above 50 kDa (FIG. 1A, Lane 1). A similar pattern of protease activity was detected in the washings taken from human hair samples following the removal of the louse eggs (Sample 2) (FIG. 1A, Lane 2). However, treatment of the hair with 1% sodium hypochlorite prior to collecting the washings (Sample 3) completely removed the protease activity (FIG. 1A, Lane 3). Hypochlorite treatment was also able to remove the extraneous proteases from unhatched louse eggs (Sample 4) (FIG. 1A, Lane 4). Hypochlorite was used to treat unhatched eggs prior to the collection of ESWs for all subsequent protease analyses.

Several distinct proteases were observed in the ESWs from hypochlorite treated eggs collected up to 2 hours post egg-hatching (Sample 5) (FIG. 1B). Bands of protease activity were detected around 25-30 kDa, 50 kDa and there were a few fainter bands detected above 50 kDa.

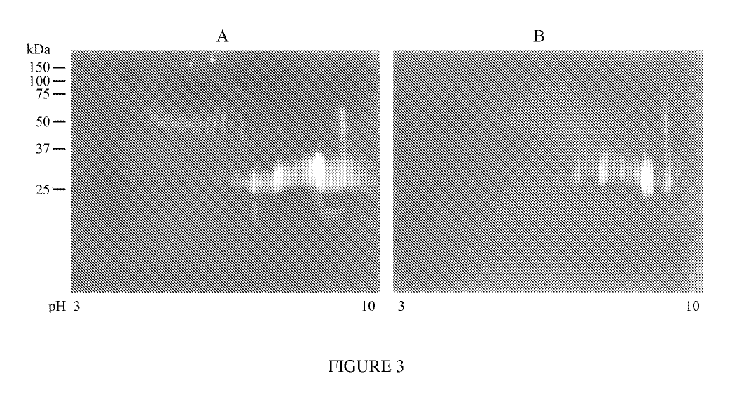

Two-dimensional gelatin SDS-PAGE was used to more accurately assess the number of protease species present in the louse ESWs. Each of the three main regions of protease activity in the one-dimensional gelatin SDS-PAGE (FIG. 1B) resolved to a number of distinct proteases when analysed by two-dimensional gelatin SDS-PAGE (FIG. 3A). The proteases present in the louse ESWs with activity in the 25-30 kDa molecular weight range resolved to at least seven distinct proteases with isoelectric points in the neutral to alkaline pH range, whereas the band of protease activity around 50 kDa resolved to at least eleven distinct protease regions with isoelectric points in the acidic to neutral pH region. The regular banding pattern of the proteases in the 50 kDa region suggests that they may be related in some manner. At least five proteases with molecular weights above 75 kDa were also observed.

In conclusion the hatching process in lice was studied by light microscopy. Egg hatching appears to be associated with physical activity of the developing nymph within the egg. However, the lack of any specialised structures for piercing or loosening the hatch flap or operculum indicates that the hatching process may also involve a biochemical component.

While highly active proteases were detected around the time of egg hatching in lice the primary source of these proteases appears to be of maternal origin. Removal of this activity prior to egg hatching was achieved using sodium hypochlorite with the lice progressing through to successfully hatch. Subsequent analysis of the ESW from freshly hatched lice indicated the presence of a number of protease species that were further investigated as targets for inhibiting egg hatching in lice.

Similar assessments can be made for other remodelling events of pest eggs.

### Example 3

### Characterisation of Proteases in Egg Shell Washings

In order to evaluate the potential of lice hatching proteases in the egg shell washings as targets for inhibiting egg hatching it was first necessary to characterize the nature of the hatching proteases. Inhibitors of the 4 major classes of proteases were used to classify the proteases in the ESW.

10% SDS-PAGE gelatine substrate gels were loaded with freeze dried egg shell washings from 100 lice eggs that had been resuspended in 50 μL of non-reducing sample buffer with samples run at 10 μL per lane. Gels were run at 4° C. for 10 minutes at 10 mA per gel followed by a further 25 minutes at 15 mA per gel. Gels were then cut into strips and each strip incubated for 2×20 minutes in a 2.5% Triton-X 100 solution containing a specific inhibitor. The inhibitors used were the serine protease inhibitor PMSF (5 mM), the metalloprotease inhibitors 1,10-phenanthroline (10 mM) and EDTA (ethylenediamine tetraacetic acid) 10 mM, the aspartic protease Pepstatin (5 μM) and the cysteine inhibitor E-64 (10 μM). The gel strips were then incubated in 0.1M Tris/HCl containing 1 mM CaCl₂ pH 8 containing the different protease inhibitors for 3 hrs at 37° C., before being stained in Coomassie blue and destained as previously described.

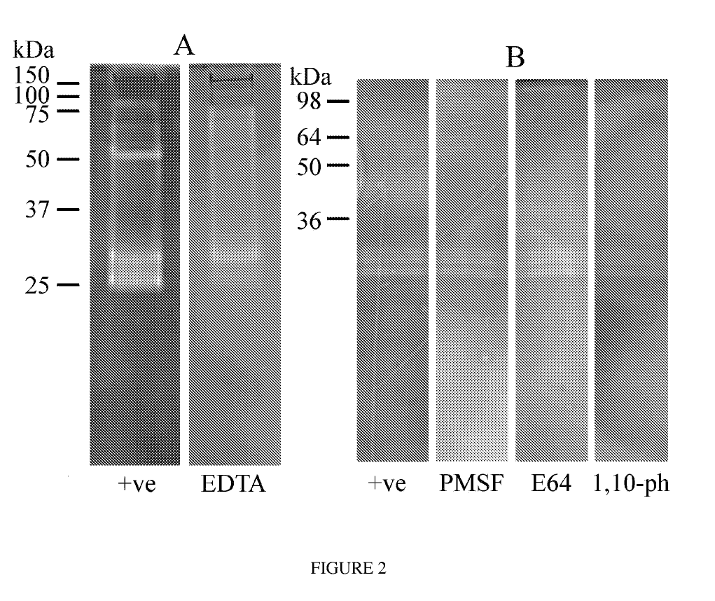

Incubation with the metal chelating agents EDTA and 1,10-phenanthroline, to inhibit metalloproteases, resulted in a reduction in protease activity compared to the untreated controls (FIGS. 2A and 2B, respectively). In contrast, there was no apparent reduction in protease activity when the ESWs were incubated with the serine/cysteine protease inhibitor PMSF (FIG. 2B), the cysteine protease inhibitor E-64 (FIG. 2B) or the aspartic protease inhibitor pepstatin (data not shown).

In order to further investigate the effect of 1,10-phenanthroline on the protease activity of the egg shell washings, the proteases were separated by two dimensional gel electrophoresis and the gel incubated in the presence of 10 mM 1,10-phenanthroline. The results from these studies confirmed the inhibitory effect of this metalloprotease inhibitor on the activity of the louse egg proteases. There was a general reduction in protease activity in the 25-30 kDa region and a clear reduction in the proteases present around the 50 kDa region and above 75 kDa (FIG. 3B).

A similar approach may be used to characterise proteases in egg shell washings of other thin membrane eggs from different pests.

### Example 4

### Development of an In Vitro Bioassay for Measuring Lice Egg Hatching

To evaluate the potential effects of protease inhibitors on lice egg hatching it was necessary to develop a reliable in vitro bioassay. Male and female clothing lice were fed on a rabbit as previously described. Female and male adult lice in a ratio of 3:1 were then transferred to a clean petri dish containing nylon cloth approximately 3×3 cm² and left for 12 hours at 32° C. During this period the female lice laid their eggs and attached them to the woven cloth. All lice would then be removed and the eggs permitted to incubate for the following 5 days. On Day 6 the cloth containing the eggs would be placed for 1 minute in a 1% sodium hypochlorite solution and then washed extensively. The eggs would then progress through to their final stages of development and hatch. In untreated control eggs a reliable average percentage hatch of between 85-95 percent was obtained using the in vitro egg hatch assay. It was subsequently found that for the egg hatching assay it was not necessary to pre-treat the lice eggs with sodium hypochlorite.

A similar approach may be used to develop in vitro bioassays for measuring other remodelling events that include, but are not limited to, thin membrane egg hatching.

### Example 5

### Identification of Compounds that can Inhibit the Activity of Lice Hatching Proteases

(a) Testing of protease inhibitors using Lice egg-hatching bioassay.

Having refined a bioassay for measuring egg hatching in lice, the next phase of the research was to use this bioassay as a means of testing the effects of different protease inhibitors on egg hatching.

Lice eggs were laid onto cloth as described above. Five days post laying the cloth containing lice eggs was removed and immersed in a 1% sodium hypochlorite solution before being washed extensively in distilled water and blotted dry on tissue paper. Lice eggs were counted under a dissecting microscope and the cloth cut into batches of between 10-30 eggs with 3-5 replicates used per treatment. The cloth containing lice eggs was then immersed in a protease inhibitor solution for a period of 10 minutes, placed on tissue paper for 1 minute to dry before being transferred to a clean petri dish and incubated until hatching. The eggs were observed at regular time intervals for evidence of eggs hatching over the next 1-2 days by which time the control eggs had hatched. Protease inhibitor solutions were typically prepared as stock solutions and added fresh at the appropriate concentration. Specifically a stock solution was prepared as follows: 1,10-phenanthroline (200 mM in methanol). In addition, the equivalent levels of the solvent were added to the non-inhibitor containing controls eggs to test for any buffer alone effects. Percentage hatch inhibition was calculated as the percentage reduction in egg hatch compared to the untreated control. The untreated control was assigned a percentage hatch of 100%.

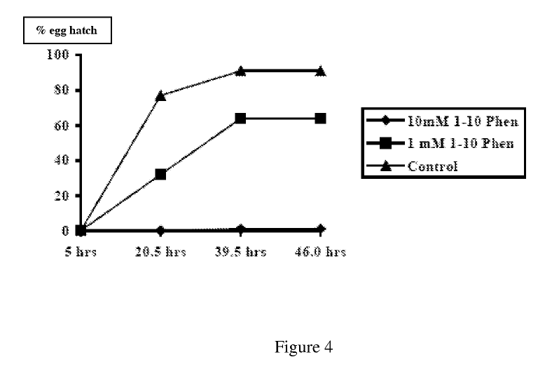

The addition of 1,10-phenanthroline, a metal chelating agent and a metalloprotease inhibitor significantly inhibited egg hatching in lice at 10 mM while at 1 mM the level of inhibition was approximately 30% compared to that of the controls (refer to FIG. 4).

These results provide data on the effect of a specific metal chelating agent and metalloprotease inhibitor on egg hatching in lice. It was however noted that when 1,10-phenanthroline was added within 24 hours of hatching, variable inhibition of egg hatching was observed (data not shown). This variability in hatch inhibition could be due to a number of factors that relate to the specific developmental stage of the louse. Furthermore these studies indicated that it is very difficult to predict the exact time of egg hatch and therefore the choice of a single time point in which to treat the eggs may be problematic when assessing the effects of a specific inhibitor on egg hatch. The in vitro assay system was therefore modified to account for this variability in lice development.

(b) Time course experiment using in the in vitro hatching assay.

A series of time-course experiments was conducted as a means of assessing inhibitors of lice egg hatching. Eggs were laid onto cloth as previously described and then at 24 hr intervals an inhibitor was added to a new group of eggs for eggs up to 120 hrs post laying. The eggs were then incubated at 28° C. for a further 8 days to permit egg hatching. This method of assaying inhibitors more closely minors the field situation where lice eggs will be at various stages of development.

The results of these studies are shown in Table 1. Significant inhibition by 1,10-phenanthroline was demonstrated at varying concentrations over the course of lice hatching. A degree of concentration dependence was also observed with the inhibitory effects of 1,10-phenanthroline. The results indicate that time-course experiments provide a more reliable means of assessing the effects of specific inhibitors on lice egg hatching.

TABLE 1
Percent inhibition of egg hatching following treatment with different
concentrations of 1,10-phenanthroline at 24 hour intervals post
egg laying.

| | Time post egg laying (hr) | | | | | |
|---|---|---|---|---|---|---|
| Inhibitor | 24 hr | 48 hr | 72 hr | 96 hr | 120 hr | 144 hr |
| 10 mM 1,10- | 100 | 100 | 100 | 100 | 100 | 100 |
| phenanthroline | | | | | | |
| 5 mM 1,10- | 100 | 100 | 100 | 100 | 93 | 96 |
| phenanthroline | | | | | | |
| 2.5 mM 1,10- | 100 | 100 | 85 | 85 | 89 | 60 |
| phenanthroline | | | | | | |

Results from the above studies indicate that lice hatching enzymes are proteases of the metallo class as judged by the ability of metal chelating agent and metalloprotease inhibitor 1,10-phenanthroline to inhibit their activity. Furthermore this compound was able to significantly inhibit egg hatching in lice at all time points examined with some evidence of a dose dependent effect particularly when eggs were treated with the lower concentrations around the time of hatching. 1,10-phenanthroline exerts its effects through its ability to chelate metal ions, preferably zinc and thereby inhibiting zinc dependent proteases.

### Example 6

### Identification of Suitable Metal Chelating Agents for Inhibiting Lice or Plutella Eggs from Hatching

A number of metal chelating agents with potential for inhibiting the remodelling events associated with thin membrane egg hatching in lice were analysed by comparing their percentage ovicidal activity in lice at different concentrations, their logit calculated pLD₅₀, Pref pLD₅₀, Activity class, clogP and molar refractivity were determined. The results are shown in Table 2.

TABLE 2

| | Lice | | | | | Activity class | | |
|---|---|---|---|---|---|---|---|---|
| | (L) | | | | | 0 = inactive | | |
| | Plutella | Ovicidal | Concentration | Logit calc | | 1 = low 2 = med | | |
| Name | (P) | activity % | mM | pLD₅₀ | Pref pLD₅₀ | 3 = high | clogP | MR |
| 1,10- | L P | 100.00% | 10 | 3.3 | >2 | 1 | 2.05 | 55 |
| Phenanthroline | | | | | | | | |
| 4,7-phenanthroline | L, P | 0.00% | 10 | 0.7 | <2 | 0 | 2.05 | 55 |
| 5,5′-dimethyl | L | 100.00% | 30 | 2.85 | >2 | 2 | 2.56 | 58 |
| bipyridine | | | | | | | | |
| 5,5′-dimethyl | L, P | 100.00% | 10 | 3.3 | | 3 | 2.56 | |
| bipyridine | | | | | | | | |
| 5,5′-dimethyl | P | 98.00% | 1 | 4.2 | 4.1 | 3 | 2.56 | 58 |
| bipyridine | | | | | | | | |
| 5,5′-dimethyl | P | 57.00% | 0.1 | 4.1 | | 3 | 2.56 | |
| bipyridine | | | | | | | | |
| 6,6′-dimethyl | L, P | 100.00% | 10 | 3.3 | >2 | 2 | 2.56 | |
| bipyridine | | | | | | | | |
| 6,6′-dimethyl | P | 100.00% | 1 | 4.3 | >3 | 2 | 2.56 | 56 |
| bipyridine | | | | | | | | |
| 6,6′-dimethyl | L P | 0.00% | 0.1 | 2.7 | <4 | 2 | 2.56 | |
| bipyridine | | | | | | | | |
| 4,4′-dimethyl | ND P | 100.00% | 10 | 3.3 | >3 | | 2.56 | |
| bipyridine | | | | | | | | |
| 4,4′-dimethyl | P | 100.00% | 1.0 | 4.3 | 4.2 | 3 | 2.56 | 58 |
| bipyridine | | | | | | | | |
| 4,4′-dimethyl | ND P | 65.00% | 0.1 | 4.2 | >3 | | 2.56 | |
| bipyridine | | | | | | | | |
| 2,2′-bipyridine | L P | 100.00% | 10 | 3.3 | >2 | 1 | 1.56 | 46 |
| 2-benzyl-pyridine | L P | 0.00% | 10 | 0.7 | <2 | 0 | 2.71 | 53 |
| 2-phenyl-pyridine | L P | 0.00% | 10 | 0.7 | <2 | 0 | 2.74 | 48 |
| 2,2′,6,2″-terpyridine | L | 0.00% | 10 | 0.7 | <2 | 0 | 2.45 | 68 |
| | ND | | | | | | | |
| 2,2′,6,2″-terpyridine | ND P | 100% | 1.0 | 4.3 | >3 | 3 | 2.45 | 68 |
| 2,2′-Bis(4,5- | L | 0.00% | 10 | 0.7 | <2 | 0 | 1.36 | 55 |
| dimethylimidazole) | ND | | | | | | | |
| 2,2′-biquinoline | L P | 0.00% | 10 | 0.7 | <2 | 0 | 4.33 | 78 |
| 2-Picoline | L | 0.00% | 10 | 0.7 | <2 | 0 | | |
| | ND | | | | | | | |
| Di(2-picolyl) amine | L | 0.00% | 10 | 0.7 | <2 | 0 | −0.33 | 59 |
| | ND | | | | | | | |
| 2,2-dipyridylamine | P | 100% | 10 | 3.3 | >2 | 1 | 1.94 | 50 |
| 2,2-dipyridylamine | ND P | 0.00% | 1.0 | 1.7 | <2 | 0 | 1.94 | 50 |
| 2-(2- | P | 100.00% | 10 | 3.3 | >2 | 1 | 2.95 | 62 |
| pyridinyl)quinoline | | | | | | | | |
| 1,3-dipyridin-3-yl- | ND P | 0.00% | 10 | 0.7 | <2 | 0 | 0.44 | 64 |
| propane-1,3-dione | | | | | | | | |
| 2,2′ Bipyridinyl-5,5′- | L | 0.00% | 10 | 0.7 | <2 | 0 | 1.74 | 70 |
| dicarboxylic acid | ND | | | | | | | |
| dimethyl ester | | | | | | | | |
| 4-Morpholin-4-yl-2- | L P | 0.00% | 10 | 0.7 | <2 | 0 | 3.36 | 86 |
| pyridin-2-yl- | | | | | | | | |
| quinoline* | | | | | | | | |
| 1,3-Bis-(4-tert-butyl- | ND P | 0.00% | 10 | 0.7 | <2 | 0 | 6.29 | 106 |
| phenyl)-propane-1,3- | | | | | | | | |
| dione | | | | | | | | |
| 1,3-Bis-(3,5- | ND, P | 0.00% | 10 | 0.7 | <2 | 0 | 4.63 | 90 |
| dimethyl-phenyl)- | | | | | | | | |
| propane-1,3-dione | | | | | | | | |
| 1,3-Bis-(4-methoxy- | ND P | 0.00% | 10 | 0.7 | <2 | 0 | 3.08 | 81 |
| phenyl)-propane-1,3- | | | | | | | | |
| dione | | | | | | | | |
| 1-(4-Chloro-phenyl)- | L | 0.00% | 10 | 0.7 | <2 | — | 3.43 | 71 |
| 3-phenyl-propane- | ND | | | | | | | |
| 1,3-dione | | | | | | | | |
| 1-(5-Chloro-2- | L P | 0.00% | 10 | 0.7 | <2 | — | 3.81 | 73 |
| hydroxy-phenyl)-3- | | | | | | | | |
| phenyl-propane-1,3- | | | | | | | | |
| dione | | | | | | | | |
| 4,4,4-Trifluoro-1- | L P | 0.00% | 10 | 0.7 | <2 | 0 | 1.65 | 47 |
| phenyl-butane-1,3- | | | | | | | | |
| dione | | | | | | | | |
| Dibenzoyl methane | P | 90.00% | 10 | 2.8 | >2 | 1 | 2.64 | 67 |
| Trans-2- | ND P | 0.00% | 10 | 0.7 | <2 | 0 | 0.11 | 32 |
| aminocyclohexanol | | | | | | | | |
| Glycine methyl ester | ND P | 0.00% | 10 | 0.7 | <2 | 0 | −0.75 | 21 |
| 2-Amino-1-phenyl | ND P | 0.00% | 10 | 0.7 | <2 | | 0.27 | 21 |
| ethanol | | | | | | | | |
| Ethyl- | ND P | 0.00% | 10 | 0.7 | <2 | 0 | 0.42 | 33 |
| acetaimidoacetate | | | | | | | | |
| Acetohydroxamic | ND P | 0.00% | 10 | 0.7 | <2 | 0 | −1.59 | 16 |
| acid | | | | | | | | |
| 2- | ND P | 0.00% | 10 | 0.7 | <2 | 0 | 0.51 | 38 |
| Acetylcyclohexanone | | | | | | | | |
| D-L-2-Amino-1- | ND P | 0.00% | 10 | 0.7 | <2 | 0 | 0.07 | 30 |
| pentanol | | | | | | | | |
| Benzohydroxaminic | ND P | 0.00% | 10 | 0.7 | <2 | 0 | 0.26 | 36 |
| acid | | | | | | | | |
| Benzoylacetone | ND P | 90.00% | 10 | 2.8 | 2.8 | 1 | 1.09 | 46 |
| Benzoylacetone* | ND P | 0.00% | 1 | 1.7 | <2 | 0 | 1.09 | 46 |
| 1-(4-tert- | L P | 0.00% | 10 | 0.7 | <2 | 0 | 4.68 | 94 |
| butylphenyl)-3-(4- | | | | | | | | |
| methoxyphenyl)- | | | | | | | | |
| propane-1,3-dione | | | | | | | | |
| 2-acetyl-1-tetralone | P | 100.00% | 10 | 3.3 | >2 | 1 | 1.53 | 55 |
| 2-acetyl-1-tetralone | P | 100.00% | 1 | 4.3 | >3 | 3 | 1.53 | 55 |

.ND refers to not done.
A blankin column 2 where either an L or a P are absent refers to no ovicidal activity observed at the concentration indicated.

### Example 7

### Effect of Washing Eggs Post Treatment with 1-10 Phenanthroline

An experiment was undertaken to determine whether washing of the eggs would effect the inhibitory activity of 1,10-phenanthroline (Table 3). A control group (5% methanol) was also set up. Percentage hatch inhibition was calculated as the percentage reduction in egg hatch compared to the untreated control. The untreated control was assigned a percentage hatch of 100%. The results from this experiment indicate that 1,10-phenanthroline is still highly efficacious at inhibiting lice egg hatching following washing of eggs in water. In later stage eggs that are approaching egg hatch (day 5) the effects appear to reflect a concentration dependence similar to that observed when lower concentrations of the inhibitor were used. It was also noted that a proportion of eggs treated with 1,10-phenanthroline had embryos that appeared to develop normally yet failed to hatch.

TABLE 3
Percent inhibition of egg hatching following treatment with 10 mM
1-10 phenanthroline at 24 hour intervals post egg laying in lice.
Lice eggs were treated with inhibitor for 10 minutes and
left unwashed or treated and washed for 1 minute and then
left to hatch.

| | Time post laying (hr) | | | | |
|---|---|---|---|---|---|
| | 24 hr | 48 hr | 72 hr | 96 hr | 120 hr |
| Treated/not | 100 | 100 | 100 | 100 | 100 |
| washed | | | | | |
| Treated/was | 100 | 100 | 100 | 97 | 62 |
| washed | | | | | |

### Example 8

### Inhibition of Hatching of Head Lice Eggs with 1-10 Phenanthroline

Tests were carried out to determine if metal chelating agent and metalloprotease inhibitor 1,10-phenanthroline could inhibit head lice egg (Pediculus humanus capitus) hatching as opposed to body lice. Head lice eggs were obtained by placing groups of both 1-2 adult male and 6-8 adult female head lice in separate wells in a 24 well petri dish containing cotton cloth. The petri dish was transferred to a humid incubator at 32° C., 70% RH for 12 hours to permit the female lice to lay their eggs. After 12 hours, all adult lice were removed from the petri dish wells and a series of time-course experiments conducted. A group of eggs (24 hr old) was treated for 10 minutes with 200 μL of a 10 mM solution of 1,10-phenanthroline. A control (ie no inhibitor treatment) group of eggs was also included. The eggs were removed from the inhibitor, blotted dry on tissue paper, placed at 32° C., 70% RH and left to hatch. A second group of eggs, (48 hours old) were treated as previously described and also left to hatch. This process was repeated at 24 hour intervals on head lice eggs up to 120 hours post laying. This method of assaying inhibitors more closely mirrors the field situation where lice eggs will be at various stages of development on the head and permits the inhibitory effects to be observed on these different stages of the parasite.

The results from the above studies indicate that 1,10-phenanthroline can significantly inhibit egg hatching in head lice (Table 4).

TABLE 4
Percent inhibition of egg hatching following treatment with
10 mM 1,10-phenanthroline at 24 hour intervals post egg
laying in lice relative to the control.

| | Days post laying | | | | |
|---|---|---|---|---|---|
| | 1 | 2 | 3 | 4 | 5 |
| Treated | 100 | 87 | 88 | 100 | 100 |

These results strongly suggest that body lice are an effective model for assaying the effects of protease inhibitors in egg hatching of head lice.

### Example 9

### Inhibition of Lice Egg Hatching with Metal Chelators

Experiments were conducted using two metal chelating agents, 2,2′-dipyridine and 6,6′-dimethyl-2,2′-dipyridine, that have clogP values of 1.56 and 2.56 respectively and molar refractivities of 46 cm³/mol and 56 cm³/mol respectively, to determine their effects on lice egg hatching. These compounds were tested in the standard lice assay to determine their ovicidal effects (refer to example 5 for method used to test inhibitors). The results of this study are shown in Tables 5 and 6.

TABLE 5
Results of egg hatching following treatment with 2,2′-dipyridyl at 24 hour
intervals post egg laying.

| | 24 hr | | | 48 hr | | | 72 hr | | | 96 hr | | | 120 hr | | |
|---|---|---|---|---|---|---|---|---|---|---|---|---|---|---|---|
| Replicates | N | H | Ph | N | H | Ph | N | H | Ph | N | H | Ph | N | H | Ph |
| 1 | 7 | 0 | 0 | 6 | 0 | 0 | 7 | 0 | 0 | 13 | 0 | 0 | 10 | 0 | 0 |
| 2 | 8 | 0 | 0 | 14 | 0 | 0 | 7 | 0 | 0 | 9 | 1 | 0 | 10 | 0 | 0 |
| 3 | 11 | 0 | 0 | — | — | — | 14 | 0 | 0 | 10 | 0 | 0 | 13 | 0 | 0 |

The results are indicated for:
N (number of eggs per replicate),
H (number of eggs successfully hatched) and
Ph (number of eggs partly hatched).

TABLE 6
Results of egg hatching following treatment with 6,6′-Dimethyl-2,2′-dipyridyl
at 24 hour intervals post egg laying.

| | 24 hr | | | 48 hr | | | 72 hr | | | 96 hr | | | 120 hr | | |
|---|---|---|---|---|---|---|---|---|---|---|---|---|---|---|---|
| Replicates | N | H | Ph | N | H | Ph | N | H | Ph | N | H | Ph | N | H | Ph |
| 1 | 10 | | 0 | 13 | 0 | 0 | 15 | 0 | 0 | 25 | 0 | 0 | 23 | 0 | 0 |
| 2 | 10 | 0 | 0 | 11 | 0 | 0 | 16 | 0 | 0 | 22 | 0 | 0 | 9 | 0 | 0 |
| 3 | 11 | 0 | 0 | 6 | 0 | 0 | 10 | 0 | 0 | 18 | 0 | 0 | — | — | — |

The results are indicated for:
N (number of eggs per replicate),
(number of eggs successfully hatched) and
Ph (number of eggs partly hatched).

The results from these studies indicate that both 6,6′-Dimethyl-2,2′-dipyridyl and 2,2′-dipyridyl displayed very strong ovicidal activity whereby lice egg hatching was completely inhibited at all time points examined. Both 6,6′-Dimethyl-2,2′-dipyridyl and 2,2′-dipyridyl are metal chelating agents and metalloprotease inhibitors that are non-intercalating.

### Example 10

### Comparative Assessment of Commercial Lice Products with 1,10-Phenanthroline

The ovicidal properties of three major commercial head lice products were evaluated in the standard lice egg-hatching assay. The 3 commercial head lice products were as follows:

1. KP-24® Nelson Laboratories, active ingredients 1% maldison (malathion);

2. RID® Bayer, active ingredients, 1% pyrethrins; and

3. NIX® Pfizer, active ingredients, 1% permethrin.

These three products were tested according to manufacturer's recommendations. Groups of eggs (24 hours old) were treated with the different products according to manufacturer's recommendations for the appropriate period of time (5-10 minutes) followed by a rinse for 1-2 minutes in 32° C. water. A positive controls (10 mM 1,10-phenanthroline) and two negative controls (no treatment and 20% Methanol) were also incorporated. Post exposure to the different products, the eggs were rinsed with warm water at 32° C. before being blotted dry on tissue paper and placed at 32° C., 70% RH and left to hatch. A second group of eggs, (48 hours old) were treated as previously described and also left to hatch. This process was repeated at 24 hour intervals on head lice eggs up to 120 hours post laying. This method of assaying inhibitors more closely mirrors the field situation where lice eggs will be at various stages of development on the head and permits the inhibitory effects to be observed on these different stages of the parasite. The results of these studies are shown in Table 7.

TABLE 7
Results of egg hatching following treatment with 3 commercial head lice
products, 10 mM 1,10-phenanthroline and controls at 24 hour intervals
post egg laying.

| | 24 hr | | | 48 hr | | | 72 hr | | | 96 hr | | | 120 hr | | |
|---|---|---|---|---|---|---|---|---|---|---|---|---|---|---|---|
| Replicates | N | H | Ph | N | H | Ph | N | H | Ph | N | H | Ph | N | H | Ph |

NIX-Pfizer

| | | | | | | | | | | | | | | | |
|---|---|---|---|---|---|---|---|---|---|---|---|---|---|---|---|
| 1 | 16 | 12 | 2 | 9 | 7 | 0 | 18 | 3 | 3 | 12 | 8 | 3 | 19 | 12 | 3 |
| 2 | 10 | 4 | 3 | 6 | 2 | 3 | 10 | 3 | 3 | 15 | 7 | 5 | 18 | 8 | 7 |
| 3 | 10 | 7 | 2 | 9 | 4 | 3 | 17 | 5 | 7 | — | — | — | 36 | 21 | 5 |

RID-Bayer

| | | | | | | | | | | | | | | | |
|---|---|---|---|---|---|---|---|---|---|---|---|---|---|---|---|
| 1 | 8 | 0 | 3 | 12 | 3 | 4 | 7 | 0 | 0 | 8 | 0 | 0 | 14 | 0 | 1 |
| 2 | 8 | 2 | 5 | 7 | 0 | 1 | 5 | 1 | 2 | 8 | 0 | 0 | — | — | — |
| 3 | 5 | 0 | 2 | 10 | 0 | 2 | 6 | 1 | 3 | 11 | 0 | 0 | — | — | — |

KP24KP24

| | | | | | | | | | | | | | | | |
|---|---|---|---|---|---|---|---|---|---|---|---|---|---|---|---|
| 1 | 7 | 7 | 0 | 10 | 10 | 0 | 10 | 1 | 3 | 10 | 0 | 0 | 10 | 0 | 0 |
| 2 | 6 | 6 | 0 | 10 | 9 | 0 | 0 | 0 | 0 | 7 | 0 | 0 | 8 | 0 | 0 |
| 3 | 9 | 8 | 0 | — | — | — | — | — | — | — | — | — | 12 | 0 | 1 |

1,10-phenanthroline (10 mM)

| 1 | 13 | 0 | 0 | 5 | 0 | 0 | 7 | 0 | 0 | 10 | 0 | 0 | 9 | 0 | 0 |
|---|---|---|---|---|---|---|---|---|---|---|---|---|---|---|---|
| 2 | 9 | 0 | 0 | 15 | 0 | 0 | 7 | 0 | 0 | 10 | 0 | 0 | 6 | 4 | 0 |
| 3 | — | — | — | 8 | 0 | 0 | 9 | 0 | 0 | — | — | — | 7 | 1 | 0 |
| | 24 hr | | | 48 hr | | | 72 hr | | | 96 hr | | | 120 hr | | |
| Replicates | N | H | Ph | N | H | P | N | H | P | N | H | P | N | H | Ph |

Control (20% Methanol)

| | | | | | | | | | | | | | | | |
|---|---|---|---|---|---|---|---|---|---|---|---|---|---|---|---|
| 1 | — | — | — | 14 | 14 | 0 | 10 | 10 | 0 | 10 | 10 | 0 | 13 | 13 | 0 |
| 2 | — | — | — | 5 | 4 | 0 | 8 | 8 | 0 | 10 | 9 | 0 | 7 | 7 | 0 |
| 3 | — | — | — | — | — | 0 | 9 | 7 | 0 | 4 | 4 | 0 | 10 | 10 | 0 |

Control (Untreated)

| | | | | | | | | | | | | | | | |
|---|---|---|---|---|---|---|---|---|---|---|---|---|---|---|---|
| 1 | 10 | 9 | 0 | 11 | 11 | 0 | 25 | 24 | 0 | 10 | 8 | 0 | 20 | 20 | 0 |
| 2 | 20 | 18 | 0 | 8 | 7 | 0 | 10 | 10 | 0 | 11 | 10 | 0 | 20 | 18 | 0 |
| 3 | — | — | — | 8 | 8 | 0 | — | — | — | 10 | 10 | 0 | — | — | — |

The results are indicated for:
N (number of eggs per replicate),
H (number of eggs successfully hatched) and
Ph (number of eggs partly hatched).

Results from the testing of 3 commercial pediculicides indicate that they displayed inconsistent levels of ovicidal activity across the different stages of lice egg hatching. Whereas the compound 1,10-phenanthroline was highly effective at inhibiting lice egg hatching.

### Example 11

### Assessment of Additional Commercial Lice Products

The ovicidal properties of two major commercial head lice products were evaluated in the standard lice egg-hatching assay. The 2 commercial head lice products were as follows:

1. Pronto Plus® Shampoo Del Laboratories, active ingredients 0.33% Pyrethrins; and

2. Pronto Plus® Mousse Shampoo Del Laboratories, active ingredients, 0.33% Pyrethrins.

These two products were tested according to manufacturer's recommendations. Groups of eggs (24 hours old) were treated with the different products according to manufacturer's recommendations for the appropriate period of time (5-10 minutes) followed by a rinse for 1-2 minutes in 32° C. water. Two negative controls (no treatment and 20% ethanol) were also incorporated. Post exposure to the different products, the eggs were blotted dry on tissue paper and placed at 32° C., 70% RH and left to hatch. A second group of eggs, (48 hours old) were treated as previously described and also left to hatch. This process was repeated at 24 hour intervals on head lice eggs up to 120 hours post laying. This method of assaying inhibitors more closely mirrors the field situation where lice eggs will be at various stages of development on the head and permits the inhibitory effects to be observed on these different stages of the parasite. The results of these studies are shown in Table 8.

TABLE 8
Results of egg hatching following treatment with 2 commercial head lice
products and controls at 24 hour intervals post egg laying.
Pronto Plus Shampoo

| | 24 hr | | | 48 hr | | | 72 hr | | | 96 hr | | | 120 hr | | |
|---|---|---|---|---|---|---|---|---|---|---|---|---|---|---|---|
| Replicates | N | H | Ph | N | H | P | N | H | P | N | H | P | N | H | Ph |
| 1 | 14 | 10 | 2 | 11 | 9 | 0 | 30 | 27 | 0 | 35 | 30 | 0 | 40 | 38 | 2 |
| 2 | 20 | 15 | 3 | 21 | 18 | 0 | 19 | 16 | 0 | 42 | 36 | 0 | 38 | 29 | 5 |
| 3 | — | — | — | — | — | — | — | — | — | — | — | — | — | — | — |

Pronto Plus Mousse Shampoo

| | 24 hr | | | 48 hr | | | 72 hr | | | 96 hr | | | 120 hr | | |
|---|---|---|---|---|---|---|---|---|---|---|---|---|---|---|---|
| Replicates | N | H | Ph | N | H | P | N | H | Ph | N | H | Ph | N | H | Ph |
| 1 | 10 | 8 | 0 | 18 | 15 | 0 | 47 | 31 | 9 | 63 | 8 | 34 | 51 | 7 | 40 |
| 2 | 15 | 13 | 0 | 10 | 6 | 0 | 30 | 14 | 8 | 29 | 5 | 10 | 50 | 8 | 30 |
| 3 | 11 | 9 | 0 | — | — | — | 34 | 13 | 17 | 21 | 1 | 15 | 31 | 1 | 17 |
| | 24 hr | | | 48 hr | | | 72 hr | | | 96 hr | | | 120 hr | | |
| Replicates | N | H | Ph | N | H | P | N | H | P | N | H | P | N | H | P |

Control (ethanol)

| | | | | | | | | | | | | | | | |
|---|---|---|---|---|---|---|---|---|---|---|---|---|---|---|---|
| 1 | 12 | 10 | 0 | 18 | 16 | 0 | 40 | 36 | 1 | 21 | 20 | 0 | 49 | 47 | 0 |
| 2 | 11 | 9 | 0 | 21 | 18 | 0 | 41 | 37 | 0 | 28 | 26 | 0 | 39 | 36 | 0 |
| 3 | 11 | 11 | 0 | 13 | 11 | 0 | 75 | 70 | 0 | 29 | 27 | 0 | 36 | 34 | 0 |

Control (untreated)

| | | | | | | | | | | | | | | | |
|---|---|---|---|---|---|---|---|---|---|---|---|---|---|---|---|
| 1 | 10 | 9 | 0 | 27 | 26 | 0 | 61 | 60 | 0 | 50 | 49 | 1 | 48 | 46 | 0 |
| 2 | — | — | — | — | — | — | — | — | — | — | — | — | — | — | — |
| 3 | — | — | — | — | — | — | — | — | — | — | — | — | — | — | — |

The results are indicated for:
N (number of eggs per replicate),
H (number of eggs successfully hatched) and
Ph (number of eggs partly hatched).

Results from the testing of 2 commercial pediculicides indicate that they displayed very poor and inconsistent ovicidal activity across the different stages of lice egg hatching.

### Example 12

### Evaluation of Compounds on Egg Hatching of Plutella xylostella

Several hundred Plutella xylostella eggs (Waite strain) were collected, that had been laid over a 24 hour period. Within 3-5 hours of collection, the eggs were treated with different inhibitors as described below.

Batches of Plutella eggs that had been laid on either fine cloth or parafilm were dipped in a specific inhibitor solution for between 2-10 seconds, the excess solution was drained by blotting with dry tissue paper. The egg masses were then placed in a humid box at 25° C. until egg hatch. Control eggs were exposed to absolute methanol as described above. At day 6 post laying the eggs were assessed from the different treatments and the percentage of egg hatch determined relative to the control as shown in Table 9.

TABLE 9
Ovicidal effects of inhibitors on egg hatch of Plutella xylostella
relative to control.

| | Number | Number | % |
|---|---|---|---|
| Inhibitor | hatched | unhatched | Inhibition |
| 6,6′-dimethyl-2,2′-dipyridyl (10 mM) | 0 | 79 | 100 |
| 6,6′-dimethyl-2,2′-dipyridyl (1 mM) | 0 | 26 | 100 |
| 6,6′-dimethyl-2,2′-dipyridyl (0.1 mM) | 23 | 29 | 0 |
| 6,6′-dimethyl-2,2′-dipyridyl (0.01 mM) | 13 | 7 | 0 |
| 6,6′-dimethyl-2,2′-dipyridyl (0.001 mM) | 11 | 6 | 0 |
| 1,10-phenanthroline (10 mM) | 0 | 45 | 100 |
| 1,10-phenanthroline (1 mM) | 15 | 16 | 0 |
| Control (100% MeOH) | 63 | 92 | — |

Table 9 indicates that the metal chelator 6,6′ dimethyl-2,2′ dipyridyl was able to inhibit egg hatching in Plutella xylostella in a dose dependent manner, with strong ovicidal effects evident at both 10 and 1 mM. In addition, the metalloprotease inhibitor/metal chelator, 1,10-phenanthroline was also able to significantly inhibit egg hatching of this insect at 10 mM.

### Example 13

### Evaluation of Compounds on Egg Hatching of Plutella xylostella

Several hundred Plutella xylostella eggs (Waite strain) were collected, that had been laid over a 24 hour period. Within 3-5 hours of collection, all of the eggs were treated with different inhibitors as described below.

Batches of Plutella eggs that were laid on either fine cloth or parafilm were dipped in a specific inhibitor solution for between 2-10 seconds, the excess solution was drained by blotting with dry tissue paper. The egg masses were then placed in a humid box at 25 degrees until egg hatch. Control eggs were exposed to absolute methanol as described above or not treated. At day 6 post laying the eggs were assessed from the different treatments and the percentage of egg hatch determined relative to the controls as shown in Tables 10 and 11.

TABLE 10
Ovicidal effects of inhibitors on egg hatch of Plutella xylostella
relative to controls (eggs laid on cloth).

| | Number | Number | % |
|---|---|---|---|
| Inhibitor | hatched | unhatched | Inhibition |
| 6,6′-dimethyl-2,2′-dipyridyl (10 mM) | 0 | 53 | 100 |
| 6,6′-dimethyl-2,2′-dipyridyl (1 mM) | 0 | 23 | 100 |
| 6,6′-dimethyl-2,2′-dipyridyl (0.1 mM) | 49 | 0 | 0 |
| 6,6′-dimethyl-2,2′-dipyridyl (0.01 mM) | 23 | 4 | 12 |
| 5,5′-dimethyl-2,2′-dipyridyl (10 mM) | 0 | 21 | 100 |
| 5,5′-dimethyl-2,2′-dipyridyl (1 mM) | 5 | 22 | 78 |
| 4,4′-dimethyl-2,2′-dipyridyl (10 mM) | 0 | 36 | 100 |
| 4,4′-dimethyl-2,2′-dipyridyl (1 mM) | 0 | 30 | 100 |
| Control (untreated) | 32 | 1 | — |
| Control (100% MeOH) | 34 | 1 | — |

TABLE 11
Ovicidal effects of inhibitors on egg hatch of Plutella xylostella
relative to controls (eggs laid on parafilm).

| | Number | Number | % |
|---|---|---|---|
| Inhibitor | hatched | unhatched | Inhibition |
| 6,6′-dimethyl-2,2′-dipyridyl (10 mM) | 0 | 106 | 100 |
| 6,6′-dimethyl-2,2′-dipyridyl (1 mM) | 0 | 63 | 100 |
| 6,6′-dimethyl-2,2′-dipyridyl (0.1 mM) | 65 | 11 | 7 |
| 6,6′-dimethyl-2,2′-dipyridyl (0.01 mM) | 92 | 1 | 0 |
| 5,5′-dimethyl-2,2′-dipyridyl (10 mM) | 0 | 138 | 100 |
| 5,5′-dimethyl-2,2′-dipyridyl (1 mM) | 18 | 133 | 88 |
| 4,4′-dimethyl-2,2′-dipyridyl (10 mM) | 0 | 139 | 100 |
| 4,4′-dimethyl-2,2′-dipyridyl(1 mM) | 10 | 107 | 91 |
| Control (untreated) | 108 | 3 | — |
| Control (100% MeOH) | 58 | 7 | — |

Tables 10 and 11 show the effects of exposing Plutella xylostella eggs to selected dipyridyl compounds on egg hatching relative to controls. The results show a dose dependent effect for 6,6′-dimethyl-2,2′ dipyridyl with both 10 and 1 mM being effective at inhibiting egg hatching of the Plutella eggs. At 0.1 and 0.01 mM, there was no observable effects on egg hatching. These results confirm the results shown in Example 12 for this compound. In addition, both 5,5′-dimethyl-2,2′ dipyridyl and 4,4′-dimethyl-2,2′ dipyridyl were able to significantly inhibit egg hatching at both 10 and 1 mM.

There were no significant differences observed between eggs laid on either cloth or parafilm.

### Example 14

### Control of Helicoverpa spp. with Industry Standard

Lannate® (Crop Care Australasia Pty Ltd) containing methomyl as an active compound was chosen as a comparative control.

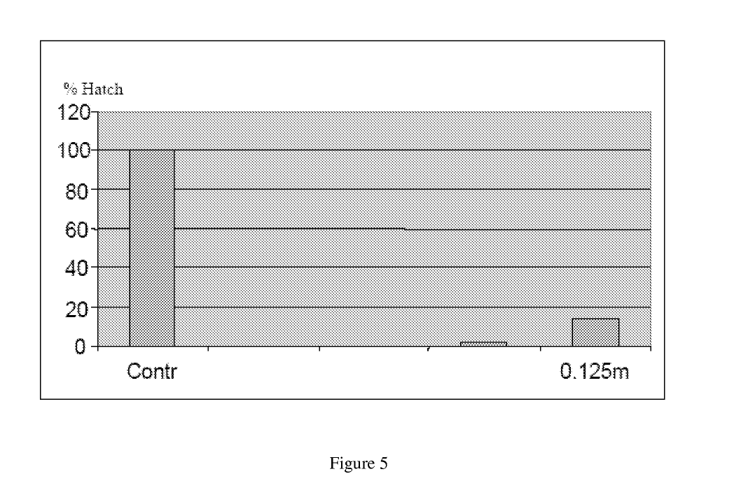

For the control of Helicoverpa on cotton in the field an application rate of 200 mL/100 L is recommended. This equates to approximately 2.5 mM of the active compound. A range of concentrations were made up in water and the eggs placed in the solutions for approximately 2-10 seconds. The eggs which had been laid on cloth were then blotted dry and placed in an incubator at 26° C. for 3-4 days until hatch. Hatch rates were then assessed compared to a water only treated control (FIG. 5). The results indicate a strong dose titration effect of Lannate® against Helicoverpa eggs with very good efficacy evident at 1.25 mM.

### Example 15

### Control of Helicoverpa spp with 2-acetyl-1-tetralone

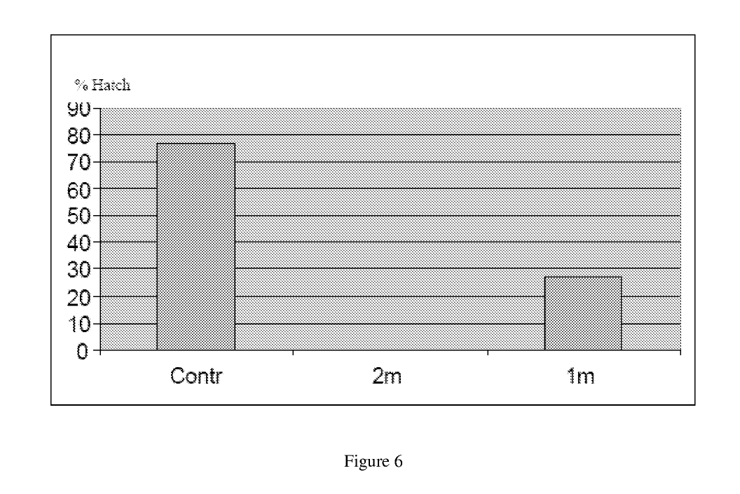

The ovicidal activity of 2-acetyl-1-tetralone against H. armigera eggs was assessed as described in Example 14. The compound was dissolved in 100% diethoxyglycol and diluted to a final concentration of 1% in water. The results are given in FIG. 6 and show that 2-acetyl-1-tetralone displayed strong ovicidal efficacy at 2 mM with efficacy declining at 1 mM.

### Example 16

### Evaluation of Compounds on Egg Hatching of Helicoverpa armigera

Several hundred Helicoverpa armigera eggs (Tatura×Toowoomba strains) were collected, that had been laid on fine mesh cloth over a 24 hour period. Within 3-5 hours of collection, all of the eggs were treated with different inhibitors as described below.

Batches of Helicoverpa eggs were exposed to a specific inhibitor solution for between 2-10 seconds the excess solution drained by blotting with dry tissue paper. The egg masses were then placed in a humid box at 25 degrees until egg hatch. Control eggs were exposed to absolute methanol as described above. At day 6 post laying the eggs were assessed from the different treatments and the percentage of egg hatch determined relative to the control as shown in Table 12.

TABLE 12
Ovicidal effects of inhibitors on egg hatch of Helicoverpa armigera
eggs relative to control.

| | Number | Number | % |
|---|---|---|---|
| Inhibitor | hatched | unhatched | Inhibition |
| 6,6′-dimethyl-2,2′-dipyridyl (10 mM) | 7 | 98 | 94 |
| 6,6′-dimethyl-2,2′-dipyridyl (1 mM) | 4 | 140 | 97 |
| 6,6′-dimethyl-2,2′-dipyridyl (0.1 mM) | na | na | 0 |
| 6,6′-dimethyl-2,2′-dipyridyl (0.01 mM) | na | na | 0 |
| 6,6′-dimethyl-2,2′-dipyridyl (0.001 mM) | na | na | 0 |
| 1,10-phenantholine (10 mM) | 31 | 16 | 44 |
| 1,10-phenanthroline (1 mM) | na | na | 0 |
| Control (100% MeOH) | na | na | 0 |

na refers to all of the eggs hatching and being devoured by the newly hatched caterpillars.

The results in Table 12 indicate that 6,6′ dimethyl-2,2′ dipyridyl was able to significantly inhibit egg hatching of Helcoverpa armigera eggs at 10 and 1 mM. No inhibition was recorded at concentrations below this level. The compound 1,10-phenanthroline was also able to inhibit egg hatching at 10 mM only.

### Example 17

### Evaluation of Compounds on Egg Hatching of Helicoverpa armigera

Several hundred Helicoverpa armigera eggs (Tatura×Toowoomba strains) were collected, that had been laid on fine mesh cloth over a 24 hour period. Within 3-5 hours of collection, all of the eggs were treated with different inhibitors as described below.

Batches of Helicoverpa eggs were then exposed to a specific inhibitor solution for between 2-10 seconds the excess solution drained by blotting with dry tissue paper. The egg masses were then placed in a humid box at 25° C. until egg hatch. Control eggs were exposed to absolute methanol as described above. At day 6 post laying the eggs were assessed from the different treatments and the percentage of egg hatch determined relative to the control as shown in Table 13.

TABLE 13
Ovicidal effects of inhibitors on egg hatch of Helicoverpa armigera
relative to the control.

| | Number | Number | |
|---|---|---|---|
| Inhibitor | hatched | unhatched | % Inhibition |
| 6,6′-dimethyl-2,2′-dipyridyl (10 mM) | 3 | 48 | 94 |
| 6,6′-dimethyl-2,2′-dipyridyl (1 mM) | 2 | 61 | 97 |
| 6,6′-dimethyl-2,2′-dipyridyl (0.1 mM) | 70 | 0 | 0 |
| 5,5′-dimethyl-2,2′-dipyridyl (10 mM) | 0 | 42 | 100 |
| 4,4′-dimethyl-2,2′-dipyridyl (10 mM) | 8 | 43 | 84 |
| 4,4′-dimethyl-2,2′-dipyridyl(1 mM) | 23 | 29 | 66 |
| Control (100% MeOH) | 37 | 2 | — |

The data presented in Table 13, support the previous results provided in Example 4 demonstrating that 6,6′-dimethyl-2,2′-dipyridyl is able to significantly inhibit the egg hatching of Helicoverpa armigera eggs at both 10 and 1 mM. At 0.1 mM, no inhibition of egg hatching was observed with this compound. In addition, data is presented that indicates significant inhibition of egg hatching at 10 mM for both 5,5′-dimethyl-2,2′-dipyridyl and 4,4′-dimethyl-2,2′-dipyridyl. In addition, significant inhibition of egg hatching was observed at 1 mM 4,4′-dimethyl-2,2′-dipyridyl.

### Example 18

### Evaluation of Effects of 2-(2-pyridinyl)quinone on Hatching of Plutella xylostella Eggs

Several hundred Plutella xylostella eggs (Waite strain) were collected, that had been laid over a 24 hour period. Within 24-48 hours of collection, the eggs were treated with different inhibitors as described below.

Batches of Plutella eggs that had been laid on fine cloth were dipped in a specific inhibitor solution for approximately 2 seconds, the excess solution was drained by blotting with dry tissue paper. The egg masses were then placed in a humid box at 25 degrees until egg hatch. Control eggs were exposed to absolute ethanol as described above. On day 6 post laying the eggs were assessed from the different treatments and the percentage of egg hatch determined relative to the control.

TABLE 14
Ovicidal effects of inhibitors on egg hatch of Plutella xylostella
relative to control.

| | Number | Number | |
|---|---|---|---|
| Inhibitor | hatched | unhatched | % Inhibition |
| 2-(2-pyridinyl)quinoline (10 mM) | 2 | 56 | 96 |
| Control (100% ETOH) | 55 | 14 | — |

Table 14 indicates that the metal chelating compound 2-(2-pyridinyl)quinoline was able to inhibit egg hatching in Plutella xylostella at 10 mM.

### Example 19

### Evaluation of Effects of Added Metal Ions on Inhibition of Egg Hatching by 6,6′-dimethyl-2-29-dypyridyl

Several hundred Plutella xylostella eggs (Waite strain) were collected, that had been laid over a 24 hour period. Within 24 hours of collection the following experimental design was chosen. Batches of eggs were exposed to 10 mM 6,6′-dimethyl-2,2′-dipyridyl for 2 seconds while additional batches of eggs were exposed to the solvent only (Methanol) for 2 seconds. All batches of eggs were allowed to air dry for 20 minutes at room temperature. The eggs were then given a 2 second exposure to FeSO₄ at either 10, 5 or 1 mM, air dried and put in the incubator at 24° C. and allowed to hatch over the next 6 days. In addition, a positive control of 10 mM 6,6′-dimethyl-2,2′-dipyridyl was set up in which eggs were exposed to this compound for 2 seconds, air dried and placed in the incubator.

TABLE 15
Reversal of the ovicidal effects of 10 mM 6,6′-dimethyl-2,2′-dipyridyl
on egg hatch of Plutella xylostella relative to the FeSO₄ controls.

| | Number | Number | % |
|---|---|---|---|
| Inhibitor | hatched | unhatched | Inhibition |
| 6,6′-dimethyl-2,2′-dipyridyl (+ve) | 0 | 44 | 100 |
| control | | | |
| 6,6′-dimethyl-2,2′-dipyridyl followed | 3 | 28 | 90 |
| by 2 second exposure to MEOH | | | |
| 6,6′-dimethyl-2,2′-dipyridyl followed | 12 | 19 | 38 |
| by 2 second exposure to 10 mM FeSO₄ | | | |
| 6,6′-dimethyl-2,2′-dipyridyl followed | 25 | 0 | 0 |
| by 2 second exposure to 5 mM FeSO₄ | | | |
| 6,6′-dimethyl-2,2′-dipyridyl followed | 33 | 1 | 3 |
| by 2 second exposure to 1 mM FeSO₄ | | | |

Results presented in Table 15 indicate that the addition of the divalent metal ions in the form of Fe in FeSO₄ was able to reverse the effects of the metal chelating agent 6,6′-dimethyl-2,2′-dipyridyl. The results indicate that the reversal of the inhibitory effects of 6,6′-dimethyl-2,2′-dipyridyl are due to Fe replacing the action of this inhibitor as opposed to a simple dilution of the inhibitor by the FeSO₄. This effect is indicated by the finding that exposure of the eggs to MeOH alone post exposure to the inhibitor still resulted in a significant degree of inhibition of egg hatching.

### Example 20

### Evaluation of Effects of Added Metal Ions on Inhibition of Egg Hatching by 5,5′-dimethyl-2,2′-dipyridyl

Several hundred Plutella xylostella eggs (Waite strain) were collected, that had been laid over a 24 hour period. Within 24 hours of collection the following experimental design was chosen. Batches of eggs were exposed to 10 mM 5,5′-dimethyl-2,2′-dipyridyl for 2 seconds while additional batches of eggs were exposed to the solvent only (Methanol) for 2 seconds. All batches of eggs were allowed to air dry for 20 minutes at room temperature. The eggs were then given a 2 second exposure to FeSO₄ at 10, 5 or 1 mM, air dried and put in an incubator at 24° C. and allowed to hatch over the next 6 days. In addition, a positive control of 10 mM, 5,5′-dimethyl-2,2′-dipyridyl was set up in which eggs were exposed to this compound for 2 seconds, air dried and placed in the incubator.

TABLE 16
Reversal of the ovicidal effects of 10 mM 5,5′-dimethyl-2,2′-dipyridyl
on egg hatch of Plutella xylostella relative to the FeSO₄ only controls.

| | Number | Number | % |
|---|---|---|---|
| Inhibitor | hatched | unhatched | Inhibition |
| 5,5′-dimethyl-2,2′-dipyridyl (+ve) | 0 | 38 | 100 |
| control | | | |
| 5,5′-dimethyl-2,2′-dipyridyl followed | 16 | 19 | 55 |
| by 2 second exposure to MEOH | | | |
| 5,5′-dimethyl-2,2′-dipyridyl followed | 23 | 2 | 8 |
| by 2 second exposure to 10 mM FeSO₄ | | | |
| 5,5′-dimethyl-2,2′-dipyridyl followed | 25 | 0 | 0 |
| by 2 second exposure to 5 mM FeSO₄ | | | |
| 5,5′-dimethyl-2,2′-dipyridyl followed | 39 | 0 | 0 |
| by 2 second exposure to 1 mM FeSO₄ | | | |

Results presented in Table 16 indicate that the addition of the divalent metal ions in the for of Fe in FeSO₄ was able to reverse the effects of the metal chelating agent 5,5′-dimethyl-2,2′-dipyridyl. The results indicate that the reversal of the inhibitory effects of 5,5′-dimethyl-2,2′-dipyridyl are due to Fe removing the action of this inhibitor as opposed to a simple dilution of the inhibitor by the FeSO₄. This effect is indicated by the finding that exposure of the eggs to MeOH alone post exposure to the inhibitor still resulted in a significant degree of inhibition of egg hatching.

### Example 21

### Ovicidal Efficacy of Metal Chelating Compounds Against Plutella

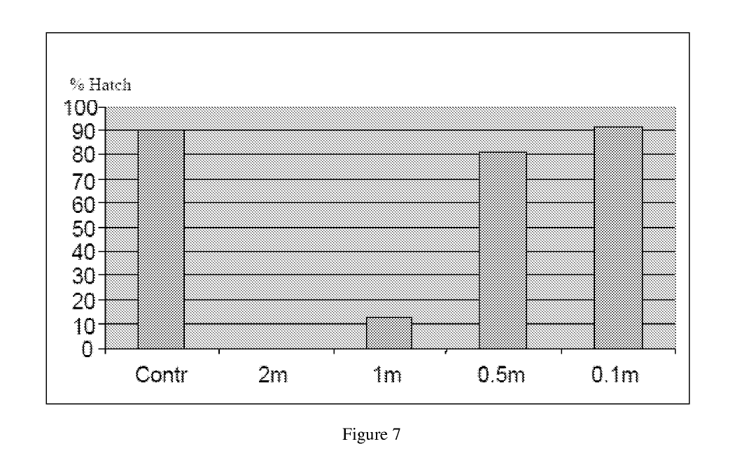

The ovicidal efficacy of 2-acetyl-1-tetralone was also tested against Plutella eggs. The compound was dissolved in diethoxyglycol and then diluted to a final concentration of 1% diethoxyglycol containing 1 mM 2-acetyl-1-tetralone and tested in the same manner as in Example 13. The results of this assay are given in FIG. 7 and indicate strong ovicidal efficacy of this compound against Plutella at 2 and 1 mM with no inhibition observed at 0.5 mM.

### Example 22

### Evaluation of Compounds on Egg Hatching of Plutella xylostella

The ovicidal efficacy of 2,2′,6,2″-terpyridine and 5,5′-diethyl-2,2′ dipyridyl was also tested against Plutella eggs. The compounds were dissolved in diethoxyglycol and then diluted to a final concentration of 1% diethoxyglycol containing 1 mM 2,2′,6,2″-terpyridine or 1 mM and 0.1 mM 5,5′-diethyl-2,2′-dipyridyl and tested in the same manner as in Example 13. The results of this assay are given in Table 17 and indicate complete inhibition at 1 mM for both compounds. Partial inhibition of egg hatching was observed at 0.1 mM 5,5′-diethyl-2,2′-dipyridyl.

TABLE 17
Ovicidal effects of compounds on egg hatch of Plutella xylostella
relative to controls (eggs laid on cloth).

| | Number | Number | |
|---|---|---|---|
| Inhibitor | hatched | unhatched | % Inhibition |
| 5,5′-diethyl-2,2′-dipyridyl (1 mM) | 0 | 34 | 100 |
| 5,5′-diethyl-2,2′-dipyridyl (0.1 mM) | 22 | 34 | 31 |
| 2,2′,6,2″-terpyridine (1 mM) | 0 | 46 | 100 |
| Control (1% diethoxyglycol) | 37 | 39 | — |

### Example 23

### Effects of 6,6′-dimethyl-2,2′-dipyridyl and 5,5′-dimethyl-2,2′-dipyridyl on Egg Hatching in Bovicola ovis

B. ovis eggs were collected from the wool of sheep that were infested with this parasite. The eggs were collected using forceps and with the aid of a dissecting microscope and placed in 24 well tissue culture plates in duplicate lots of 10 eggs per replicate. The eggs were then exposed to either methanol alone (control) or the test compounds for either 10 minutes or 1 minute before being removed from the wells and placed into individual glass vials containing a diet at the base of the tube. The tubes were placed in plastic containers containing a salt solution (to keep humidity constant at 68%) and the containers maintained at a temperature 32° C. The eggs were monitored for hatching over the following 12 days and % hatch inhibition determined in comparison to the controls.

TABLE 18
Effects of 6,6′-dimethyl-2,2′-dipyridyl and 5,5′-dimethyl-2,2′-dipyridyl
on egg hatching in Bovicola ovis.

| | Number | | |
|---|---|---|---|
| | hatched in | | |
| | different | Number | |
| Inhibitor | replicates | unhatched | % Inhibition |
| 10 mM 5,5′-dimethyl-2,2′-dipyridyl | Rep 1. 0 | 10 | 100 |
| (10 minute exposure) | Rep 2. 0 | 10 | |
| 10 mM 5,5′-dimethyl-2,2′-dipyridyl | Rep 1. 0 | 10 | 100 |
| (1 minute exposure) | Rep 2. 0 | 10 | |
| 10 mM 6,6′-dimethyl-2,2′-dipyridyl | Rep 1. 0 | 10 | 100 |
| (10 minute exposure) | Rep 2. 0 | 10 | |
| 10 mM 6,6′-dimethyl-2,2′-dipyridyl | Rep 1. 0 | 10 | 100 |
| (1 minute exposure) | Rep 2. 0 | 10 | |
| Control (Ethanol) (10 minute | Rep 1. 5 | 5 | — |
| exposure) | Rep 2. 5 | 5 | |
| Control (Ethanol) (1 minute | Rep 1. 4 | 6 | — |
| exposure) | Rep 2. 5 | 5 | |
| Control (Untreated) | Rep 1. 3 | 7 | — |
| | Rep 2. 6 | 3 | |

The results presented in Table 18 indicate that following a 10 or a 1 minute exposure of B. Bovis louse eggs to a 10 mM solution of either 5,5′-dimethyl-2,2′-dipyridyl or 6,6′-dimethyl-2,2′-dipyridyl that egg hatching in this ectoparasite could be completely inhibited in this assay.

### Example 24

### Effects of Metal Chelating Agents on Egg Hatching in Haemonchus contortus

The gastrointestinal parasite Haemonchus contortus is a major pathogen of sheep throughout the world. The parasite survives through the ability of the adult worms to attach to the abomasal mucosa of the sheep and draw blood. One adult female can take in approximately 0.1 ml blood per day. The adults live can live for many months with the females producing several hundred eggs per day and infected animals shedding upwards of several thousand eggs per gram of faeces per day onto pasture. The eggs hatch after 1-2 days depending on weather conditions and following two moults infective L3 larvae appear on the pasture and are consumed by the host. Once in the host the L3 larvae exsheath in the rumen, migrate to the abomasum and begin to feed by burrowing into the mucosa where they progress through 2 further moults. Infected sheep loose condition and in severe cases suffer dehydration and anaemia due to blood loss. If the parasites are not removed animals will die. Control is centred on the strategic use of anthelmintics coupled with pasture management. Increasing problems with parasite resistance are posing significant problems for producers as the majority of the anthelmintics on the market are no longer effective against this parasite. Indeed, even ivermectin which had shown significant potency for controlling this parasite has begun to fail to the development of resistance.

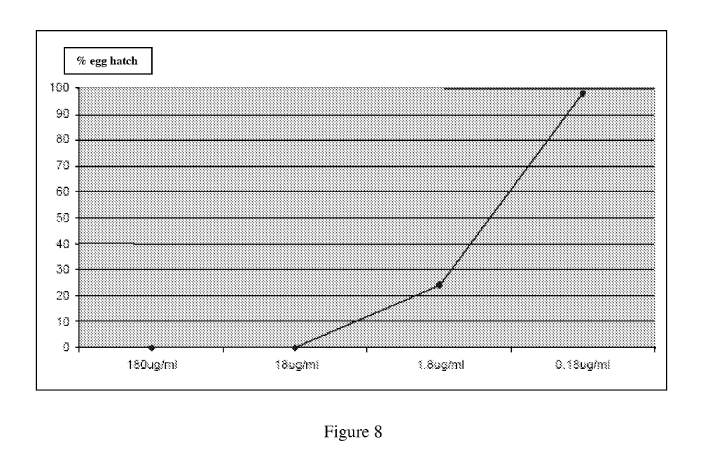

In an attempt to improve control of H. contortus the effects of the compound 5,5′-dimethyl-2,2′-dipyridyl was examined on H. contortus eggs. Eggs were recovered from the faeces of infected sheep using a standard sucrose floatation method. The eggs were corrected to a density of approximately 2,500 eggs per mL and were then exposed to varying concentrations of compound and incubated for 48 hours at 25° C. The eggs were then examined to determine the ovicidal efficacy of the compound in comparison to untreated control and a solvent only control. FIG. 8 shows the ovicidal efficacy of 5,5′-dimethyl-2,2′-dipyridyl on H. contortus eggs.

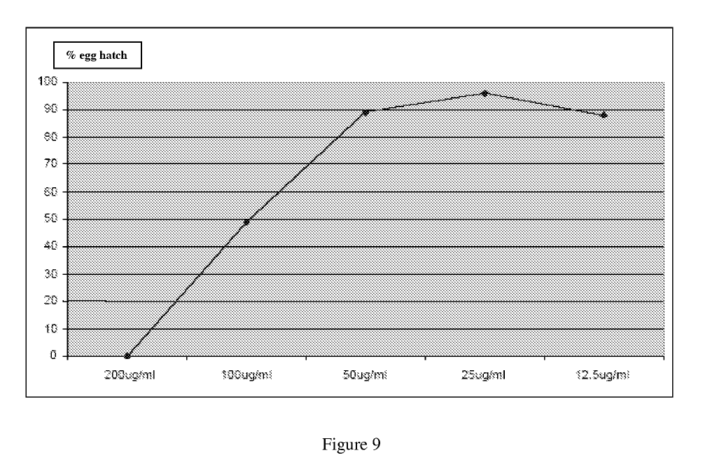

FIG. 8 indicates that 5,5′-dimethyl-2,2′-dipyridyl was potently ovicidal at 180 and 18 ug/mL (equivalent to 1 and 0.1 mM of the active respectively). A comparison of the ovicidal efficacy of 5,5′-dimethyl-2,2′-dipyridyl to the commercial product ivermectin indicated that 5,5′-dimethyl-2,2′-dipyridyl was in the order of 10× more effective at inhibiting H. contortus egg hatching compared to ivermectin (FIG. 8 and FIG. 9).

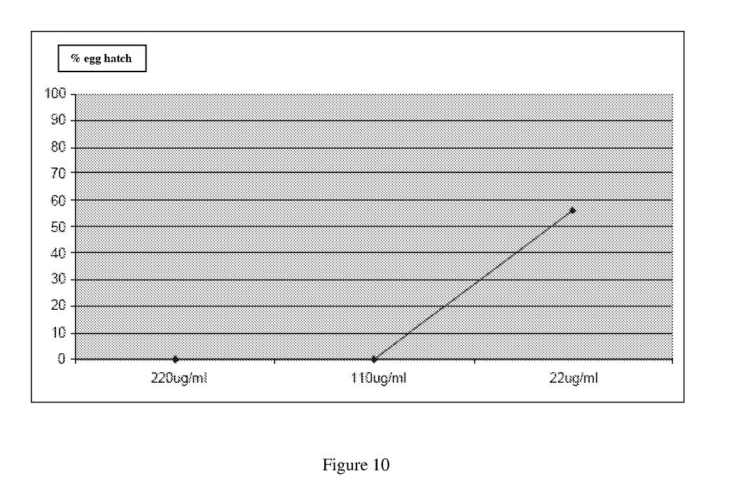

In addition, the ovicidal efficacy of the compound 2-acetyl-1-tetralone was examined against H. contortus (FIG. 10). The same protocol was used as described for 5,5′-dimethyl-2,2′-dipyridyl. The data indicate that at 220 ug/mL (1 mM) and 110 ug/mL (0.5 mM) 2-acetyl-1-tetralone was highly effective at inhibiting egg hatch. Partial inhibition of egg hatching was observed at 22 ug/mL (0.1 mM).

### Example 25

### Effects of 5,5′-dimethyl-2,2′-dipyridyl on Egg Hatching and Viability in House Dust Mite Dermatophagoides spp

A filter paper (90 mm diameter) was taken and placed in a Petri dish of the same dimensions. The filter paper was then wetted throughout with the test compound, using a small air pump sprayer. The wetted filter paper was then allowed to dry in free flowing air. 200 mg dust mite medium, containing roughly 500 mites/g, was placed on the filter paper and the number of mites counted under a microscope. The arena was then left for 2 weeks on an incubator at 25° C. and 75% RH. After 2 weeks the number of mites in the arena was counted for a second time. This experiment was repeated 3 times with the test compound and a further 3 times using water as a control. The results are presented in table 19.

### Results

TABLE 19
Mite counts after 1 hour and two weeks on the test compound treated
filter papers and the control

| | Replicate | 1 hour count | 2 week count |
|---|---|---|---|
| 1 | Test compound | 76 | 74 |
| 2 | Test Compound | 61 | 50 |
| 3 | Test Compound | 104 | 90 |
| 4 | Control | 69 | 110 |
| 5 | Control | 72 | 125 |
| 6 | Control | 91 | 112 |

The mite populations on the treated filter papers showed a small decline over the 2 week period. This may be due to prevention of eggs from hatching and/or to effects on oviposition of the female mites. In contrast, the control treatments showed small increases in mite populations, suggesting a lower mortality of adult mites and no adverse effect on egg viability.

### Example 26

### Effects of 5,5′-dimethyl-2,2′-dipyridyl on Egg Hatching in the Cat Fleas Ctenocephalides felis

Cat flea eggs were exposed for 10 minutes to 10 mM 5,5′-dimethyl-2,2′-dipyridyl and then removed from the solution and placed in an incubator and left to hatch. A group of house dust mite eggs were exposed to the vehicle only and were used as controls. A third group remained untreated. Subsequently the eggs were examined and the percentage of eggs that successfully hatched compared to the controls determined.

### Example 27

### Effects of 5,5′-dimethyl-2,2′-dipyridyl on Egg Hatching in Bed Bugs Cimex lectularius

Bed bug eggs were exposed for 10 minutes to 10 mM 5,5′-dimethyl-2,2′-dipyridyl and then removed from the solution and placed in an incubator and left to hatch. A group of house dust mite eggs were exposed to the vehicle only and were used as controls. A third group remained untreated. Subsequently the eggs were examined and the percentage of eggs that successfully hatched compared to the controls determined.

### Example 28

### Effects of 5,5′-dimethyl-2,2′-dipyridyl on Survival in Haemonchus contortus

Third stage H. contortus larvae were exposed to varying concentrations of 5,5′-dimethyl-2,2′-dipyridyl and the effects on moulting from L3 to L4 examined. The larvae were either exsheathed (their L2 sheath was removed chemically) or unexsheathed (their L2 sheaths were intact). The larvae were exposed to varying concentrations of 5,5′-dimethyl-2,2′-dipyridyl added to their culture media of DMEM and the effects on larval survival monitored over time. Following a 30 minute incubation at 37° C., greater than 90% of the exsheathed larvae exposed to 1 and 0.5 mM of the compound appeared dead. In contrast no adverse effects were observed in the unexsheathed larvae compared to the control larvae up to 3 days post exposure to the compound. By day six post exposure greater than 90% of the larvae appeared to have died in the treatment groups while the control larvae appeared healthy. This larvicidal effect on the unexsheathed larvae appeared to be dose dependent as following a 10 μM exposure of the compound the larvae appeared normal up to 6 days post exposure.

### Example 29

### Effect of Formulated 5,5′-dimethyl-2,2′-dipyridyl on Egg Hatching in Body Lice

5,5′-dimethyl-2,2′-dipyridyl was formulated and evaluated in the standard body louse egg assay. Body louse eggs (15-30 per replicate) of varying ages were exposed for 10 minute to the test solutions or a placebo or left untreated, followed by a 1 minute water wash and blotted dry. The eggs were then incubated at 30° C. over the following 10 days post treatment and the percentage of eggs that successfully hatched was determined (Table 20). The results show a strong dose dependency of ovicidal activity when the compound 5,5′-dimethyl-2,2′-dipyridyl is formulated and applied to body louse eggs.

TABLE 20
A summary of the data is presented below. The data is expressed
as the % of eggs that hatched following treatment.

Age of eggs

| Treatment | 24 hr | 48 hr | 96 hr | 120 hr |
|---|---|---|---|---|
| Placebo formulation | 88 | 93 | 92 | 96 |
| Control (Untreated) | 89 | 84 | 93 | 95 |
| 5,5′-dimethyl-2,2′- | 0 | 0 | 0 | 0 |
| dipyridyl (30 mM) | | | | |
| 5,5′-dimethyl-2,2′- | 4 | 7 | 10 | 12 |
| dipyridyl (10 mM) | | | | |
| 5,5′-dimethyl-2,2′- | 21 | 58 | 64 | 65 |
| dipyridyl (5 mM) | | | | |
| 5,5′-dimethyl-2,2′- | 91 | 80 | 82 | 86 |
| dipyridyl (1 mM) | | | | |

### Example 30

### Effect of Formulated 5,5′-dimethyl-2,2′-dipyridyl on Egg Hatching in Head Lice

Gravid female lice were permitted to lay eggs. The eggs were counted, inspected under a light microscope and all eggs that appeared undamaged were allocated to one of two treatment groups. One group was exposed to 5′-dimethyl-2,2′-dipyridyl in a formulation, while the second group was exposed to the formulation only. The protocol was as follows:

Treatments: 5′-dimethyl-2,2′-dipyridyl (20 mM) n=10
Vehicle control only n=10

Exposure time: 10 minutes

Wash time: 1 minute @˜37° C.

Incubation: All eggs were placed at 31° C. in a humid incubator and monitored for signs of development specifically of the eyes and subsequent hatching, see Table 21.

Results:

TABLE 21

| | | |
|---|---|---|
| Treatment | Ha44 | Placebo |

Day1 (09/03)

| | | |
|---|---|---|
| Stage of | 1 eye development | 0 eye development |
| development | 9 no eye dev. | |

Day 4 (13/03)

| | | |
|---|---|---|
| Stage of | 1 hatched | 4 eye development |
| development | 1 eye development | 6 no eye dev. |
| | 8 no eye dev. | |

Day 5 (14/03)

| | | |
|---|---|---|
| Stage of | 1 eye development | 8 eye development |
| development | 8 no eye dev. | 2 no eye dev. |

Day 6 (15/03)

| | | |
|---|---|---|
| Stage of | 1 eye development | 9 eye development |
| development | 8 no eye dev. | 1 no eye dev. |

Day 7 (16/03)

| | | |
|---|---|---|
| Stage of | 1 eye development | 5 hatched |
| development | 8 no eye dev. | 4 eye development, |
| | | 1 no eye dev |

Day 12 (21/03)

| | | |
|---|---|---|
| Stage of | 1 eye development | 6 hatched |
| development | 8 no eye dev. | 3 eye development, |
| | | 1 no eye dev |

The results indicate that a 20 mM formulation of 5′-dimethyl-2,2′-dipyridyl can significantly suppress egg hatching in head lice. Two of the eggs developed but only one of the two eggs hatched. The other 80% failed to hatch. In the Placebo treated group 60% of the eggs hatched, 30% developed into nymphs but did not hatch and 1 egg did not develop at all. This data indicates that a formulation containing 5′-dimethyl-2,2′-dipyridyl can significantly inhibit head lice eggs from hatching.

### Example 31

### Ovicidal effect of 5,5′-dimethyl-2,2′-dipyridyl and 5,5′ diethyl-2,2′-dipyridyl on Egg Hatching in Plutella in the Glass House

A number of treatments and replicates were set up in the glass house using young cabbage plants that were at the 4-5 leaf stage. Plutella xylostella eggs were laid by gravid females on the leaves such that each plant contained 10 eggs. The plants were sprayed with a commercial ovicide or using the compounds 5,5′-dimethyl-2,2′-dipyridyl and 5,5′ diethyl-2,2′-dipyridyl in a formulation at the rate of 200 L/Ha using a track sprayer. Controls of water only or Placebo only were also included. The ovicidal efficacy was monitored over a number of days an the number of eggs hatching and caterpillar larvae emerging recorded (Table 22).

TABLE 22

| | | # LARVAE EMERGED | | | | | |
|---|---|---|---|---|---|---|---|
| TREATMENT | PLANT # | 0-24 h | 24-48 h | 48-72 h | 72-96 h | 96-120 h | 120-144 h |
| Treatment 1. Lannate (Methomyl) | 1 | 0 | 0 | 0 | 1 | 1 | 2 |
| (2.5 mM of active)* | 2 | 0 | 0 | 1 | 0 | 1 | 2 |
| | 3 | 0 | 0 | 1 | 0 | 1 | 2 |
| | 4 | 0 | 0 | 0 | 0 | 0 | 0 |
| | 5 | 0 | 0 | 1 | 1 | 1 | 1 |
| Treatment 2. HT compound 1 (5,5′diethyl-2,2′-dipyridyl | 6 | 0 | 0 | 0 | 0 | 0 | 0 |
| 10 mM (200 L/Ha) No Wetting Agent | 7 | 0 | 0 | 0 | 0 | 0 | 0 |
| | 8 | 0 | 0 | 0 | 0 | 0 | 0 |
| | 9 | 0 | 0 | 0 | 1 | 1 | 1 |
| | 10 | 0 | 1 | 1 | 0 | 1 | 2 |
| Treatment 3. HT compound 2 (5,5′diethyl-2,2′-dipyridyl) | 11 | 0 | 0 | 0 | 0 | 0 | 0 |
| 10 mM (200 L/Ha)* | 12 | 0 | 0 | 0 | 0 | 0 | 0 |
| | 13 | 0 | 1 | 1 | 2 | 2 | 2 |
| | 14 | 0 | 0 | 3 | 3 | 3 | 3 |
| | 15 | 0 | 0 | 1 | 1 | 1 | 1 |
| Treatment 4. HT compound 2 (5,5′dimethyl-2,2′-dipyridyl) | 16 | 0 | 0 | 0 | 0 | 0 | 1 |
| 10 mM (200 L/Ha)* | 17 | 0 | 0 | 0 | 0 | 0 | 0 |
| | 18 | 0 | 0 | 0 | 0 | 0 | 0 |
| | 19 | 0 | 0 | 0 | 1 | 1 | 1 |
| | 20 | 0 | 0 | 1 | 2 | 2 | 2 |
| Treatment 5. HT compound 2 (5,5′dimethyl-2,2′-dipyridyl) | 21 | 0 | 0 | 0 | 1 | 1 | 1 |
| 20 mM (200 L/Ha)* | 22 | 0 | 0 | 0 | 0 | 0 | 0 |
| | 23 | 0 | 0 | 0 | 0 | 0 | 0 |
| | 24 | 0 | 0 | 0 | 0 | 0 | 0 |
| | 25 | 0 | 0 | 0 | 0 | 0 | 0 |
| Treatment 6. Negative control | 26 | 2 | 5 | 8 | 8 | 8 | 10 |
| (Vehicle only, Placebo)* | 27 | 0 | 3 | 8 | 10 | 10 | 10 |
| | 28 | 0 | 1 | 9 | 9 | 9 | 10 |
| | 29 | 0 | 0 | 2 | 7 | 8 | 10 |
| | 30 | 0 | 5 | 9 | 10 | 10 | 10 |
| Treatment 7. Negative control | 31 | 3 | 0 | 9 | 10 | 10 | 10 |
| (Water) | 32 | 3 | 0 | 10 | 10 | 10 | 10 |
| | 33 | 0 | 5 | 8 | 10 | 10 | 10 |
| | 34 | 0 | 5 | 6 | 10 | 10 | 10 |
| | 35 | 0 | 0 | 10 | 10 | 10 | 10 |

*All these formulations contained wetting agent at 0.3 ml/L

The results from this experiment indicate that both dipyridyl compounds produced significant ovicidal activity on cabbage plants compared to the placebo and untreated groups. In addition, the results were comparable to the product Lannate containing methomyl.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which was included in the present specification is solely for the purpose of providing a context for the present invention. It is not to be taken as an admission that any or all of these matters form part of the prior art base or were common general knowledge in the field relevant to the present invention as it existed in any country before the priority date of each claim of this application.

### REFERENCES

Al-Sayah, M. H., McDonald, R., Branda, N. R., Euro. J. Org. Chem., 2004, 173-182.
Buhleier, E., Wehner, W., Vögthe, F., Chem. Ber., 1978, 111, 200-204.
Busvine, J. R., Entomology and evolution. Antenna. 1993, 17: 196-201.
Busvine, J. R., Biology of the parasites. Cutaneous Infestations and Insect Bites (M. Orkin and H. I. Maibach, eds). 1985, pp. 163-174. New York: Marcel Dekker.
Imperiali, B. and Fisher, S. L., J. Org. Chem., 1992, 57, 757-759.

1. A method of treating pest infestation comprising decreasing exsheathment, excystment, apolysis or inhibiting metamorphosis of an invertebrate by externally contacting a pest with a compound of formula (I): 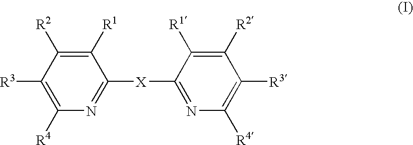
wherein X is selected from a covalent bond, —C(R⁵)₂—, —Z— or —C(R⁵)₂—Z—C(R⁵)₂—;
R¹ and R^{1′} are independently selected from hydrogen, C₁₋₆alkyl, a branched-chain C₁₋₆alkyl, C₂₋₆alkenyl, C₂₋₆alkynyl, hydroxy, C₁₋₆alkyloxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R^{1′} taken together are—
C(R⁵)₂—, —C(R⁵)₂—C(R⁵)₂—, —CR⁵═CR⁵—, C(O), C(S) or NH;
R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or
R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;
each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;
each R⁶ is independently selected from hydrogen and halogen; and
Z is selected from a covalent bond, —NH—, —O—, —S—, —C(O)— and —C(S)—;
a pharmaceutically, veterinary or agriculturally acceptable salt thereof in an amount effective to inhibit or decrease the rate of exsheathment, excystment, apolysis or metamorphosis of said invertebrate.

2-4. (canceled)

5. The method of claim 1 wherein R³ and R^{3′} is each independently ethyl or methyl.

6. The method of claim 5 wherein R³ is ethyl or methyl.

7. (canceled)

8. The method of claim 5 wherein R^{3′} is methyl or ethyl.

9. (canceled)

10. The method of claim 5 wherein R^{3′} is ethyl and R³ is methyl.

11. The method of claim 5 wherein either R^{3′} is methyl and R³ is ethyl.

12. The method of claim 1 wherein both R^{3′} and R³ are ethyl.

13. The method of claim 1, wherein said compound is a metal chelating agent, wherein the metal chelating agent has at least two polar atoms capable of simultaneously coordinating with a metal ion, has a clogP value between 1 and 4; and/or and a molar refractivity in the range of 40 to 90 cm³/mole.

14. The method of claim 13, wherein the metal chelating agent is not 1,10-phenanthroline.

15. The method of claim 13, wherein the metal chelating agent is not 2,2′-bipyridine.

16. The method of claim 1 wherein said method further comprises contacting said pest with a second pesticide.

17-22. (canceled)

23. The method of claim 1, wherein said method produces a greater decrease in the rate of exsheathment, excystment, apolysis or metamorphosis than is seen with the administration of 1,10 phenanthroline.

24. A method of killing an invertebrate pest, said method comprising contacting said pest with a compound of formula (I): 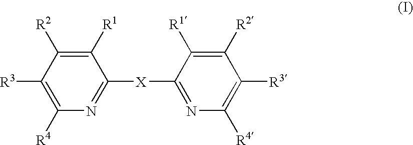
wherein X is selected from a covalent bond, —C(R⁵)₂—, —Z— or —C(R⁵)₂—Z—C(R⁵)₂—;
R¹ and R^{1′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R^{1′} taken together are —C(R⁵)₂—, —C(R⁵)₂—C(R⁵)₂—, —CR⁵═CR⁵—, C(O), C(S) or NH;
R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or
R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;
each R is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;
each R⁶ is independently selected from hydrogen and halogen; and
Z is selected from a covalent bond, —NH—, —O—, —S—, —C(O)— and —C(S)—;
or a pharmaceutically, veterinary or agriculturally acceptable salt thereof in an amount effective to kill said invertebrate.

25. (canceled)

26. The method of claim 24 wherein said invertebrate is selected from the group consisting of nematodes, trematodes, cestodes, lice, fleas, mites and scabies, moths, beetles, caterpillars butterflies, termites, arachnids, cockroaches, centipedes, fleas and mites.

27. A method of inhibiting a remodelling event in an invertebrate population comprising contacting said pest with a compound of formula (I): 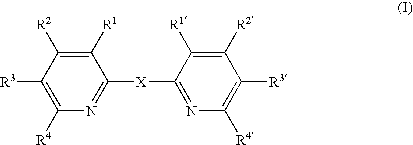
wherein X is selected from a covalent bond, —C(R⁵)₂—, —Z— or —C(R⁵)₂—Z—C(R⁵)₂—;
R¹ and R^{1′} are independently selected from hydrogen, C₁₋₆-alkyl, a branched-chain C₁₋₆alkyl, C₂₋₆alkenyl, C₂₋₆alkynyl, hydroxy, C₁₋₆alkyloxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R^{1′} taken together are —
C(R⁵)₂—, —C(R⁵)₂—C(R⁵)₂—, —CR⁵═CR⁵—, C(O), C(S) or NH;
R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or
R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;
each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;
each R⁶ is independently selected from hydrogen and halogen; and
Z is selected from a covalent bond, —NH—, —O—, —S—, —C(O)— and —C(S)—;
or a pharmaceutically, veterinary or agriculturally acceptable salt thereof in an amount effective to inhibit said invertebrate remodelling event, wherein said invertebrate remodelling event is not egg hatching and said invertebrate is not an ectoparasitic insect.

28. The method of claim 27 wherein R³ and R^{3′} is each independently ethyl or methyl.

29. (canceled)

30. The method of claim 27 wherein said invertebrate pest is selected from the group consisting of nematodes, trematodes and cestodes.

31. A method of inhibiting egg hatching in a non-ectoparasitic invertebrate an invertebrate population comprising contacting said invertebrate with a compound of formula (I): 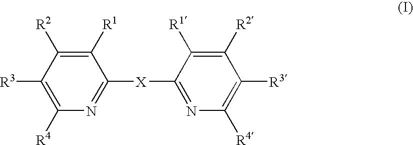
wherein X is selected from a covalent bond, —C(R⁵)₂—, —Z— or —C(R⁵)₂—Z—C(R⁵)₂—;
R¹ and R^{1′} are independently selected from hydrogen, C₁₋₆-alkyl, a branched-chain C₁₋₆alkyl, C₂₋₆alkenyl, C₂₋₆alkynyl, hydroxy, C₁₋₆alkyloxy, thiol, C₁₋₆alkylthio, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R^{1′} taken together are—
C(R⁵)₂—, —C(R⁵)₂—C(R⁵)₂—, —CR⁵═CR⁵—, C(O), C(S) or NH;
R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or
R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;
each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;
each R⁶ is independently selected from hydrogen and halogen; and
Z is selected from a covalent bond, —NH—, —O—, —S—, —C(O)— and —C(S)—;
or a pharmaceutically, veterinary or agriculturally acceptable salt thereof in an amount effective to inhibit said egg hatching.

32. The method of claim 31 wherein R³ and R^{3′} is each independently ethyl or methyl.

33-36. (canceled)

37. A method of controlling or killing an invertebrate pest population comprising internally or externally contacting said pest with a compound of formula (I): 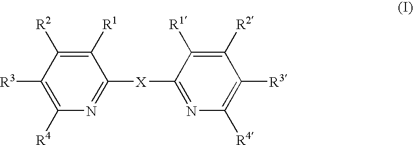
wherein X is selected from a covalent bond, —C(R⁵)₂—, —Z— or —C(R⁵)₂—Z—C(R⁵)₂—;
R¹ and R^{1′} are independently selected from hydrogen, C₁₋₆-alkyl, a branched-chain C₁₋₆alkyl, C₂₋₆alkenyl, C₂₋₆alkynyl, hydroxy, C₁₋₆alkyloxy, thiol, halogen, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, or R¹ and R^{1′} taken together are—
C(R⁵)₂—, —C(R⁵)₂—C(R⁵)₂—, —CR⁵═CR⁵—, C(O), C(S) or NH;
R², R²′, R³, R³′, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or
R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;
each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;
each R⁵ is independently selected from hydrogen and halogen; and
Z is selected from a covalent bond, —NH—, —O—, —S—, —C(O)— and —C(S)—;
or a pharmaceutically, veterinary or agriculturally acceptable salt thereof in an amount effective to kill or reduce the population size of said invertebrate pest.

38. The method of claim 37 wherein R³ and R³′ is each independently ethyl or methyl.

39. (canceled)

40. A method of selecting a chelating agent as a candidate inhibitor of invertebrate remodelling events from a collection of metal chelating agents;
said method comprising selecting a metal chelating agent that has at least two polar atoms capable of simultaneously coordinating with a metal ion and
i. a clogP value between 1 and 4; and/or
ii. a molar refractivity in the range of 40 to 90 cm³/mole.

41. (canceled)

42. A method of selecting a chelating agent as a candidate inhibitor of invertebrate remodelling events from a collection of metal chelating agents of formula I: 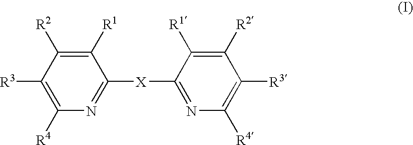
wherein X is selected from a covalent bond, —C(R⁵)₂—, —Z— or —C(R⁵)₂—Z—C(R⁵)₂—;
R¹ and R^{1′} are independently selected from hydrogen, C₁₋₆-alkyl, a branched-chain C₁₋₆alkyl, C₂₋₆alkenyl, C₂₋₆alkynyl, hydroxy, C₁₋₆alkyloxy, thiol, C₁₋₆alkylthio, halogen, C(R⁶)₃, CO₂H, CO₂Cl₁₋₆alkyl, SO₃H, SO₃C₁₋₆alkyl, NH₂, NHC₁₋₆alkyl or N(C₁₋₆alkyl)₂, or R¹ and R^{1′} taken together are—
C(R⁵)₂—, —C(R⁵)₂—C(R⁵)₂—, —CR⁵═CR⁵—, C(O), C(S) or NH;
R², R^{2′}, R³, R^{3′}, R⁴ and R^{4′} are independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, halogen, CN, C(R⁶)₃, CO₂H, CO₂C₁₋₆alkyl, SO₃H, SO₃C₁₋₆alkyl, NH₂, NHC₁₋₆alkyl or N(C₁₋₆alkyl)₂, —CH₂CHNH(CO₂H), NH(C₁₋₆alkylene)N(C₁₋₆alkyl)₂ or a 5 or 6 membered carbocyclic or heterocyclic ring; or
R² and R³ or R³ and R⁴ and/or R^{2′} and R^{3′} or R^{3′} and R^{4′} taken together with the carbon atoms to which they are attached form a 5 or 6 membered carbocyclic or heterocyclic ring;
each R⁵ is independently selected from hydrogen, C₁₋₆alkyl, a branched-chain C₁₋₆alkyl, C₂₋₆alkenyl, C₂₋₆alkynyl, hydroxy, C₁₋₆alkoxy, thiol, C₁₋₆alkylthiol, CO₂H, CO₂C₁₋₆alkyl, SO₃H, SO₃C₁₋₆alkyl, NH₂, NHC₁₋₆alkyl or N(C₁₋₆alkyl)₂;
each R⁶ is independently selected from hydrogen and halogen; and
Z is selected from a covalent bond, —NH—, —O—, —S—, —C(O)— and —C(S)—;
said method comprising selecting a metal chelating agent of formula (I) that has at least two polar atoms capable of simultaneously coordinating with a metal ion and
i) a clogP value of between 1 and 4; and/or
ii) a molar refractivity in the range of 40 to 90 cm³/mole.
